(19) United States
(12) Patent Application Publication
(10) Pub. No.: US 2018/0166174 A1
(43) Pub. Date: Jun. 14, 2018

(54) Disease Mitigation and Elimination Health Learning Engine

(71) Applicant: Thomas J. Lewis, Talbott, TN (US)

(72) Inventor: Thomas J. Lewis, Talbott, TN (US)

(21) Appl. No.: 15/667,297

(22) Filed: Aug. 2, 2017

Related U.S. Application Data
(60) Provisional application No. 62/370,054, filed on Aug. 2, 2016.

(51) Int. Cl. G16H 50/30; G16H 50/20; G16H 10/60; G16H 10/20; A61B 5/021

(52) U.S. Cl. CPC G16H 50/30; G16H 50/20; G16H 10/60; A61B 5/7275; A61B 5/021; G01N 2800/32; G01N 2800/50; G16H 10/20

(57) ABSTRACT

Described is a novel, new, inexpensive approach to screen, perform early diagnosis (on asymptomatic and symptomatic subjects for example), diagnose, establish root causes, and treat subjects. A series of medical steps, each of which is designed to provide the administering healthcare provider with both subjective and objective risk, health and cause evaluation information provides a guide a practitioner to treatments that prevent, slow, delay, stop, or reverse the chronic disease conditions at the root of their cause. Each step in the process provides intelligence about cause and effect. The sum of the steps, when evaluated based on patient outcome, is the basis of a chronic disease health learning engine that leads to continuous improvement of medical knowledge, disease, and methods of healing and treatments to improve patient outcomes.

### CROSS-REFERENCE TO RELATED APPLICATIONS

This application claims the benefit of U.S. Provisional Patent Application No. 62/370,054, filed on Aug. 2, 2016.

### BACKGROUND

Chronic diseases and conditions such as Cancer, cardiovascular (heart) diseases, metabolic disorders, dementias and other neurodegenerative diseases, gastrointestinal diseases, autoimmune diseases, neurological conditions including depression and other mood disorders, inflammatory conditions such as rheumatoid arthritis, musculoskeletal diseases, kidney diseases, oral cavity diseases, and respiratory diseases are among the most common and costly of all health problems. As of 2012, about half of all adults—117 million people in the US alone—had one or more chronic health conditions. One of four adults had two or more chronic health conditions. [Ward B W, Schiller J S, Goodman R A. Multiple chronic conditions among US adults: a 2012 update. Prey Chronic Dis. 2014; 11:130389.] Seven of the top 10 causes of death in 2010 in the US were chronic diseases. Two of these chronic diseases—heart disease and cancer—together accounted for nearly 48% of all deaths. [Centers for Disease Control and Prevention. Death and Mortality. NCHS FastStats Web site. http://www.cdc.gov/nchs/fastats/deaths.htm. Accessed Dec. 20, 2013.] Obesity is a serious health concern. During 2009-2010, more than one-third of adults, or about 78 million people in the US, were obese (defined as body mass index [BMI]≥30 kg/m2). Nearly one of five youths aged 2-19 years was obese (BMI≥95th percentile). [Centers for Disease Control and Prevention. http://www.cdc.gov/nchs/data/factsheets/factsheet_obesity.htm. Accessed Dec. 20, 2013.] Arthritis is the most common cause of disability. Of the 53 million adults with a doctor diagnosis of arthritis, more than 22 million say they have trouble with their usual activities because of arthritis. [Barbour K E, Helmick C G, Theis K A, et al. Prevalence of doctor-diagnosed arthritis and arthritis-attributable activity limitation—United States, 2010-2012. MMWR. 2013; 62(14):869-73. http://www.cdc.gov/mmwr/preview/mmwrhtml/mm6244a1.htm. Accessed Mar. 13, 2014.] Diabetes is the leading cause of kidney failure, lower-limb amputations other than those caused by injury, and new cases of blindness among adults. [Centers for Disease Control and Prevention. National Diabetes Fact Sheet, 2011. Atlanta, Ga.: Centers for Disease Control and Prevention, US Dept. of Health and Human Services; 2011]. Worldwide, nearly 44 million people have Alzheimer's or a related dementia and only 1-in-4 people with Alzheimer's disease have been diagnosed. [Alzheimer.net, http://www.alzheimers.net/resources/alzheimers-statistics/, accessed Feb. 8, 2016.]

A goal of medicine continues to be to maintain good health or as chronic disease medicine has now become in populations. Most modern medical practices, particularly in chronic disease, are directed at managing disease once it has become clinically relevant. One impediment to early chronic disease intervention and prevention is the lack of tests with bona fide disease predictive power and risk stratification capabilities. Further, most tests are “disease specific” rather than broad assessments of human health and risk. Thus doctors are faced with the challenge of choosing and administering multiple tests based on a risk hunch derived from a patient's history and current health status. As a consequence, the main preventative test, that being the standard health physical, has not evolved over the past 100+ years and is well recognized as lacking predictive power for future morbidity and mortality.

Risk score methods exist and the latest attempts at developing scores are improvements over the past systems. The Framingham Risk Score, based on the famous Framingham Heart Study claims to provide a subject's risk of having a heart attack or dying from heart disease within 10 years. However, vast experience subsequent to the availability of this score demonstrates that it is a poor risk tool. In very old people from the general population with no history of cardiovascular disease, concentrations of homocysteine alone can accurately identify those at high risk of cardiovascular mortality, whereas classic risk factors included in the Framingham risk score do not. [De Ruijter, Wouter, et al. “Use of Framingham risk score and new biomarkers to predict cardiovascular mortality in older people: population based observational cohort study.” Bmj 338 (2009).] Currently recommended risk scoring methods derived from the Framingham study may significantly overestimate the absolute coronary risk assigned to individuals in the United Kingdom. [Brindle, Peter, et al. “Predictive accuracy of the Framingham coronary risk score in British men: prospective cohort study.” Bmj 327.7426 (2003): 1267.]

The Reynolds Risk Score is designed to predict your risk of having a future heart attack, stroke, or other major heart disease in the next 10 years. It is similar to Framingham but adds hsCRP and family information. Introduction of hsCRP into cardiovascular risk assessments can refine the risk status of symptom-free subjects, especially among intermediate risk middle-age women. [Móczár, Csaba. “Comparison of SCORE and Reynolds cardiovascular risk assessments in a cohort without cardiovascular disease.” Orvosi hetilap 154.43 (2013): 1709-1712.] In primary prevention, Reynolds Risk Score underestimates the number of subjects at risk of future CHD events. [Desai, Milind Y., et al. “Reclassification of cardiovascular risk with coronary calcium scoring in subjects without documented coronary heart disease: Comparison with risk assessment based on Reynolds Risk Score.” Journal of the American College of Cardiology 59.13s1 (2012): E1186-E1186.]

The intermountain risk score uses complete blood count and basic metabolic profile to predict mortality. Intermountain Risk Score, a predictor of mortality, was associated with morbidity endpoints that often lead to mortality. [Horne, Benjamin D., et al. “The Intermountain Risk Score (including the red cell distribution width) predicts heart failure and other morbidity endpoints.” European journal of heart failure 12.11 (2010): 1203-1213.]

Evaluating these three risk scores demonstrate that: adding measures, particularly those associated with inflammation, including hsCRP and white blood cell counts, improves the predictive capability of the risk score. Current methods of identifying and quantifying chronic disease risk rely on indirect assumption, an inadequate breadth of test parameters, and lack evaluation of actual tissue. The vast majority of chronic disease cases are only diagnosed after the disease has expressed clinically relevant or life-effecting change on a person. The predominant existing model for chronic disease diagnosis and management involves a set of tests, after a subject falls ill, that are presumably targeting a specific chronic disease or symptom. Continued proliferation of these diseases illustrates the failing of this approach. As a result, there is currently no clear methodology on how to predict and stratify risk of current or future disease morbidity and mortality. The risk and health evaluation tools within this chronic disease health learning engine, referred to as the Living Profile™ and Chronic Disease Temperature™ of this invention, fills this significant unmet need, especially when coupled to advanced testing including stealth ectopic infection and treatment thereof and when the testing and treatment is performed in a iterative loop of continuous health improvement.

### BRIEF SUMMARY OF THE INVENTION

Example embodiments of the present general inventive concept can be achieved by providing a method for determining the chronic or specific disease risk level of a patient, comprising: interviewing a patient and acquiring the patient's blood or related testing,having the patient complete a questionnaire related to the patient's health and assigning risk values to the questionnaire answers; applying the answers from the questionnaire and the patient's blood or related testing to determine risk value scores for at least one category of health risk, using the risk value scores to determine which set of at least one biomarker tests to perform and performing the at least one biomarker tests on the patient to generate at least one biomarker test results, determining a raw value for each of the at least one biomarker test results, comparing the raw value for the at least one biomarker test to known threshold values related to the biomarker to determine at least one chronic disease temperature increment for each of the at least one biomarker tests; calculating an overall chronic disease temperature value by summing a base chronic disease temperature score with the at least one chronic disease temperature increments; implementing a disease mitigation treatment plan for the patient based on the results provided from the overall chronic disease temperature value, and iteratively repeating the steps above until the overall chronic disease temperature value falls within a predetermined acceptable threshold.

Example embodiments of the present general inventive concept can be achieved by providing a computer software application for determining the chronic or specific disease risk level of a patient, comprising: an interface which is configured to provide a questionnaire related to the patient's health, lifestyle and risk for disease and to gather the answers to the questionnaire, an analyzer that classifies the patient into risk categories and degrees of risk based on the answers to the questionnaire to generate an overall risk score for each category of disease and that matches the risk scores with a set of at least one biomarker tests, a processor which receives as input raw data related to the set of at least one biomarker test and generates a set of chronic disease temperature increments as output, and then applies the chronic disease temperature increments to a base chronic disease temperature score to generate an overall chronic disease temperature score, memory for saving the answers to the questionnaire, the overall risk scores, the results of the biomarker tests, the chronic disease temperature increments and the overall chronic disease temperature score, wherein, the computer application is programmed to repeat the steps above after the patient has implemented a disease mitigation program provided by a physician until the overall chronic disease temperature score is under a predetermined threshold value. By “physician” in this context it is meant a physician, health coach, healthcare provider, or self-directed by the patient.

### BRIEF DESCRIPTION OF THE DRAWINGS

The above-mentioned features of the invention will become more clearly understood from the following detailed description of the invention read together with the drawings in which:

FIG. 1 shows a method for assessing the health state and chronic disease state of a subject.

FIG. 2 shows an actual example of the Living Profile™ survey questions, answers, and logic.

FIG. 3 shows the RealHealth patient/participant dashboard displaying several health parameters including the Living Health Profile Risk Score.

FIG. 4A shows the results of clicking on the Living Health Profile Risk Score.

FIG. 4B shows the connection between specific biomarkers and chronic disease categories and conditions.

FIG. 4C shows disease specific biomarkers contributing to disease-specific temperatures.

FIG. 5 shows the homocysteine contribution to CDT.

FIG. 6 shows significant changes in F2-isoprostanes.

FIG. 7 shows the results of meta-analysis conducted on the relationship between CRP and mortality.

FIG. 8 shows the total maximum contribution to the CDT from WBC.

FIG. 9 shows the total maximum contribution to the CDT from Vitamin D levels.

FIG. 10A shows Lp-PLA2 Activity—Morbidity, and Mortality.

FIG. 10B shows Lp-PLA2 Activity—Morbidity, and Mortality.

FIG. 10C shows Lp-PLA2 Activity—Morbidity, and Mortality.

FIG. 10D shows Lp-PLA2 Activity—Morbidity, and Mortality.

FIG. 10E shows Lp-PLA2 Activity—Morbidity, and Mortality.

FIG. 10F shows Lp-PLA2 Activity—Morbidity, and Mortality.

FIG. 10G shows Lp-PLA2 Activity—Morbidity, and Mortality.

FIG. 10H shows Lp-PLA2 Activity—Morbidity, and Mortality.

FIG. 11 shows the mortality risk in heart failure with TNFα.

FIG. 12 shows best mortality predictive value and accuracy for sTNF-R1.

FIG. 13 shows mean serum levels of TNF according to stages of diabetic retinopathy.

FIG. 14 shows the step-up in risk of disease with F2-isoprostanes level in serum.

FIG. 15A shows the concentration of F2-isoprostanes with respect to Alzheimer's disease and the degree of Cortical Atrophy.

FIG. 15B shows the concentration of F2-isoprostanes with respect to Alzheimer's disease and the degree of Cortical Atrophy.

FIG. 16 shows F2-isoprostane total maximum contribution to the CDT calculation.

FIG. 17 shows Red Blood Cell Distribution Width and Mortality.

FIG. 18 shows Red Blood Cell distribution width total maximum contribution to the CDT calculation.

FIG. 19 shows HbA1c total maximum contribution to the CDT calculation.

FIG. 20 shows the leptin/adiponectin ratio total maximum contribution to the CDT calculation.

FIG. 21 shows the age-adjusted mortality rates per 1000 person-years by fibrinogen quintiles.

FIG. 22 shows 16-years all-cause mortality rates in middle-aged men in relation to plasma levels of inflammation-sensitive proteins.

FIG. 23 shows the fibrinogen total maximum contribution to the CDT calculation.

FIG. 24 shows Uric Acid CDT increments.

FIG. 25 shows CHF incident rate.

FIG. 26 shows ESR total maximum contribution to the CDT calculation.

FIG. 27 shows the Kaplan-Meier mortality curves by TNF-alpha quartile.

FIG. 28 illustrates survival compared to TNF-alpha surrogate quartiles.

FIG. 29 shows the mean serum IL-8 and TNF-alpha levels according to the stages of diabetic retinopathy.

FIG. 30 shows TNF-alpha total maximum contribution to the CDT calculation.

FIG. 31A shows the Kaplan-Meier estimates for major adverse cardiovascular events.

FIG. 31B shows the Kaplan-Meier estimates for death, myocardial infarction, and stroke according to quartiles of beta 2 microglobulin.

FIG. 32 shows the beta-2-microglobulin total maximum contribution to the CDT calculation.

FIG. 33A shows an example of the association of myeloperoxidase with total and cardiovascular mortality in individuals undergoing coronary angiography.

FIG. 33B shows an example of the association of myeloperoxidase with total and cardiovascular mortality in individuals undergoing coronary angiography.

FIG. 33C shows an example of the association of myeloperoxidase with total and cardiovascular mortality in individuals undergoing coronary angiography.

FIG. 33D shows an example of the association of myeloperoxidase with total and cardiovascular mortality in individuals undergoing coronary angiography.

FIG. 34 shows the myeloperoxidase total maximum contribution to the CDT calculation.

FIG. 35A shows theT-proBNP Level by Quartile of Test Score.

FIG. 35B shows the percent of Participants with Poor Performance by NT-proBNP Quartile.

FIG. 36 shows the higher concentrations of NT-proBNP at baseline associated with greater subsequent mortality.

FIG. 37 shows the NT-proBNP total maximum contribution to the CDT calculation.

FIG. 38 shows the Hazard Ratios and Diseases associated with elevated Cystatin C.

FIG. 39 shows the cystatin C total maximum contribution to the CDT calculation.

FIG. 40 shows the chlamydia pneumoniae total maximum contribution to the CDT calculation.

FIG. 41 shows the white blood cell subtype and cardiovascular hazard ratio.

FIG. 42 shows the NLR total maximum contribution to the CDT calculation.

FIG. 43 shows the normal levels for neutrophil counts in presumed healthy subjects.

FIG. 44 shows a J-shaped association between neutrophil counts and mortality.

FIG. 45 shows the neutrophil counts total maximum contribution to the CDT calculation.

FIG. 46A shows an example of the Age-Related Eye Disease Study Illustrating the Probability of Death Associated with Eye Diseases.

FIG. 46B shows an example of the Age-Related Eye Disease Study Illustrating the Probability of Death Associated with Eye Diseases.

FIG. 46C shows an example of the Age-Related Eye Disease Study Illustrating the Probability of Death Associated with Eye Diseases.

FIG. 46D shows an example of the Age-Related Eye Disease Study Illustrating the Probability of Death Associated with Eye Diseases.

FIG. 46E shows an example of the Age-Related Eye Disease Study Illustrating the Probability of Death Associated with Eye Diseases.

FIG. 46F shows an example of the Age-Related Eye Disease Study Illustrating the Probability of Death Associated with Eye Diseases.

FIG. 47 shows a flowchart summarizing the role of Inflammation in the Pathogenesis of Glaucoma.

FIG. 48 shows a high level representation of one embodiment of the invention.

FIG. 49A shows an example of the Health Learning Engine Description and Predictive Use.

FIG. 49B shows an example of the Health Learning Engine Description and Predictive Use.

FIG. 49C shows an example of the Health Learning Engine Description and Predictive Use.

FIG. 49D shows an example of the Health Learning Engine Description and Predictive Use.

FIG. 49E shows an example of the Health Learning Engine Description and Predictive Use.

FIG. 49F shows an example of the Health Learning Engine Description and Predictive Use.

FIG. 50A shows an example of the Health Learning Engine Description and Predictive Use.

FIG. 50B shows an example of the Health Learning Engine Description and Predictive Use.

FIG. 50C shows an example of the Health Learning Engine Description and Predictive Use.

FIG. 50D shows an example of the Health Learning Engine Description and Predictive Use.

FIG. 50E shows an example of the Health Learning Engine Description and Predictive Use.

FIG. 50F shows an example of the Health Learning Engine Description and Predictive Use.

FIG. 51A shows an example of the Health Learning Engine Description and Predictive Use.

FIG. 51B shows an example of the Health Learning Engine Description and Predictive Use.

FIG. 51C shows an example of the Health Learning Engine Description and Predictive Use.

FIG. 52A shows an example of the Health Learning Engine Description and Predictive Use.

FIG. 52B shows an example of the Health Learning Engine Description and Predictive Use.

FIG. 52C shows an example of the Health Learning Engine Description and Predictive Use.

FIG. 52D shows an example of the Health Learning Engine Description and Predictive Use.

FIG. 53A shows an example of the Health Learning Engine Description and Predictive Use.

FIG. 53B shows an example of the Health Learning Engine Description and Predictive Use.

FIG. 53C shows an example of the Health Learning Engine Description and Predictive Use.

FIG. 53D shows an example of the Health Learning Engine Description and Predictive Use.

FIG. 54A shows an example of the Health Learning Engine Description and Predictive Use.

FIG. 54B shows an example of the Health Learning Engine Description and Predictive Use.

FIG. 54C shows an example of the Health Learning Engine Description and Predictive Use.

FIG. 54D shows an example of the Health Learning Engine Description and Predictive Use.

FIG. 55A shows an example of the Health Learning Engine Description and Predictive Use.

FIG. 55B shows an example of the Health Learning Engine Description and Predictive Use.

FIG. 56A shows an example of the Health Learning Engine Description and Predictive Use.

FIG. 56B shows an example of the Health Learning Engine Description and Predictive Use.

FIG. 57A shows an example of the Health Learning Engine Description and Predictive Use.

FIG. 57B shows an example of the Health Learning Engine Description and Predictive Use.

FIG. 57C shows an example of the Health Learning Engine Description and Predictive Use.

FIG. 58 shows the association between age-related Macular Degeneration and 15-year Mortality, Cardiovascular Disease and ischemic heart disease Mortality.

FIG. 59 shows the ranking of Specific Disease Temperature Biomarkers.

### DETAILED DESCRIPTION

The present invention describes a novel approach to screen, perform early diagnosis (on asymptomatic and symptomatic subjects for example), diagnose, establish root causes, and treat subjects to improve outcomes. This invention includes a series of steps, each of which is designed to provide the administering healthcare provider with both subjective and objective risk, health, and cause evaluation information, and provides a guide to the practitioner for treatments that prevent, slow, delay, stop, or reverse the chronic disease conditions at the root of the cause. Importantly, each step in the process provides intelligence about cause and effect. The sum of the steps, when evaluated based on patient outcome, is the basis of a chronic disease health learning engine that leads to continuous improvement, and provides medical knowledge regarding disease and methods of healing and treatments, in order to improve patient outcomes.

This engine “learns” by altering the risk values assigned to the subjective information in response to the calculated chronic disease temperature, which internalizes the risk factors associated with the objective information. The health learning engine also may alter the risk values assigned to the objective information in response to the statistical analysis of morbidity and/or mortality data associated with the specific measurements constituting the objective information. These alterations may be performed once, or iteratively. The subjective information is obtained from patient health information and accepted historical health risks associated with the patient health information. The objective information is obtained from blood or related testing, where blood or related testing includes measured pathology, blood testing, physiology, and blood or related testing. Thus, the system can “learn” how the blood or related testing parameters accurately measure patient health and correlate these data back to the patient health information.

A chronic disease mitigation and elimination system and learning engine provides a software interface to a patient for inputting a variety of information regarding their health, condition, behaviors, environment, attitudes, diagnoses, drugs, blood or related testing, and the like. The results of this data may be fed into a software application analyzer which makes determinations regarding which set of biomarker tests should be provided to the patient based on risk levels for particular diseases and conditions. For example, where the data reveals a heightened risk of cardiovascular disease, biomarkers which provide diagnostic information regarding cardiovascular disease may be ordered. The software application may be optimized to identify the minimum number of biomarkers to provide the maximum amount of information regarding the patient's risks for chronic and/or specific diseases. The results of the biomarker tests may be compared to known or experimental threshold values for the biomarkers and then fed into a processor to calculate a set of chronic disease temperature increments. The processor may be computer hardware or could be implemented in software. These chronic disease temperature increments may then be summed with a base chronic disease temperature score in order to generate an overall chronic disease temperature score. Computer memory may be used to store the acquired and calculated data described above and in the description below. The overall score may be applicable to one disease, or the patient's overall health. The above steps may be iteratively repeated until the overall chronic disease temperature score falls below a desirable threshold.

This chronic disease mitigation and elimination system and learning engine facilitates the determination whether a subject has risk or decaying health that make the subject susceptible for current/immediate and future chronic disease and also expresses the magnitude of the current or future risk in subjects with or without current diagnosable disease. The process includes detailed subject lifestyle evaluation and testing, testing and measuring for biomarkers, each of which provide information about general chronic disease risk and for specific chronic conditions, further diagnostics based on results of preliminary testing, root-cause analysis, treatments, and health creation solutions. Further diagnosis to determine disease causes and treatment is an important output from this system. Repetition of the steps in the chronic disease mitigation and elimination system provides those skilled in the art a roadmap of diagnostic discovery and an objective way to measure efficacy of treatments selected in the first round of testing and an opportunity to adjust treatments to achieve a general chronic disease or specific chronic disease temperature of 98.6 (which infers essentially no current or future risk and active disease) or as close to that value as practical.

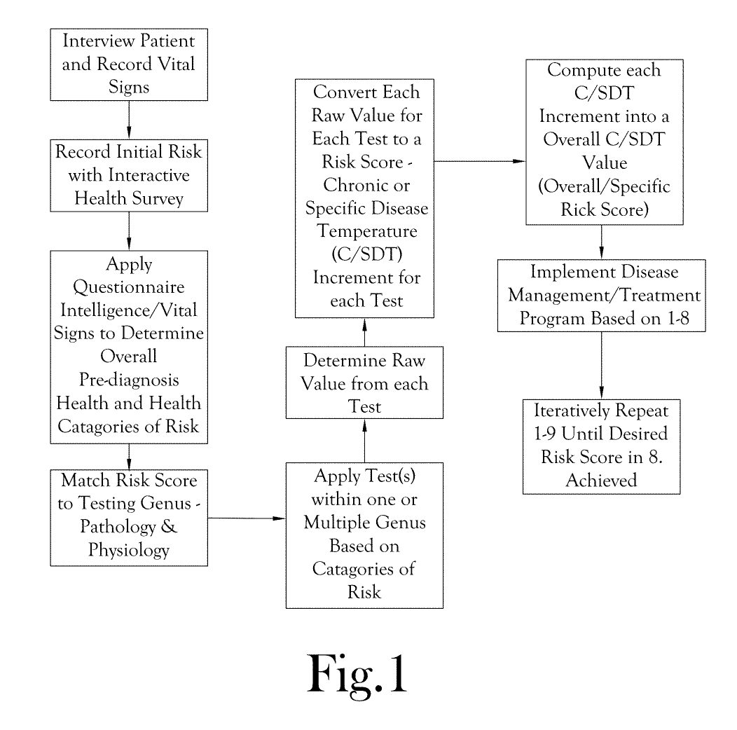

Box 1 in FIG. 1 references a method for assessing the health state and chronic disease state of a subject. The health professional evaluates the physical state of the subject through observation. In addition, blood or related testing are obtained and recorded including but not limited to heart rate, heart rate variability, pulse regularity, blood pressure, body mass index, age, short-term memory, grip strength, health complaints, perceived stress levels, perceived energy levels, nutrition, sleep patterns, unusual lumps, bumps, moles, cold sores, and rashes, reflex, breathing patterns, and core body temperature. Each value is assigned a numeric risk score based on an algorithm that includes age, sex, and the measured value, Table 1

TABLE 1
Box 1 of FIG. 1. Interview Patients and Record Vital Signs

| | | Risk | |
|---|---|---|---|
| Measurement | Values | Ranges | Type |
| Age | | 0-# | D |
| BMI | <18.5->=30 | 0-# | D, M |
| Resting Heart/pulse rate | 30-150 | 0-# | D, H |
| Noted arrhythmias | Normal, abnormal, suspect AFIB | 0-# | D, H |
| Heart rate variability | Optimal, suboptimal, unhealthy | 0-# | D, H |
| Blood pressure | Normal, low, high, very high | 0-# | D, H |
| Short term memory | Abbreviated MMSE | 0-# | D, N |
| Grip strength | Mean (lbs) −75%. −50%, −25%, −10% | 0-# | D |
| Stress level (perceived) | High, medium, low, none | 0-# | D |
| Energy level (perceived) | High, medium, low, none | 0-3# | D |
| Nutritional intake | Optimal - poor | 0-# | D, M |
| Sleep patterns (perceived) | >8 h, <8 h, interrupted | 0-# | D |
| Dermatological evaluation | Clean, moles, sores, rashes | 0-# | D, C |
| Core body temperature | High, low, normal | 0-# | D, M |
| Reflex | Normal, low | 0-# | D, N |

D = chronic disease general;
H = cardiovascular disease;
M = metabolic disease;
N = neurological/neurodegenerative disease;
C = Cancer.
These values are presented as examples and are not intended to be comprehensive.

In a follow-on method for assessing the health and chronic disease state of a subject, the subject completes a survey of questions related to their health, condition, behaviors, environment, attitudes, diagnoses, drugs and other questions pertaining to their past, current, and future health. The survey selectable answers to each question are each assigned a risk value for general and specific chronic disease states and overall health. A mathematical algorithm powering the survey calculates the relative risks, with respect to chronic diseases for the survey taker, based on their answers. An example of the types of questions and answers are provided in Table 2. The number of health related questions includable in the survey has no limit. The intent of the specific invention is: 1. To be efficient in asking most health-impactful questions, 2. Limit the length of the survey to approximately 30 minutes, and 3. Have the ability to add as many questions as deemed necessary to improve upon the final risk score from the survey. This last part, number 3 is a key component of the health learning engine.

TABLE 2
Box 2 of FIG. 1. Risk Survey Criteria (examples)

| | | | Risk |
|---|---|---|---|
| Measurement/Question | Values/Answers | Risk Assigned | Categories |
| Age | Ranges | Single value | D |
| Sex | Male/Female | Single value | |
| Occupation history | Varied | Single value | D, S |
| Home states | Varied | Single value | D |
| Travel History | Varied | Single value | D |
| Physical activity | Not, modestly, very | Single value | D, M, N, C, S |
| Favorite activities | Varied | Single value | D, M, N, C, S |
| Pets | Yes, No, Farm | Single value | D, N, S, H |
| | Animals | | |
| Pets | Indoor, outdoor | Single value | D, N, S, H |
| Sun Exposure | Varied | Single value | D, C, H, N, S, M |
| What's for Dinner | Varied | Single value | D, C, H, N, S, M |
| Frequently consumed food | Varied | Single value | D, C, H, N, S, M |
| types | | | |
| Most frequented restaurants | Varied | Single value | D, C, H, N, S, M |
| Favorite beverages | Varied | Single value | D, C, H, N, S, M |
| Salt usage | Salt, sea salt, | Single value | D, H |
| | frequency | | |
| Sugar usage | Type, frequency | Single value | D, M |
| Cooking oils | Varied | Single value | D, C, H, N, S, M |
| Breakfast | Varied | Single value | D, C, H, N, S, M |
| Allergies | Varied | Summation | D, G, A |
| Allergens | Varied | Summation | D, G, A |
| Supplements | Varied | Summation | D, C, S, H, G |
| Nicotine Status | Varied | Single value | D, C, H |
| Recreational substances | Varied | Single value | D, N |
| Past diagnoses | Varied | Summation | D, C, H, N, S, M |
| Health Today | Varied | Summation | D |
| Colds/Flu | Frequency | Single value | D, C, H, N, S, M |
| Surgeries/procedures | Varied | Summation | D, S, N |
| Brain | Varied | Single value | D, N, M |
| Short-term memory | Good, bad | Single value | D, N, M |
| Heart | Varied | Summation | D, C, H, N, M |
| GI Tract | Varied | Summation | D, N, G, H |
| Oral health | Varied | Summation | D, C, H, N, S, M |
| Oral hygiene | Varied | Summation | D, C, H, N, S, M |
| Eye | Varied | Summation | D, C, H, N, S, M |
| Musculoskeletal | Varied | Summation | D, C, S, M |
| Respiratory | Varied | Summation | D, R |
| Urinary Tract | Varied | Summation | D |
| Skin | Varied | Summation | D, C |
| Sleep | Varied | Summation | D, N |
| Toxicity | Varied | Summation | D, C |
| Stress | None, Normal, High/ | Single value | D |
| | Frequency | | |
| Women's Issues | Varied | Summation | D, N |
| Pathogens | Varied | Summation | D, C, H, N, S, M |
| Medication categories | Varied | Summation | D, C, H, N, S, M |
| Drugs | Varied | Summation | D, C, H, N, S, M |
| Supplements | Varied | Summation | D, C, H, N, S, M |

D = chronic disease general;
H = cardiovascular disease;
M = metabolic disease;
N = neurological/neurodegenerative disease;
C = Cancer,
S = musculoskeletal,
A = Autoimmune,
G = Gastrointestinal.
A limitless set of risk categories are assignable to a question or question/answer combination.

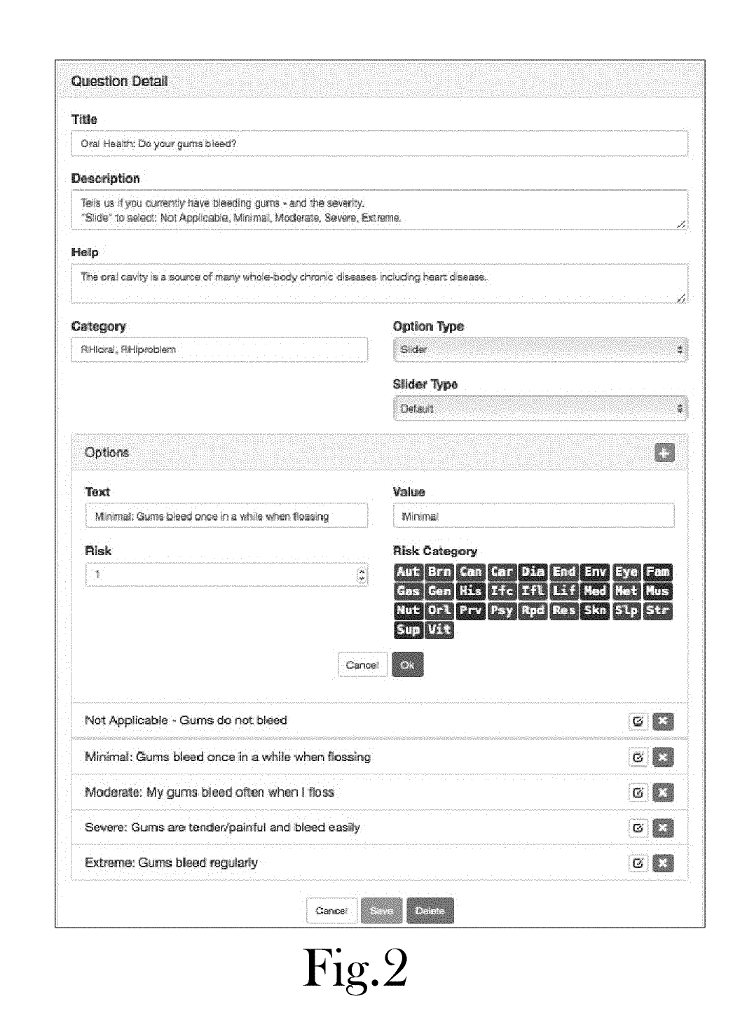

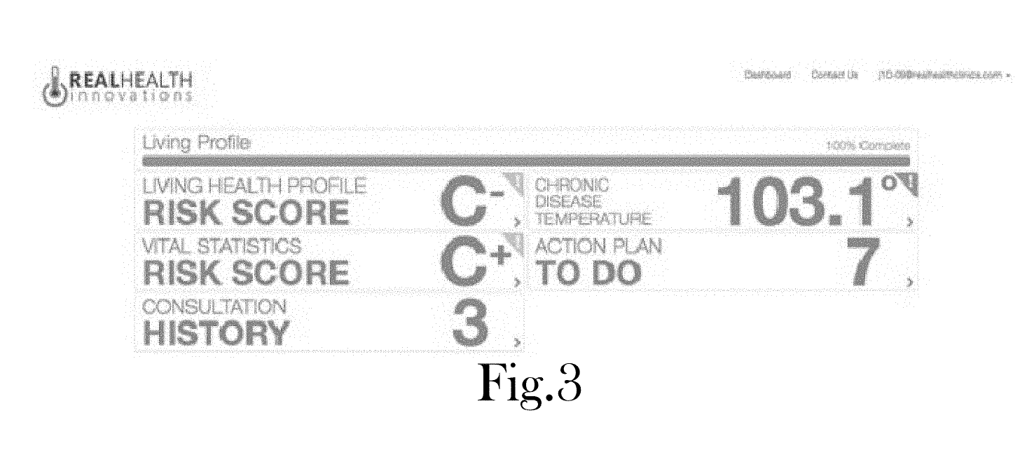

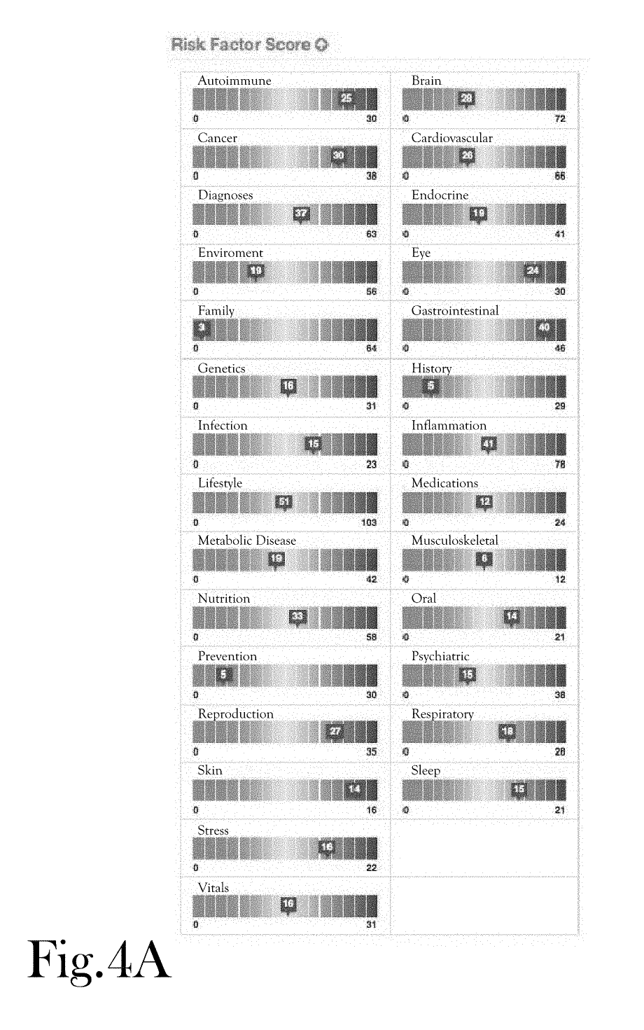

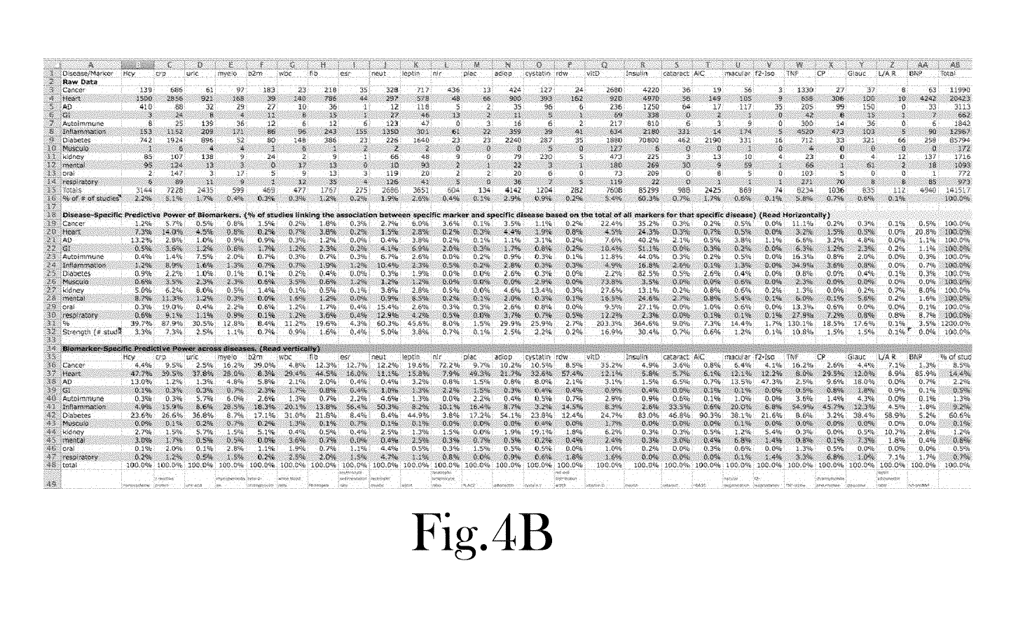

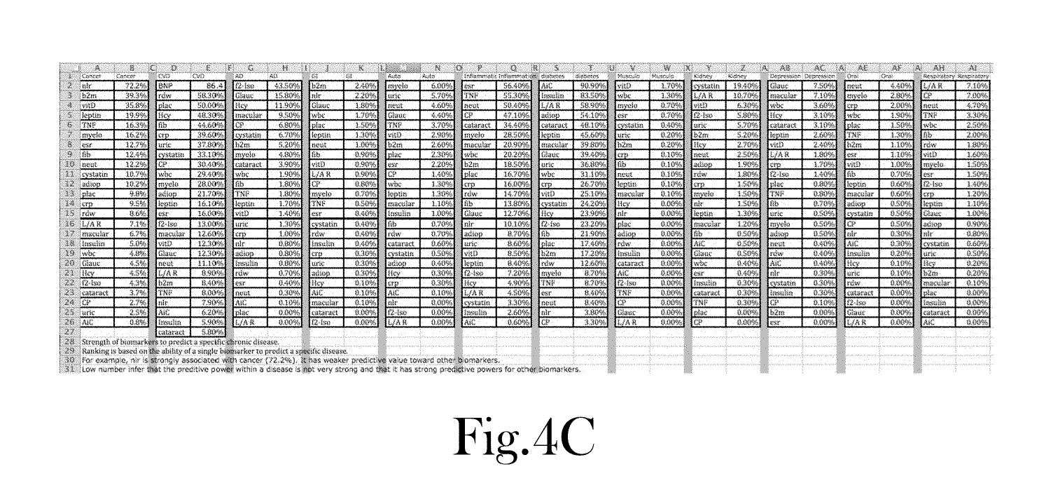

FIG. 2 shows an actual example of the Living Profile™ survey questions, answers, and logic. FIG. 3 shows the RealHealth patient/participant dashboard displaying several health parameters including the Living Health Profile Risk Score. The Risk Score of C− is derived from the aggregate of all risk values assigned to answers in the Living Profile assessment. The sum of the risk values is assigned to a letter grade, in this case C−, based on a range of values assigned to each incremental letter grade. FIG. 4A shows the results of clicking on the Living Health Profile Risk Score. The subcategories of risk are revealed—called the Risk Factor Score. The numeric marker indicates the relative risk for the patient/participant in multiple categories of health, risk, and disease. In this example, 29 categories of risk are represented in the report. The position and the size of the numeric value indicate the magnitude of the risk in each category with “0” being no presumed risk and an arbitrary upper value being the presumed maximum risk. This upper risk and risk range is adjustable with an increase in data input into the health learning engine of this invention. For example, the interface can include a display configured to display a health dashboard including the Living Profile™ and the Chronic Disease Temperature™ or other disease-specific health temperatures and other pertinent health information. A software system that gathers health outcome data, measures, analyzes, and compares data so as to rate protocols as to their ability to create, improve, and optimize health.

In a follow-on method for assessing the health and chronic disease state of a subject, the subject undergoes tests for physiological, pathophysiological, and pathological biomarkers. These tests may be performed independently of previous methods, or the results from previous methods may be used to determine which biomarker tests to perform and improve the efficiency of testing. Health risk values, assigned as “temperature increments,” are pre-assigned to laboratory values and/or ranges of values for the blood biomarker and ocular tests based upon rigorous evaluation of biomarker/tissue pathology and consequential morbidity or mortality. The major endpoint in determining temperature increments for each biomarker is mortality. Specifically, a temperature increment is first assigned to a biomarker at a laboratory value for that biomarker where the first statistically increased increase in human mortality is noted. For a given biomarker, increases in assigned temperature increments correspond with increasing raw laboratory values for biomarker in association with further increases in mortality. Statistical analysis on the increase in mortality risk is evaluated for each biomarker with consideration to risk ratios, statistical “P” values and published tertiles, quartiles, quintiles, deciles, and other available scale representations of mortality risk. These temperature increments are summed for each test used in the biomarker evaluation with the result being the subject's overall “temperature” or risk above a normal level with preference toward chronic disease in general or a specific chronic disease as a function of the predictive power of the biomarker. FIG. 4B shows the connection between specific biomarkers and chronic disease categories and conditions. This total temperature is added to 98.6 to give the subject their CDT and their “specific” disease temperature where the specific disease is one of the following but not limited to Cancer, cardiovascular (heart) diseases, dementias and other neurodegenerative diseases, gastrointestinal diseases, autoimmune diseases, neurological conditions including depression and other mood disorders, inflammatory conditions such as rheumatoid arthritis, musculoskeletal diseases, kidney diseases, oral cavity diseases, and respiratory diseases. FIG. 4C shows disease specific biomarkers contributing to disease-specific temperatures.

Subjects with elevated CDT or chronic disease-specific temperature require intervention to lower or eliminate the excess “temperature” above a baseline of 98.6. The results from the overall testing and specific testing guides a practitioner to follow-on tests to determine both symptoms and causes of the chronic disease condition or conditions. This coupled to ameliorating risks identified in the Living Profile constitutes a comprehensive treatment and health creation plan. Any or all of the methods addressed here are repeated to access the efficacy of therapy. The process is repeated until the subject's CDT or disease specific temperature is 98.6 or as close as deemed achievable by the health care professional skilled in the art.

In one aspect, the invention includes a series of biological tests to be obtained for specific biomarkers, the results of which are used to determine an amount of CDT, in degrees Fahrenheit or degrees Celsius, that each test contributes to the calculation of a human's overall CDT. The CDT is a scale of risk for current or future chronic disease morbidity or mortality, with a focus on a statistical increase in mortality as an endpoint when available. The CDT scale is based on the easily recognizable and understandable core body temperature scale. Core body temperature refers to the temperature of the internal environment of the body. This includes organs such as the heart and liver, as well as the blood. Elevation or depression of the core body temperature is often indicative of acute current and active disease. The temperature 98.6° Fahrenheit (F.) is considered the benchmark for a subject without acute current active disease. The temperature scale increases, to reflect severity of disease, log linearly from 98.6 F to temperatures as high as 111.2 F. In some instances, higher temperatures have been recorded. In general, a subject's core body temperature increases to a statistical maximum of 107.6 F. Temperatures above 107.6 F are almost always associated with quick and sudden death or debilitation. For the purposes of statistical relevance, the CDT scale ranges from 98.6 F, reflecting no immediate or near future risk of chronic disease morbidity or mortality, to 107.6 F reflecting a subject with chronic disease, high near future chronic disease risk, high sudden death risk, or high near future likelihood of death due to chronic conditions. This same scale is applied to specific chronic disease conditions. For example, a subject's CANCER TEMPERATURE™ extends from a no/low risk value of 98.6 to a high risk value of 107.6.

In exemplary embodiments, the biological tests are for, but not limited to, the following biomarkers, each of which confer a unique contribution to a human's chronic disease temperature, dictated by the result of the test and the known risks associated with these results. Biomarker: Homocysteine, C-Reactive Protein, White Blood Cell Count, Vitamin D, Lp-PLA2, Insulin, F2-Isoprostanes, HbA1C, Adiponectin, Leptin, Fibrinogen, Uric acid, Erythrocyte Sedimentation Rate, TNF-alpha, Beta-2-microglobulin, Red Blood Cell Distribution Width, NT-proBNP, Cystatin C, Chlamydaphilia Pneumoniae, Myloperoxidase, eGFR, UACR, UAER, total neutrophils, absolute neutrophils, lyme disease, q-fever, and various other obligate intracellular infections based on IGM and IGG values and other measures of the prevalence of infection.

In one aspect, the invention includes a series of tissue tests involving the evaluation and measurement of tissue or disease pathologies in the eye, the results of which are used to determine an amount of CDT in degrees Fahrenheit or Celsius that each test contributes to a the calculation of a human's overall CDT The tests include, but is not limited to, evaluation of: Macular Degeneration, Cataract, and Glaucoma.

In one aspect, the invention includes the summation of risk values from each test and the addition of the risk values summation to 98.6 to arrive at the estimated CDT and specific disease temperature of the human.

In one embodiment, the chronic disease associated with or contributing to the chronic disease burden of a human may be cardiovascular disease and the many diseases associated with that label including, but not limited to: atherosclerosis, stroke, coronary artery disease, high blood pressure, cardiac arrest, congestive hear failure, arrhythmia, peripheral artery disease, congenital heart disease.

In one embodiment, the chronic disease associated with or contributing to the chronic disease burden of a human may be a metabolic disease including, but not limited to: diabetes (type 1, 2, or 3), metabolic syndrome X, metabolic brain diseases, lipid metabolism disorders, mitochondrial diseases.

In one embodiment, the chronic disease associated with or contributing to the chronic disease burden of a human may be a neurodegenerative disease including, but not limited to: dementia, Alzheimer's disease, Parkinson's disease, ALS, glaucoma, Huntington's disease, multiple sclerosis, and mild cognitive impairment.

In one embodiment, the chronic disease associated with or contributing to the chronic disease burden of a human may be an autoimmune disease including, but not limited to: rheumatoid arthritis, type 1 diabetes, multiple sclerosis, vasculitis, alopecia areata, lupus, polymyalgia rheumatic, ankylosing spondylitis, celiac disease, Syogren's syndrome, and temporal arteritis.

In one embodiment, the chronic disease associated with or contributing to the chronic disease burden of a human may be any form of cancer.

In one embodiment, the chronic disease associated with or contributing to the chronic disease burden of a human may be gastrointestinal diseases including, but not limited to ulcers, acid reflux, celiac disease, irritable bowel syndrome, inflammatory bowel diseases, diverticulitis, cirrhosis, colitis, constipation, diarrhea, dyspepsia, incontinence, gallstone, hepatitis, lactose intolerance, Whipple's disease.

In one embodiment, the chronic disease associated with or contributing to the chronic disease burden of a human may be mood diseases/disorders including, but not limited to: Depression, bipolar disorder, autism, violent and antisocial behavior, addiction, mania, dysthymic disorder, affective disorder, drug dependency.

In one embodiment, the chronic disease associated with or contributing to the chronic disease burden of a human may be musculoskeletal diseases including, but not limited to: arthritis, osteoporosis, osteomalacia, carpal tunnel syndrome, tendonitis, bursitis, muscular dystrophy, myasthenia gravis, and lupus erythematosus.

In one embodiment, the chronic disease associated with or contributing to the chronic disease burden of a human may be respiratory diseases including, but not limited to: asbestosis, asthma, bronchitis, chronic obstructive pulmonary disease, croup, cystic fibrosis, hantavirus, idiopathic pulmonary fibrosis, influenza, lung cancer, pandemic flue, pertussis, pleurisy, pneumonia, pulmonary embolism, respiratory syncytial virus, sarcoidosis, sleep apnea, spirometry, and tuberculosis.

In one embodiment, the chronic disease associated with or contributing to the chronic disease burden of a human may be oral diseases including, but not limited to: gum disease, gingivitis, dental caries, oral cancer, mucosal infection, oral candidiasis, oral infection, and tooth loss.

In one embodiment, the chronic disease associated with or contributing to the chronic disease burden of a human may be kidney diseases including, but not limited to: chronic kidney disease, kidney stones, glomerulonephritis, polycystic kidney disease, and urinary tract infections.

In one embodiment, the chronic disease associated with or contributing to the chronic disease burden of a human may be a disease characterized by chronic inflammation not already included in other classifications and including, but not limited to: allergy, anemia, asthma, autism, Crohn's disease, eczema, fibrosis, Guillain-Barre syndrome, mediated disease, pancreatitis, psoriasis, scleroderma, depression, antisocial behaviors and any other disease the name of which ends in “itis.”

In one embodiment, the chronic disease associated with or contributing to the chronic disease burden of a human may be caused or exacerbated by stealth or detectable pathogens.

In one aspect, the invention includes additional tests for humans with an elevated (above 98.6) CDT. These tests are for causes and exacerbators/accelerators of the chronic disease.

In one aspect, the invention includes treatments to lower the CDT and improve the health of the afflicted human.

The present invention provides biomarkers, and levels of biomarkers useful for the detection, qualification, or quantification of future risk of morbidity or mortality. Each biomarker is a relevant marker for risk for a single or multiple chronic diseases and increased or sudden mortality. The relative and absolute level of the biomarkers contributes to a determination of risk. Taken together, these biomarkers provide much more accurate information compared to a single biomarker. Biomarkers include substances often present is a subject's peripheral blood, urine, saliva, stool, nervous system and lymphatic fluids. Biomarkers also include tissue pathology changes that are readily observed through non-invasive methods. These tissue pathologies are not present in healthy subjects and change, in a graded way, with the progression of a given disease state or condition.

Blood borne biomarkers are, including, but not limited to, homocysteine, c-reactive protein, uric acid, myeloperoxidase, beta-w-microglobulin, total white blood cell count, fibrinogen, erythrocyte sedimentation rate, neutrophil count, neutrophil-to-leukocyte ratio, neutrophil-to-lymphocyte ratio, leptin, adiponectin, leptin-to-adiponectin ratio, lp-lpa2, e-GFR, UACR, UAER, microalbuminuria, cystatin C, red blood cell distribution width, 25-hydroxy vitamin D, 1,25-dihydroxyvitamin D, insulin, HgA1C, f2-isoprostanes, TNF-alpha, chlamydophila pneumoniae, other spirochetes, other intracellular infectious species, molds, fungi, species consider benign in certain tissue but pathogenic in others, prions, archaea, obligate species, omega-6 to omega-3 ratio, total cholesterol, N-Terminal pro Brain Natriuretic Peptide, autoantibodies, IgG, IgA, IgM, lipid profiles, triglycerides, Ceruloplasmin, Albumin, Rheumatoid factor (RF), Anti-cyclic citrullinated peptide antibody (CCP), Anti-nuclear antibody (ANA), Complement, NfKBeta, Cryoglobulins, IL-1, IL-6, OxLDL, ADMA/SDMA, Apolipoprotein A-1, Apolipoprotein B, Lipoprotein (a), NMR LipoProfile, sd-LDL, C-Peptide, Fructosamine, TMAO (Trimethylamine N-oxide), Galectin-3, Coenzyme Q10, PSA, Creatine Kinase, toxoplasmosis, other parasites, worms, h-pylori, infectious species associated with lyme disease, nanobacteria and other infectious species. Tissue pathology markers are, including, but not limited to, nuclear cataract, cortical cataract, subcapsular cataract, glaucoma, macular degeneration, dry eye, amyloidoses, nerve fiber layer volume and thickness, that allow for determining the CDT and disease specific temperature in a subject. Multiple morbidity or mortality markers provide more information compared to a single marker. Current or future risk is best provided by obtaining data for the presence and amount of each biomarker in a subject. For the purposes of the CDT calculation, multiple biomarker tests are required to obtain a meaningful value. Optimally, the sum of the largest assigned temperature increment for each biomarker should equal or exceed “9.” A single biomarker value or any number of biomarkers, the sum value of their maximum temperature increment values being less than “9” may provide an underestimate of the CDT or specific disease temperature of a human. When the sum maximum temperature increments assign to the biomarkers tested exceeds “9,” then the final chronic or specific disease temperature is determined by multiplying the final value by the ratio of 9/sum of the maximum temperature increments for the biomarkers included in the evaluation. The chosen biomarkers may include eye or other pathology measures as tissue changes are more predictive of future risk compared to blood biomarkers. Practitioners choosing to exclude eye and tissue data may do so but at the risk that the chronic disease temperature may be reduced in its predictive value.

The biomarker and tissue panel provided herein allows for identification and characterization of systemic chronic disease burden and resultant morbidity and mortality risk. Through the biomarker and tissue panels and methods of their use as provided herein, a practitioner is able to identify, qualify, and quantify subjects at risk for chronic disease adverse events that may be imminent or likely to occur in the future, the time of which is not specifiable. However, most clinical studies on mortality risk are based on 6, 9, or 15 year risk statistics. The extent of the elevation of the CDT or disease specific temperature signifies increasing risk of the chronic disease adverse event both imminently and in the future. Application of the assays and tests provided herein will help to identify patients with increased risk, there degree of risk, and infer potential causes and methods for ameliorations of the condition(s). Subjects with elevated CDT and disease specific temperature can be placed under high scrutiny through assessment visits and testing and be persuaded to follow advice to lower their CDT and disease specific temperature and improve their health and health outlook. Practitioners may use trend analysis on the level of the CDT and disease specific temperature to determine efficacy of treatments, appropriateness of doses, and other relevant therapeutic conditions to lower the subjects CDT as much as is practicable, with a goal of achieving a lasting CDT and disease specific temperature of 98.6. Thus, measurement of the presence and quantity of the biomarkers and tissue changes provided herein allows for selection and monitoring of efficient risk-reducing treatment to avoid complications associated with an elevated CDT and specific disease temperature, mainly from chronic diseases. Processes and methods that lower the chronic and specific disease temperature constitute the health learning engine.

A large number of biomarkers are known for a variety of chronic conditions. See US/2008/0057590, incorporated by reference in its entirety. However, the present invention is particularly directed to the use of a minimum number of biomarkers to provide a maximum amount of information concerning general and specific chronic disease risk, morbidity, and mortality in a subject. In addition, the strata of risk has not been previously defined for many of these biomarkers particularly with respect to future morbidity and mortality. Importantly, the chosen tests are readily available and of low cost, each of which is offered at most major clinical laboratories. Single blood biomarkers tests alone do not account risk adequately. Many of the physiological tests incorporated into the chronic disease risk calculator are acute phase reactants and their values do not always signify future risk of morbidity or mortality. Repeated testing, over periods of days, weeks, or months enable the practitioner to distinguish transient values from chronic values. The use of multiple biomarkers and tissue lessen the potential for false positives considerably. However, neither the measurement value, nor the prospective trend in value is completely adequate to elucidate the retrospective value for the marker. Tissue changes do, however, reflect both present and past adverse physiological conditions as tissue deteriorates in the presence of continued insult. Just as the HbA1c value is more representative of excess glucose burden over time compared to a simple fasting glucose test, so to is the condition of tissue reflective of chronic disease compared to the one-time or even multiple-time measurement of disease associated biomarkers. Thus the chronic/specific disease temperature™ risk measure is much more predictive of disease risk when it includes tissue change values and physiological biomarkers compared to risk calculators that do not include tissue changes. This is a novel concept in risk stratification and evaluation.

A large number of diseases that reflect deleterious changes in tissue are known. However most of these pathologies are identified only after a chronic disease is diagnosed and thus are not useful for initial chronic disease risk assessment and prevention. The eye provides a modality to assess changes in tissue in both asymptomatic, early-stage symptomatic subjects, while addressing the severity of disease in disease-burdened subjects. Further, the eye, and the techniques and methods for diagnosis, provide for measurement of the health and changes to the health of nervous tissue, vascular tissue, and stem cells at very low cost and non-invasively. The extent of development of an eye disease, similar to the level of a biomarker, is reflective of the extent of either a current or latent chronic disease and risk of further morbidity and potential early mortality. Classification of cataract, macular degeneration, glaucoma, and dry eye is well known. Also, unanticipated high morbidity and mortality from chronic diseases are associated with eye diseases. Several major health studies detail the association between eye diseases and systemic chronic disease morbidity and mortality. A partial listing of these studies is provided in the Table 5.

TABLE 5
Eye studies that demonstrate the relationship between eye pathology and early
mortality.

| Study Name | Representative Reference |
|---|---|
| Age-related eye disease | AREDS Research Group. “Associations of mortality with ocular |
| study (AREDS) | disorders and an intervention of high-dose antioxidants and zinc |
| | in the Age-Related Eye Disease Study: AREDS Report No. 13.” |
| | Archives of ophthalmology 122.5 (2004): 716. |
| Blue Mountain Study | Lee, Anne J., et al. “Open-angle glaucoma and cardiovascular |
| | mortality: the Blue Mountains Eye Study.” Ophthalmology |
| | 113.7 (2006): 1069-1076. |
| Barbados Study | Hennis, Anselm, et al. “Lens opacities and mortality: The |
| | Barbados Eye Studies11The authors have no proprietary interest |
| | in the products or devices mentioned herein.” Ophthalmology |
| | 108.3 (2001): 498-504. |
| Rotterdam Eye Study | Borger, Petra H., et al. “Is there a direct association between age- |
| | related eye diseases and mortality?: The Rotterdam Study.” |
| | Ophthalmology 110.7 (2003): 1292-1296. |
| Beijing Study | Xu, Liang, et al. “Mortality and ocular diseases: the Beijing Eye |
| | Study.” Ophthalmology 116.4 (2009): 732-738. |
| Beaver Dam Study | Klein, Ronald, Barbara EK Klein, and Scot E. Moss. “Age- |
| | related eye disease and survival: the Beaver Dam Eye Study.” |
| | Archives of ophthalmology 113.3 (1995): 333-339. |
| Priverno Eye Study | Nucci, Carlo, et al. “Association between lens opacities and |
| | mortality in the Priverno Eye Study.” Graefe's Archive for |
| | Clinical and Experimental Ophthalmology 242.4 (2004): 289-294. |
| Salisbury Eye Evaluation | West, Sheila K., et al. “Mixed lens opacities and subsequent |
| Project | mortality.” Archives of ophthalmology 118.3 (2000): 393-397. |
| The European Eye Study | Augood, Cristina A., et al. “Prevalence of age-related |
| (EUREYE) | maculopathy in older Europeans: the European Eye Study |
| | (EUREYE).” Archives of ophthalmology 124.4 (2006): 529-535. |
| The Andhra Pradesh Eye | Khanna, Rohit C., et al. “Cataract, visual impairment and long- |
| Disease Study | term mortality in a rural cohort in India: the Andhra Pradesh Eye |
| | Disease Study.” PLoS One 8.10 (2013): e78002. |

Thus, the invention provides biological markers, including blood-based and other biological biomarkers and eye tissue and other tissue/pathology changes that in combinations can be used in a method to measure a subject's risk of chronic disease, risk of future morbidity and mortality, and to determine appropriate therapies, and monitor subjects that are undergoing therapies for chronic disease. Elevated CDT and SPECIFIC DISEASE TEMPERATURE™ allows a caregiver to select or modify therapies or interventions for preventing chronic diseases or helping those already afflicted along with a means to measure the success of interventions, the basis for the HEALTH LEARNING ENGINE™.

Biological and Tissue Biomarkers

A detailed description of blood-based biomarkers for adipose tissue activity, there detection, and their utility in risk assessment are described elsewhere. See WO 2010/076655 A1, incorporated by reference in its entirety. The present invention is particularly directed to the use of a minimum number of biomarkers and tissue markers to provide a maximum amount of information concerning chronic disease risk and future morbidity and mortality in a subject. The invention provides for the detection and quantification of levels of biomarkers in fluids, solids, gases and tissue biomarkers including those in the eye cataract, macular degeneration, glaucoma, and dry eye which in combination with the biological biomarkers are useful markers for risk in both asymptomatic and disease burdened subjects as each allows the assessment of different, complementary, and sometimes overlapping aspects of underlying chronic disease and morbidity and mortality risk.

Studies including more than one assay have proven to have greater value in determining chronic disease risk. As an example, the negative association between higher homocysteine and immediate recall was strongest in persons with a high level of IL-6. [Van den Kommer, T. N., et al. “Homocysteine and inflammation: predictors of cognitive decline in older persons.” Neurobiology of aging 31.10 (2010): 1700-1709.] It has been found that assays involving the measurement of homocysteine, C-reactive protein, and white blood cells in various combinations have greater value in determining chronic disease risk and response to medication than any of these biomarkers alone. Combination of these biomarkers allow attainment of clinically useful sensitivity and specificity. Accordingly, measurements of a biomarker panel comprising or consisting of multiple biomarkers and tissue changes may be used to improve the sensitivity and specificity of a diagnostic test compared to a test involving any one of these biomarkers or tissue changes alone.

Tissue markers are normally used to establish the presence of a specific disease associated with the change in that specific tissue. However, tissue changes often appear to relate, either through association, or causation, or both, to diseases of other tissue not as easily observed or measured. Eye tissue is easily observed, qualified, and quantified due to the transparency of the layers of the eye and the 60 diopter magnification afforded by the lens. Changes to tissue markers in the eye are beginning to be appreciated as associated with tissue changes in other bodily systems and, as described in Table 5, higher morbidity and mortality incidences. Adverse tissue changes in the eye are associated, possibly at a root-cause, thus at a therapeutic level, with adverse changes in tissue outside of the eye. As tissue changes within the eye are often observable, qualifiable, and quantifiable before those in other bodily systems, measurement of eye tissue, and the changes thereof provide for a powerful predictor of latent systemic disease, disease risk, and mortality. We have made the unexpected discovery that assessments involving the measurement of eye tissue markers (pathologies) have great value in measuring disease risk, disease, and the response of the body based on drug and other therapeutic interventions. Combinations of these tissue marker pathologies allow attainment of clinically useful sensitivity and specificity toward risk, risk amelioration, and treatment in diseases beyond the eye, but understood by us to have common mechanisms of development and propagation.

Homocysteine

In various embodiments, homocysteine is used as a biomarker. Homocysteine is a four carbon amino acid containing sulfur in the form of a sulfhydryl group. Homocysteine was discovered in 1932 by the eminent American chemist Vincent DuVigneaud by heating the amino acid methionine in concentrated sulfuric acid. In contrast to methionine, homocysteine does not occur in the peptide linkages of proteins, even though the molecule differs from methionine, an important sulfur amino acid of proteins, only by a methyl group. The importance of the methyl group and its relation to the biochemistry of sulfur were explored in animals by DuVigneaud and many other investigators in the 1930s and 1940s. However, the importance of homocysteine in human disease was totally unknown until 1962, when cases of the disease homocystinuria were discovered in children with arterial and venous thrombosis, mental retardation, and other disturbances of the central nervous system. Analysis of vascular disease occurring in cases of homocystinuria caused by different inherited enzymatic abnormalities of methionine metabolism, revealed the atherogenic effect of homocysteine in causing arteriosclerotic arterial plaques. This concept is termed the homocysteine theory of arteriosclerosis, since many important aspects of atherogenesis occurring in the general population are attributed to the effect of homocysteine on the cells and tissues of the arteries.

Homocysteine values are useful as a predictive biomarker for homocystinuria, vitamin B12 deficiency, and folate deficiency. Homocysteine has clinical use for the assessment of risk of cardiovascular disease, stroke and dementia (including Alzheimer's disease). Early diagnosis and homocysteine-lowering therapy are important to minimize the effects of certain metabolic disorders. Furthermore, homocysteine may be an independent predictor of stroke and dementia, including Alzheimer disease. [Seshadri S, Beiser A, Selhub J, et al. Plasma homocysteine as a risk factor for dementia and Alzheimer's disease. N Engl J Med. 2002; 346:476-483.] In the Framingham study, involving primarily people of European descent, a 5 μmol/L increase in plasma homocysteine level was associated with a 40% increase in the 8-year risk of Alzheimer disease. [Seshadri S, Beiser A, Selhub J, et al. Plasma homocysteine as a risk factor for dementia and Alzheimer's disease. N Engl J Med. 2002; 346:476-483.] Dr. Kilmer McCully, the pioneer of the homocysteine theory of cardiovascular disease estimates that elevated blood homocysteine accounts for at least 10% of the risk of coronary heart disease in the U.S. population.

Homocysteine levels are now shown to track, in a dose dependent manner, with the severity of chronic disease. Diseases of the central nervous system are found in patients with severe hyperhomocysteinemia. Epidemiological studies show a positive, dose-dependent relationship between mild-to-moderate increases in plasma total homocysteine concentrations and the risk of neurodegenerative diseases, such as Alzheimer's disease, vascular dementia, cognitive impairment, or stroke. [Herrmann, Wolfgang, and Rima Obeid. “Homocysteine: a biomarker in neurodegenerative diseases.” Clinical Chemistry and Laboratory Medicine 49.3 (2011): 435-441.] Increased concentrations of pro-inflammatory blood cytokines and plasma homocysteine are frequently reported in Alzheimer's disease (AD). Homocysteine appears to have immunomodulating and pro-inflammatory activities. Further, emerging evidence from animal and non-AD human studies implicates homocysteine in potentiating the activities of proinflammatory cytokines; homocysteine toxicity may also, in part, be mediated by these cytokines. [Veryard, Leon, et al. “Pro-inflammatory cytokines IL-1β and TNF-α are not associated with plasma homocysteine concentration in Alzheimer's disease.” Current Alzheimer Research 10.2 (2013): 174-179.]

Homocysteine concentrations predict the risk of mortality in patients with known coronary artery disease; mortality ratios across quartiles of homocysteine concentrations are 1.0 (<9.0 μmol/L), 1.9 (9.0-14.9 μmol/L), 2.8 (15.0-19.9 μmol/L), and 4.5 (≥20 μmol/L). [Nygard O, Nordrehaug J E, Refsum H, et al. Plasma homocysteine levels and mortality in patients with coronary artery disease. N Engl J Med. 1997; 337:230-236.] Among American participants in the Third National Health and Nutrition Examination Survey (NHANES III), higher plasma homocysteine concentrations were associated with increasing cardiovascular mortality risk (HR 1.30, 1.02-1.66, p=0.032). [Wu C K, Chang M H, Lin J W, Caffrey J L, Lin Y S. Renal-related biomarkers and long-term mortality in the US subjects with different coronary risks. Atherosclerosis. 2011; 216:226-36. doi: 10.1016/j. atherosclerosis.2011.01.046 PMID: 21371709]. Significant increases in cardiovascular mortality were demonstrated in those patients in the highest quartile of plasma homocysteine were the quartiles were assigned as follows: (in micromoles per liter): Q1, 4.13-9.25; Q2, 9.26-11.43; Q3, 11.44-14.25; and Q4, 14.26-219.84. The mortality analysis is presented in Table 6 below.

TABLE 6
Homocysteine and Mortality

| | RR (95% CI) | |
|---|---|---|
| | Total Mortality | CVD Mortality |
| Variable | (n = 653 Events) | (n = 244 Events) |
| tHcy ≥14.26 μmol/L | | |
| Unadjusted | 2.18 (1.86-2.55) | 2.17 (1.66-2.82) |
| Adjusted | 1.54 (1.31-1.82) | 1.52 (1.16-1.98) |
| Age (per year increase) | 1.11 (1.09-1.12) | 1.10 (1.06-1.12) |
| Sex (female) | 0.62 (0.52-0.73) | 0.52 (0.39-0.69) |
| Diabetes | 1.77 (1.43-2.19) | 2.36 (1.74-3.25) |
| Smoking | 1.59 (1.31-1.93) | 1.49 (1.07-2.07) |
| Systolic blood pressure | 1.11 (1.02-1.20) | 1.29 (1.15-1.46) |
| (per 20-mmHg increase) | | |
| Total cholesterol | 0.97 (0.93-1.00) | 1.10 (1.00-1.14) |
| (per 0.52-mmol/L increase) | | |
| HDL cholesterol | 0.98 (0.95-1.01) | 0.95 (0.91-1.00) |
| (per 0.13 mmol/L increase) | | |

* Relative risk (RR) estimates and 95% confidence intervals (Cis) for total and cardiovascular disease (CVD) mortality, comparing the uppermost to lower three quartiles of nonfasting plasma total homocysteine (tHcy), and other potential independent predictor variables. Relative risk estimates were adjusted for all variables listed in the table, with the exception of the unadjusted tHcy analyses. HDL indicates high-density lipoprotein.

From: Bostom, Andrew G., et al. “Nonfasting plasma total homocysteine levels and all-cause and cardiovascular disease mortality in elderly Framingham men and women.” Archives of internal medicine 159.10 (1999): 1077-1080.

Homocysteine elevation is more commonly associated with the following conditions: homocystinuria (cystathionine-β-synthase deficiency); vitamin B12 (MMA increased) and folate deficiency (MMA not increased); cardiovascular disease; chronic renal disease (typically 9-50 μmol/L); increasing age; male sex; MTHFR mutations; hypothyroidism; selected malignancies (eg, breast, ovarian, and pancreatic cancer); diets rich in methionine (high meat intake); cigarette smoking; and treatment with corticosteroids, methotrexate, nitrous oxide, cyclosporine, vitamin B6 antagonists (isoniazid, azauridine, penicillamine, procarbazine), and anticonvulsants (phenytoin, carbamazepine), and premature mortality.

Homocysteine reference ranges vary. An example of a reference values by age are as follows: [Ferri F F, ed. Laboratory Tests and Interpretation of Results. Ferri's Clinical Advisor, 1st ed. Elsevier Mosby; 2012.; Section IV:]

Age 0-30 years: 4.6-8.1 μmol/L

Age 30-59 years: 6.3-11.2 μmol/L (males); 4-5-7.9 μmol/L (females)

Age>59 years: 5.8-11.9 μmol/L

The reference range of urine homocysteine (24-hour urine collection) varies with the technique used, from 0-9 μmol/g creatinine.

In exemplary embodiments, homocysteine values <6.3 μmol/L may be considered the upper limit for good health in all people under the age of 50. This is based on an increased risk of atherosclerosis, heart attack and stroke. [Broxmeyer L. Heart disease: the greatest ‘risk’ factor of them all. Med Hypotheses. 2004; 62:773-779.] After age 50, a target upper limit value for homocysteine is <7.8 μmol/L due to a number of age-related confounding factors that may lead to homocysteine level increases. However, epidemiological studies have shown that higher homocysteine levels are associated with higher risk, even at levels that are considered “normal.” [Robinson K, Mayer E L, et al. Hyperhomocysteinemia and low pyridoxal phosphate: common and independent reversible risk factors for coronary artery disease. Circulation. 1995; 92:2825-2830.]

A limited set of compounds have been shown to affect homocysteine concentrations in a subject. Vitamin B12 and folate do lower homocysteine levels. Studies to determine whether lowering homocysteine levels can reduce the risk of heart disease haven't shown a benefit. Reducing foods high in animal protein also is reported to lower homocysteine. A growing body of research on marine lipids, rich in omega-3 polyunsaturated fatty acids (PUFAs), reveals that omega-3 rich fish oil supplementation can reduce elevated homocysteine levels. Homocysteine levels in the treatment group declined as much as 3.10 μmol/L; glycolsylated hemoglobin (HbA1C, a measure of long-term sugar levels in the blood) decreased in the treatment group and increased in the control group. [Pooya Sh, Jalali M D, et al. The efficacy of omega-3 fatty acid supplementation on plasma homocysteine and malondialdehyde levels of type 2 diabetic patients. Nutr Metab Cardiovasc Dis. 2010; 20:326-331.]

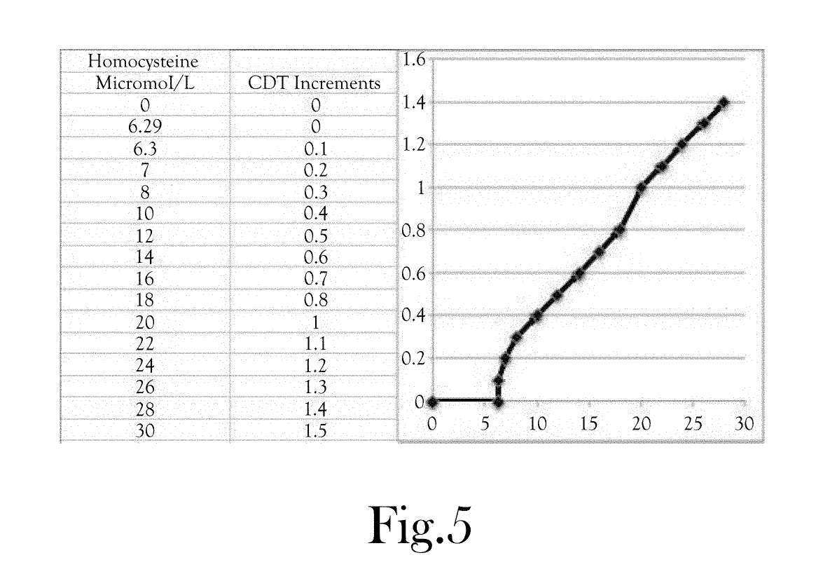

In various embodiments, homocysteine contributes to a subject's chronic disease temperature as follows: Total maximum contribution to the CDT calculation is 1.5° F. (0.83° C.). See FIG. 5.

C-Reactive Protein

In various embodiments, C-reactive protein (CRP) is used as a biomarker. C-reactive protein (CRP) is a non-specific acute-phase protein produced by the liver in response to tissue injury, infection, and inflammation. It increases following interleukin-6 secretion from macrophages and T cells, thus CRP and interleukin-6 (IL6) message the exact same condition/insult. CRP was so named because it was first identified as a substance in the serum of patients with acute inflammation that reacted with the C-polysaccharide of Pneumococcus. Discovered by Tillett and Francis in 1930, it was initially thought that CRP might be a pathogenic secretion since it was elevated in a variety of illnesses, including cancer. [Pepys M B, Hirschfield G M (June 2003). “C-reactive protein: a critical update”. The Journal of Clinical Investigation 111 (12): 1805-12.] The later discovery of hepatic synthesis demonstrated that it is a native protein. CRP levels rise as much as 1,000-fold after an acute event, and these high levels can be used to diagnose and monitor acute inflammatory states. Levels within the normal, non-acute-phase range (≤100 mg/L) can help assess cardiovascular risk. The high-sensitivity CRP (hs-CRP) test is used for this purpose because it can accurately determine CRP levels in the low range of 1-10 mg/L.

Mildly elevated CRP levels have been linked to increased risk for various cardiovascular-related disorders, including coronary heart disease (CHD), peripheral artery disease (PAD), incident stroke, congestive heart failure, sudden cardiac death, atrial fibrillation, and diabetes. [Greenland P, Alpert J S, Beller G A, et al. 2010 ACCF/AHA guideline for assessment of cardiovascular risk in asymptomatic adults: a report of the American College of Cardiology Foundation/American Heart Association Task Force on Practice Guidelines. J Am Coll Cardiol. 2010; 56:e50-103.] The predictive value of hs-CRP for cardiovascular events is independent of other established risk factors, including LDL-cholesterol. [Ridker P M, Rifai N, Rose L, et al. Comparison of C-reactive protein and low-density lipoprotein cholesterol levels in the prediction of first cardiovascular events. N Engl J Med. 2002; 347:1557-1565.] Mildly elevated hs-CRP levels also predict recurrent CHD events and poor prognosis in some patients, including those who have PAD or who have had a stroke or acute coronary syndrome (ACS). [Pearson T A, Mensah G A, Alexander R W, et al. Markers of inflammation and cardiovascular disease: application to clinical and public health practice: a statement for healthcare professionals from the Centers for Disease Control and Prevention and the American Heart Association. Circulation. 2003; 107:499-511.] Furthermore, in ACS patients, measurement of hs-CRP levels can improve the prediction of death or acute coronary events. [Ray K K, Cannon C P, Cairns R, et al. Prognostic utility of apoB/AI, total cholesterol/HDL, non-HDL cholesterol, or hs-CRP as predictors of clinical risk in patients receiving statin therapy after acute coronary syndromes: results from PROVE IT-TIMI 22. Arterioscler Thromb Vasc Biol. 2009; 29:424-430.]

Because hs-CRP levels are associated with cardiovascular risk, they can contribute to risk stratification. The 2013 ACC/AHA Guideline on the Assessment of Cardiovascular Risk recommends using hs-CRP testing if a risk-based treatment decision is uncertain after a quantitative risk assessment. [Goff D C Jr, Lloyd-Jones D M, Bennett G, et al. 2013 ACC/AHA guideline on the assessment of cardiovascular risk: a report of the American College of Cardiology/American Heart Association Task Force on Practice Guidelines. Circulation. 2013; November 12] If elevated, an hs-CRP level can move a patient from an intermediate risk category (as determined by traditional risk factors) into a high-risk category. [U.S. Preventive Services Task Force. Using nontraditional risk factors in coronary heart disease risk assessment: U.S. Preventive Services Task Force recommendation statement. Ann Intern Med. 2009; 151:474-482.; Emerging Risk Factors Collaboration, Kaptoge S, Di Angelantonio E, et al. C-reactive protein, fibrinogen, and cardiovascular disease prediction. N Engl J Med. 2012; 367:1310-1320.; NACB LMPG Committee Members, Myers G L, Christenson R H, et al. National Academy of Clinical Biochemistry laboratory medicine practice guidelines: emerging biomarkers for primary prevention of cardiovascular disease. Clin Chem. 2009; 55:378-384.] Approximately 10% of the men at intermediate risk could be reclassified in this manner. [U.S. Preventive Services Task Force. Using nontraditional risk factors in coronary heart disease risk assessment: U.S. Preventive Services Task Force recommendation statement. Ann Intern Med. 2009; 151:474-482] This reclassification can help the clinician decide whether to prescribe preventive therapy in borderline cases.

CRP elevation above base levels is not definitively diagnostic on its own, as it can rise in several cancers, rheumatologic, gastrointestinal, and cardiovascular conditions, and infections, not to mention acute events like trauma. And, because it is so-called “non-specific,” testing for CRP has not become a standard or recognized test in baseline health assessments. However, measuring core body temperature with a thermometer is also non-specific, yet it provides healthcare professionals a great deal of information about cause, effect, and treatment.

There is increasing evidence about inflammatory processes in the development of dementia. Therefore, inflammation has been believed to play a pivotal role in cognitive decline, Alzheimer's disease (AD), and vascular dementia. It is important to identify modifiable risk factors which could be used in preventing or delaying the onset of dementia. The result of one study suggests the presence of inflammatory activity is related with dementia, not only AD, but also vascular dementia associated with cerebrovascular disease. [Wang, Min-Jeong, et al. “A Clinical Significance of High-Sensitivity C-reactive Protein Level in Alzheimer's Disease and Vascular Dementia.” Dementia and Neurocognitive Disorders 11.4 (2012): 131-135]

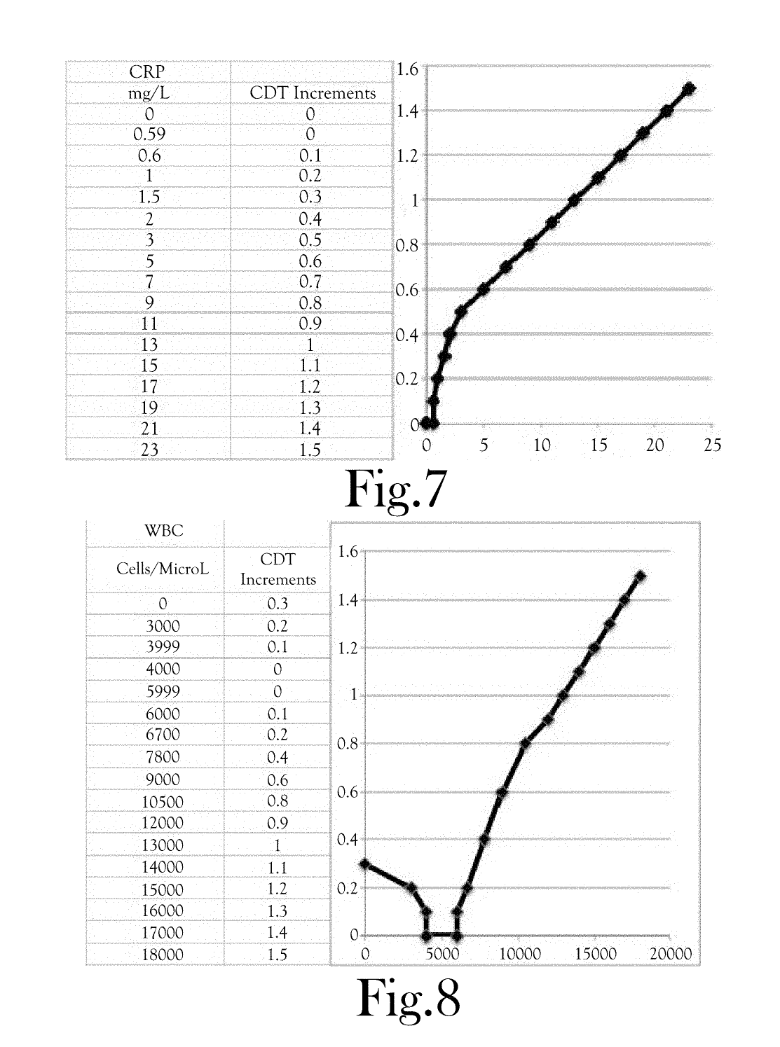

Elevated CRP is a biomarker for increased risk of premature mortality. C-reactive protein was examined in 12 studies. [Barron, Evelyn, et al. “Blood-borne biomarkers of mortality risk: systematic review of cohort studies.” PloS one 10.6 (2015): e0127550.] Meta-analysis was conducted on the relationship between CRP and mortality and FIG. 7 presents results by type of mortality. Higher CRP at baseline was significantly associated with an increased risk of all-cause mortality (HR 1.42, 1.25-1.62, p<0.0001) and cardiovascular disease (CVD) mortality (HR 1.31, 1.02-1.68, p=0.033). Higher CRP concentrations at baseline were associated with greater risk of cancer mortality (HR 1.62, 1.13-2.33, p=0.009).

Subgroup analysis by follow-up length showed that among studies with follow up of 5 years or less and studies with follow-ups over 5 years the association between CRP and all-cause mortality remained significant.

High-sensitivity cardiac CRP was able to predict risk of incident myocardial infarction, stroke, peripheral arterial disease, and sudden cardiac death among healthy individuals with no history of cardiovascular disease, as well as predict recurrent events and death in patients with acute or stable coronary syndromes. This inflammatory marker provided prognostic information that was independent of other measures of risk such as cholesterol level, metabolic syndrome, and high blood pressure. [Bassuk S S, Rifai N, Ridker P M. High-sensitivity C-reactive protein: clinical importance. Curr Probl Cardiol. 2004 August; 29(8):439-93.]

The American Heart Association and U.S. Centers for Disease Control and Prevention have defined risk groups as follows:

Low Risk: less than 1.0 mg/L

Average risk: 1.0 to 3.0 mg/L

High risk: above 3.0 mg/L

In exemplary embodiments, the average of 2 hs-CRP measurements, done 2 weeks apart, should be used when interpreting hs-CRP values in chronic disease. hs-CRP values in the range of 3.1 to 10 mg/L indicate an approximate 2-fold increased risk of CVD compared with values <1.0 mg/L. Levels persistently above 10 mg/L may indicate an acute inflammatory process; sources of infection or inflammation should be sought and the test repeated at least 2 weeks after the inflammatory response has resolved. [Pearson T A, Mensah G A, Alexander R W, et al. Markers of inflammation and cardiovascular disease: application to clinical and public health practice: a statement for healthcare professionals from the Centers for Disease Control and Prevention and the American Heart Association. Circulation. 2003; 107:499-511.] However, persistent levels about 10 mg/L indicate a subject at high risk of developing chronic disease or experiencing a sudden adverse event, including death.

In a patient with intermediate CVD risk, hs-CRP levels ≥2 mg/L support reclassifying the patient into a high-risk category. [Goff D C Jr, Lloyd-Jones D M, Bennett G, et al. 2013 ACC/AHA guideline on the assessment of cardiovascular risk: a report of the American College of Cardiology/American Heart Association Task Force on Practice Guidelines. Circulation. 2013; November 12]

A number of compounds have been shown to affect C-reactive protein concentrations in a subject. The most well established compounds include: cyclooxygenase inhibitors (aspirin, rofecoxib, celecoxib), platelet aggregation inhibitors (clopidogrel, abciximab), lipid lowering agents (statins, ezetimibe, fenofibrate, niacin, diets), beta-adrenoreceptor antagonists and antioxidants (vitamin E), as well as angiotensin converting enzyme (ACE) inhibitors (ramipril, captopril, fosinopril), reduce serum levels of CRP; while enalapril and trandolapril have not been shown to have the same effect. Angiotensin receptor blockers (ARBs) (valsartan, irbesartan, olmesartan, telmisartan) markedly reduce serum levels of CRP. The findings with other ARBs (losartan and candesartan) were inconsistent. Antidiabetic agents (rosiglitazone and pioglitazone) reduce CRP levels, while insulin is ineffective. Calcium channel antagonists have variable effects on CRP levels. Hydrochlorothiazide and oral estrogen do not affect CRP. CRP-lowering effect of statins is likely to contribute to the minutely favorable outcome of statin therapy in cardiovascular disease but the adverse impacts of these drugs leads to a null or negative benefit. The data suggest that lipid lowering agents, ACE inhibitors, ARBs, antidiabetic agents, antiinflammatory and antiplatelet agents, vitamin E, and beta-adrenoreceptor antagonists lower serum or plasma levels of CRP, while vitamin C, oral estrogen and hydrochlorothiazide do not affect CRP levels. [Prasad, Kailash. “C-reactive protein (CRP)-lowering agents.” Cardiovascular drug reviews 24.1 (2006): 33-50.]

We have found the unexpected result that elevated C-reactive protein, associated with chronic inflammation is an accurate biomarker for specific chronic diseases—thus is not “non-specific.” The etiology of the diseases overlap, thus making chronically elevated CRP appear non-specific. CRP is not the cause, but is a biomarker for disease. Thus strategies to directly and indiscriminately lower C-reactive protein is not the appropriate strategy to optimize health benefit outcomes in subjects. C-reactive protein, while correlating well with chronic disease burden, particularly cardiovascular disease is best regarded strictly as a biomarker. Therapeutic strategies we proposed are not derived around C-reactive protein lowering. Instead, further diagnostic methods must be conducted to determine the antecedents of elevated C-reactive protein. Therapy strategies must be based on these antecedent finding. Our clinical experience reveals that such strategies, defacto, result in the lowering of C-reactive protein blood levels with concomitant improvement in chronic disease morbidity and mortality.

In various embodiments, C-reactive protein contributes to a subject's chronic disease temperature as follows: Total maximum contribution to the CDT calculation is 1.5° F. (0.83° C.). See FIG. 7.

White Blood Cell Counts

White blood cells (WBC), also called leukocytes or leucocytes, are the cells of the immune system that are involved in protecting the body against both infectious disease and foreign invaders. All leukocytes are produced and derived from a multipotent cell in the bone marrow known as a hematopoietic stem cell. Leukocytes are found throughout the body, including the blood and lymphatic system. Five different and diverse types of leukocytes exist, neutrophils, eosinophils, basophils, lymphocytes, and monocytes. They aredistinguished by their physical and functional characteristics. The number of leukocytes in the blood is often an indicator of disease. The normal white cell count is usually between 4 and 11×10⁹/L. This is often expressed as 4,000-11,000 white blood cells per microliter of blood. They make up approximately 1% of the total blood volume in a healthy adult.

White blood cell elevation is more commonly associated with the following conditions: Acute lymphocytic leukemia, Acute myelogenous leukemia (AML), Allergy, especially severe allergic reactions, Chronic lymphocytic leukemia, Chronic myelogenous leukemia, Drugs, such as corticosteroids and epinephrine, Myelofibrosis, Certain bacterial infections, Certain viral infections, Polycythemia vera, Rheumatoid arthritis, Smoking, Stress, such as severe emotional or physical stress, Tuberculosis, Whooping cough. Elevated white blood cell counts is now recognized as having association with most chronic inflammatory diseases including: metabolic disorders, neurodegenerative disorders, cardiovascular disorders, and autoimmune disorders. The elevation of one or more of the various leukocyte types provides insight into the causes and disorders.

White blood cell reference ranges vary. Healthy people have a baseline level WBC count appropriate to their individual physiology and this value rises when the body of a subject goes on the defense against illness. Several labs and other authoritative sources publish different “normal” ranges. Table 7:

TABLE 7
White Blood Cell Reference Ranges

| Source | WBC (cells/milliliter) Normal Range |
|---|---|
| LabCorp | 4,500-10,000 |
| Mayo Clinic | 3,500-10,500 |
| WebMd | 5,000-10,000 |
| Quest Diagnostics | 3,800-10,800 |

In exemplary embodiments, four studies examined the association between WBC count and all-cause mortality with meta-analysis. Higher WBC count at baseline was associated with greater risk of all-cause mortality (HR 1.36, 1.13-1.64, p=0.001). [Barron, Evelyn, et al. “Blood-borne biomarkers of mortality risk: systematic review of cohort studies.” PloS one 10.6 (2015): e0127550.] WBC counts were evaluated as part of the US federally supported Women's Health Initiative. Investigators at medical centers all over the United States collected information on 72,242 postmenopausal women 50 to 79 years old. All were free of heart and blood vessel disease at the start of the study. During six years of follow-up, 1,626 heart disease deaths, heart attacks, and strokes occurred. Women with more than 6.7 billion white cells per liter of blood (6,700 cells/mL) had more than double the risk of fatal heart disease than women with 4.7 billion cells per liter or lower (4,700 cells/microliter). Leukocyte count >6.71×10⁹ cells/L is associated with an approximate 50% increase in the risk of myocardial infarction (heart attack), stroke, total vascular disease, and total mortality, independent of other risk factors. The risk of coronary death is higher, estimated as a 230% increase [http://news.harvard.edu/gazette/legacy-gazette/#, Mar. 17, 2005., Mar. 17, 2005]. Subjects with baseline WBC <3,500 cells/microliter and WBC >6,000 cells/microliter have higher mortality than those with 3,500 to 6,000 WBC/microliter. Subjects who died had higher WBC than those who survived, and the difference is statistically significant within 5 years before death. [Wheeler J. G., Mussolino M. E., Gillum R. F., Danesh J.; Associations between differential leucocyte count and incident coronary heart disease: 1764 incident cases from seven prospective studies of 30,374 individuals. Eur Heart J. 25 2004:1287-1292] Elevated WBC count in the elderly predicts survival. More than 425 swedes 75 years old participated in the study. The average WBC count for men and women in the study was 6,300 and 5,700 respectively. There was a 16% increase in mortality for men and 28% increase in mortality for women for every 1,000 increase in WBC count. [Nilsson, Göran, Pär Hedberg, and John Öhrvik. “White blood cell count in elderly is clinically useful in predicting long-term survival.” Journal of aging research 2014 (2014).] Table 8.

Among participants of the Hertfordshire Ageing Study, higher neutrophils, an important subset of white blood cells, were associated with increased mortality (HR 1.33, 1.11-1.59, p=0.002). [Baylis D, Bartlett D B, Sydall H E, Ntani G, Gale C R, Cooper C, et al. Immune-endocrine biomarkers as predictors of frailty and mortality: a 10-year longitudinal study in community-dwelling older people. AGE. 2013; 35:963-71.]

A limited set of compounds have been shown to affect elevated white blood cell concentrations in a subject. Drugs that may lower a subjects WBC count include: Antibiotics, Anticonvulsants, Anti thyroid drugs, Arsenicals, Captopril, Chemotherapy drugs, Chlorpromazine, Clozapine, Diuretics, Histamine-2 blockers, Sulfonamides, Quinidine, Terbinafine, Ticlopidine. Natural substances shown to lower WBC count in subjects include those with known immunoaugmentation benefits including, vitamin D, fish oils, magnesium, and mineral supplements.

In various embodiments, WBC contributes to a subject's chronic disease temperature as follows: Total maximum contribution to the CDT calculation is 1.5° F. (0.83° C.). See FIG. 8.

Vitamin D

In various embodiments, vitamin D is used as a biomarker for chronic disease. Vitamin D refers to a group of fat-soluble secosteroids responsible for enhancing intestinal absorption of calcium, iron, magnesium, phosphate and zinc. In humans, the most important compounds in this group are vitamin D3 (also known as cholecalciferol) and vitamin D2 (ergocalciferol). Cholecalciferol and ergocalciferol can be ingested from the diet and from supplements. Very few foods contain vitamin D; synthesis of vitamin D (specifically cholecalciferol) in the skin is the major natural source of the vitamin. Dermal synthesis of vitamin D from cholesterol is dependent on sun exposure, specifically UVB radiation. American researchers Elmer McCollum and Marguerite Davis in 1914 discovered a substance in cod liver oil which later was called “vitamin A”. [Wolf G (June 2004). “The discovery of vitamin D: the contribution of Adolf Windaus”. J Nutr 134 (6): 1299-302.] British doctor Edward Mellanby noticed dogs that were fed cod liver oil did not develop rickets and concluded vitamin A, or a closely associated factor, could prevent the disease. In 1922, Elmer McCollum tested modified cod liver oil in which the vitamin A had been destroyed. The modified oil cured the sick dogs, so McCollum concluded the factor in cod liver oil which cured rickets was distinct from vitamin A. He called it vitamin D because it was the fourth vitamin to be named.

The term “vitamin” is a misnomer for vitamin D. It is really a hormone. [McClean F C, Budy A M (Jan. 28, 1964). “Vitamin A, Vitamin D, Cartilage, Bones, and Teeth”. Vitamins and Hormones 21. Academic Press. pp. 51-52] The word “vitamin” means something our body needs that it can't make, so must be obtained from food. “D hormone” (vitamin D) is instead, an essential substance that we make in our skin from sun exposure. It is a hormone like progesterone, prednisone, estrogen, or testosterone. Hormones, including vitamin D affects multiple parts of the human body and that it is essential to every cell in the body. Vitamin D has a significant effect on the activity of 229 genes. Vitamin D status is potentially one of the most powerful selective pressures on the genome in relatively recent times. [http://www.wellcome.ac.uk/news/media-office/press-releases/2010/wtx062545.htm, Aug. 24, 2010.] Serum vitamin D levels do not indicate the amount of vitamin D stored in body tissues. Vitamin D, although not synthesized by sunlight in the winter in the northern hemisphere, is available to the body by storage in fat throughout the year, assuming adequate exposure to sunlight during summer months.

Vitamin D values are useful as a predictive biomarker for a myriad of chronic diseases. Adequate levels in humans for prevents rickets, a disease that is caused by not having enough vitamin D (vitamin D deficiency). Vitamin D supplementation is used for treating weak bones (osteoporosis), bone pain (osteomalacia), bone loss in people with a condition called hyperparathyroidism, and an inherited disease (osteogenesis imperfecta) in which the bones are especially brittle and easily broken. It is also used for preventing falls and fractures in people at risk for osteoporosis, and preventing low calcium and bone loss (renal osteodystrophy) in people with kidney failure. Vitamin D elevation and optimization is important for conditions of the heart and blood vessels, including high blood pressure and high cholesterol. It is also used for diabetes, obesity, muscle weakness, multiple sclerosis, rheumatoid arthritis, chronic obstructive pulmonary disease (COPD), asthma, bronchitis, premenstrual syndrome (PMS), and tooth and gum disease. Vitamin D therapy is useful for skin conditions including vitiligo, scleroderma, psoriasis, actinic keratosis, and lupus vulgaris. It is also used for boosting the immune system, preventing autoimmune diseases, and preventing cancer. Because vitamin D is involved in regulating the levels of minerals such as phosphorous and calcium, it is used for conditions caused by low levels of phosphorous (familial hypophosphatemia and Fanconi syndrome) and low levels of calcium (hypoparathyroidism and pseudohypoparathyroidism). Sufficient and high levels of blood vitamin D (D3) is associated with significantly reduced risk of Alzheimer's disease and other neurodegenerative diseases. Epidemiological, neuropathological, experimental, and molecular genetic evidence implicates vitamin D as a candidate in influencing susceptibility to a number of psychiatric and neurological diseases. The strength of evidence varies for schizophrenia, autism, Parkinson's disease, amyotrophic lateral sclerosis, and Alzheimer's disease, and is especially strong for multiple sclerosis. [Deluca, G. C., et al. “The role of vitamin D in nervous system health and disease.” Neuropathology and applied neurobiology (2013)]

Higher 25 hydroxy vitamin D concentrations are protective in men with intermediate to high coronary risk scores for all-cause and cardiovascular mortality. [Wu C K, Chang M H, Lin J W, Caffrey J L, Lin Y S. Renal-related biomarkers and long-term mortality in the US subjects with different coronary risks. Atherosclerosis. 2011; 216:226-36. doi: 10.1016/j. atherosclerosis.2011.01.046 PMID: 21371709.] In a study of 18 independent randomized controlled trials, including 57 311 participants, a total of 4777 deaths from any cause occurred during a trial size-adjusted mean of 5.7 years. Daily doses of vitamin D supplements varied from 300 to 2000 IU. The trial size-adjusted mean daily vitamin D dose was 528 IU. In 9 trials, there was a 1.4- to 5.2-fold difference in serum 25-hydroxyvitamin D between the intervention and control groups. The summary relative risk for mortality from any cause was 0.93 (95% confidence interval, 0.87-0.99). There was neither indication for heterogeneity nor indication for publication biases. The summary relative risk did not change according to the addition of calcium supplements in the intervention. [Autier, Philippe, and Sara Gandini. “Vitamin D supplementation and total mortality: a meta-analysis of randomized controlled trials.” Archives of internal medicine 167.16 (2007): 1730-1737.]

Vitamin D toxicity results from taking an excessive amount of supplements (>10,000 IU/day) but is the level (>100 ng/ml) is not known to be achievable just through sun exposure. Vitamin D toxicity results in hypercalcemia, which can cause nausea, anorexia, constipation, confusion, and nephrolithiasis. Vitamin D excess is associated with an independent risk of incident atrial fibrillation [Smith, Megan B., et al. “Vitamin D excess is significantly associated with risk of atrial fibrillation.” Circulation 124.21 Supplement (2011): A14699]

Vitamin D reference ranges vary. An average of reference ranges includes the following categories: deficient <20 ng/mL; insufficient 20-<35 ng/mL; sufficient 35-<50 ng/mL. Values above 50ng/mL have historically been considered excessive, at least for rickett prevention and bone health. Quest Diagnostics uses a reference range of 20-100 ng/mL. Newest insights into the health benefits of sufficient vitamin D levels results in the ranges and categories presented in Table 9.

TABLE 9
Vitamin D Status Definitions

| Definition of Vitamin D Status | 25-Hydroxyvitamin D Levels | |
|---|---|---|
| Low | Less than 20 | ng/mL |
| Low-normal | Between 21-40 | ng/mL |
| Normal | Between 41-80 | ng/mL |
| High-normal | Between 81-100 | ng/mL |
| Excess | More than 100 | ng/mL |

[Smith, Megan B., et al. “Vitamin D excess is significantly associated with risk of atrial fibrillation.” Circulation 124.21 Supplement (2011): A14699.] In a large patient study review, including 6130 references and 28 clinical studies including 99,745 participants, high normal—(see table above) levels of serum vitamin D were associated with the following: 43% reduction in cardiometabolic disorders, 33% reduction in cardiovascular diseases, 55% reduction in type 2 diabetes, and 51% reduction in metabolic syndrome. [Parker, Johanna, et al. “Levels of vitamin D and cardiometabolic disorders: systematic review and meta-analysis.” Maturitas 65.3 (2010): 225-236]

In exemplary embodiments vitamin D values of 40 ng/mL may be considered the lower limit for good health and 100 ng/mL may be considered the upper limit for good health. Vitamin D levels are lower for skeletal disease, e.g., rickets (10 ng/mL) osteoporosis and fractures (20 ng/mL), than for severe diseases according to the following estimates: premature mortality (30 ng/mL), depression (30 ng/mL), diabetes (32 ng/mL), cardiovascular disease (32 ng/mL), respiratory infections (38 ng/mL L), and cancer (40 ng/mL).

Unexpected low levels of vitamin D have been shown to be caused by the activation of 25-hydroxy vitamin D (vitamin D) to the 1,25-dihydroxyvitamin D form. Here the activated form of vitamin D is the efficacious action of vitamin D in immunity. The activation process is often the cause for the failure of ingested vitamin D supplements in a subject to raise the serum vitamin D levels. A measurement of blood vitamin D levels, for subjects under supplementation, may reveal an underlying disease process. Those subjects with low vitamin D levels, but who appear to have adequate intakes of the substance should be tested for the activated (1,25-dihydroxy) form of vitamin D.

In various embodiments, vitamin D levels contributes to a subject's chronic disease temperature as follows: Total maximum contribution to the CDT calculation is 1.4° F. (0.78° C.). See FIG. 9.

Lipoprotein Associated Phospholipase A2 (Lp-PLA2)

Lp-PLA2 is a calcium-independent phospholipase A2 enzyme that is associated with both low-density lipoprotein (LDL) and, to a lesser extent, high-density lipoprotein (HDL) in human plasma and serum [Zalewski A, Macphee C. (2005) “Role of lipoprotein-associated phospholipase A2 in atherosclerosis.” Arterioscler Thromb Vasc Biol 25:923-931.] and is distinct from other such phospholipases such as cPLA2 and sPLA2. [Kudo, I. and M. Murakami (2002). “Phospholipase A2 enzymes.” Prostaglandins Other Lipid Mediat 68-69: 3-58.] Lp-PLA2 is produced by macrophages and other inflammatory cells and is expressed in greater concentrations in advanced atherosclerotic lesions than early-stage lesions (Hakkinen, T., J. S. Luoma, M. O. Hiltunen, C. H. Macphee, K. J. Milliner, L. Patel, S. Q. Rice, D. G. Tew, K. Karkola and S. Yla-Herttuala (1999). “Lipoprotein-associated phospholipase A(2), platelet-activating factor acetylhydrolase, is expressed by macrophages in human and rabbit atherosclerotic lesions.” Arterioscler Thromb Vasc Biol 19(12): 2909-2917.). Several lines of evidence suggest that oxidation of LDL plays a critical step in the development and progression of atherosclerosis (Witztum 1994, Chisolm and Steinberg 2000). Lp-PLA2 participates in the breakdown of oxidized LDL in the vascular wall by hydrolyzing the oxidized phospholipid, producing lysophosphatidylcholine and oxidized free fatty acids, both of which are potent pro-inflammatory products that contribute to the formation of atherosclerotic plaques. [Macphee, Moores et al. 1999, Macphee 2001, Suckling and Macphee 2002] Lp-PLA2 has demonstrated modest intra- and inter-individual variation, commensurate with other cardiovascular lipid markers and substantially less variability than high sensitivity C-reactive protein (hs-CRP). In addition, Lp-PLA2 is not elevated in systemic inflammatory conditions, and may be a more specific marker of vascular inflammation. The relatively small biological variation of Lp-PLA2 and its vascular specificity are of value in the detection and monitoring of cardiovascular risk. [Witztum, J. L. (1994). “The oxidation hypothesis of atherosclerosis.” Lancet 344(8925): 793-795.]

In various embodiment, Lp-PLA2 is used as a biomarker for chronic disease. It has been identified and verified in multiple human trials as an enzymatic activity which is an independent predictor of atherosclerotic disease progression and events in humans, including coronary heart disease, because it promotes oxidation of lipoproteins and certain fatty acids. It is available to physicians and patients as a blood test and is commonly referred to as the PLAC test. It does not actually measure or reflect the amount of atherosclerotic plaque present, only a factor affecting progression of existing atherosclerotic plaques. Lp-PLA2 is not influenced by acute illness such as colds and bacterial infections (as occurs with C-reactive protein), and thus serves as a clinically useful biomarker for risk of a cardiovascular event.

The PLAC Test for Lp-PLA2 activity measures the activity of lipoprotein-associated phospholipase A2 in a patient's blood. Lp-PLA2 is a biological marker for vascular inflammation, a condition associated with the buildup of plaque in the arteries that supply blood to the heart. Over time, this buildup can result in a narrowing of the arteries and lead to coronary heart disease (CHD). Patients with test results that show Lp-PLA2 activity greater than the level of 225 nanomoles per minute per milliliter (nmol/min/mL) are at increased risk for a CHD event. Patients with test results below this level are at decreased risk for a CHD event. Patients with test results higher than 225 nmol/min/mL had a CHD event rate of 7 percent, while patients with test results below that level had a CHD event rate of 3.3 percent. Black women experience a higher jump in the rate of CHD events compared to other patients when Lp-PLA2 levels are higher than 225 nmol/min/mL. [PLAC® Test for Lp-PLA2 Activity [package insert]. South San Francisco, Calif. Diadexus, Inc; 2015]

Lp-PLA2 is not just a passive marker of risk, but that it is actively involved in causing atherosclerotic plaque leading to acute heart attack or stroke. [Anderson J L. Lipoprotein-associated phospholipase A2: an independent predictor of coronary artery disease events in primary and secondary prevention. Am J Cardiol. 2008 Jun. 16; 101(12A):23F-33F.] The Atherosclerosis Risk in Communities (ARIC) study, which involved more than 1,300 patients showed that individuals with high levels of Lp-PLA2 have twice the risk of atherosclerotic stroke over the next six to eight years compared with individuals with normal Lp-PLA2 levels. The study also found that individuals with high levels of both C-reactive protein and Lp-PLA2 had the highest risk for future coronary events and stroke, after adjusting for traditional risk factors. [Ballantyne C M, Hoogeveen R C, Bang H, et al. Lipoprotein-associated phospholipase A2, high-sensitivity C-reactive protein, and risk for incident ischemic stroke in middle-aged men and women in the Atherosclerosis Risk in Communities (ARIC) study. Arch Intern Med. 2005 Nov. 28; 165(21):2479-84.]

Lp-PLA2 activity and mass are roughly linearly associated with each other, and there is a roughly log-linear association of Lp-PLA2 activity, thus mass, with risk of coronary heart disease and all vascular mortality, and less distinct associations with ischemic stroke and the aggregate of non-vascular mortality. [Thompson A, Gao P, Orfei L, et al; Lp-PLA2 Studies Collaboration. Lipoprotein-associated phospholipase A2 and risk of coronary disease, stroke, and mortality: collaborative analysis of 32 prospective studies. Lancet. 2010; 375 (9725):1536-1544.]

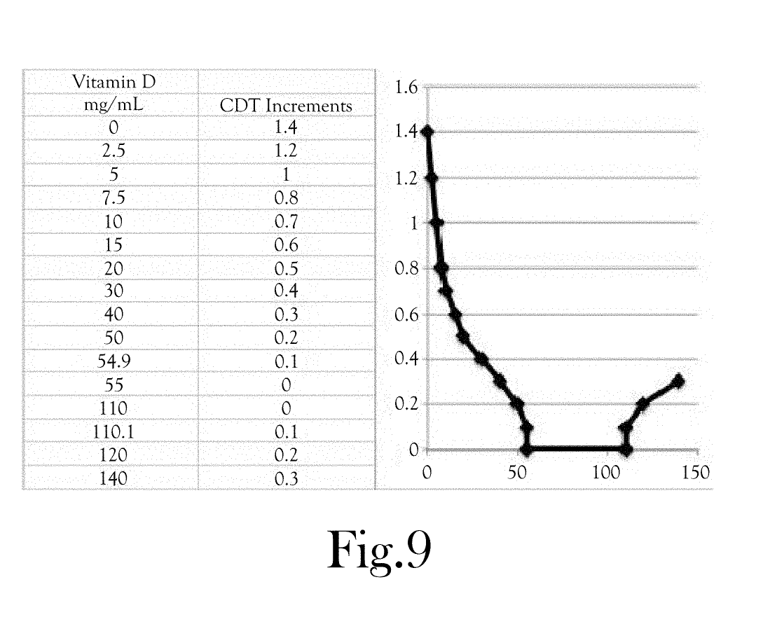

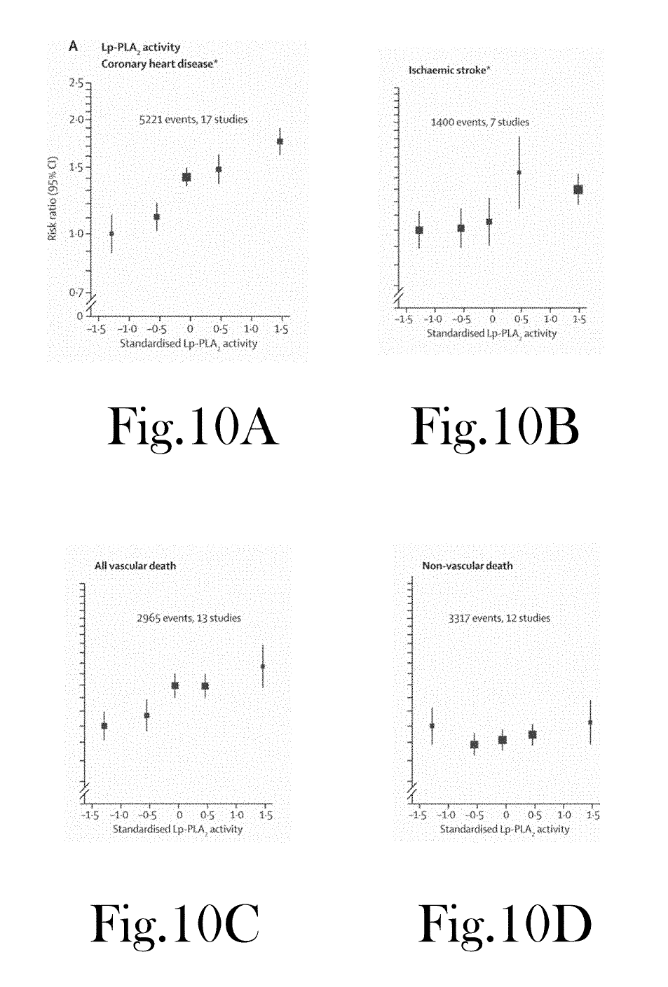

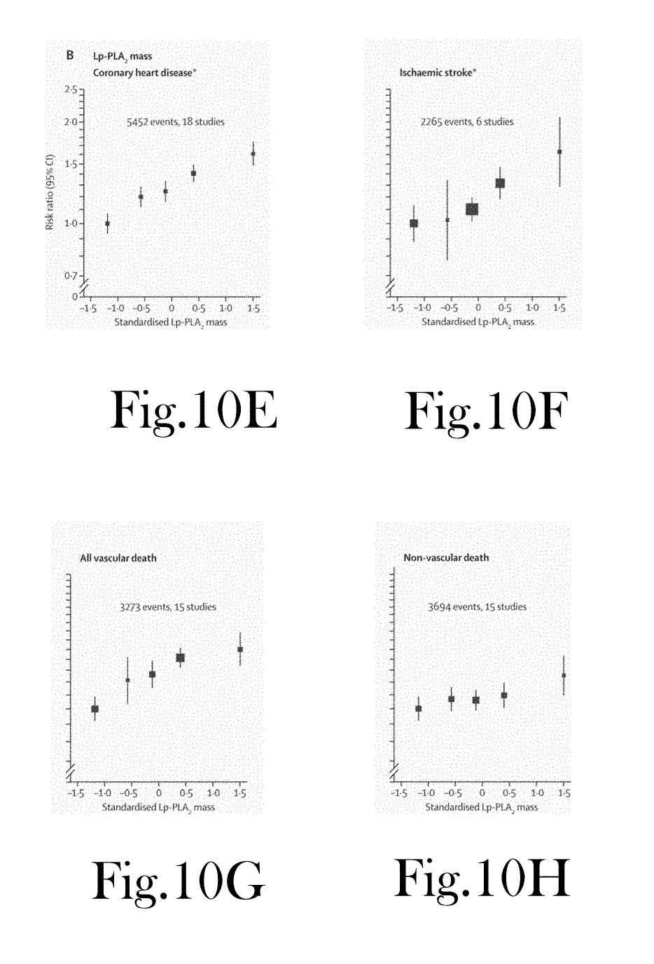

A 2008 consensus panel recommended testing Lp-PLA2 as an adjunct to traditional risk factor assessment in individuals with moderate or high risk of cardiovascular disease as defined by Framingham risk scores. The panel found that an Lp-PLA2 level >200 ng/mL indicates an individual's risk is actually higher than that determined using Framingham risk scores. [Davidson M H, Corson M A, Alberts M J, et al. Consensus panel recommendation for incorporating lipoprotein-associated phospholipase A2 testing into cardiovascular disease risk assessment guidelines. Am J Cardiol. 2008; 101(suppl):51F-57F.] Though the consensus panel only recommended Lp-PLA2 measurement in moderate- or high-risk individuals, studies have shown that elevated Lp-PLA2 also predicts coronary artery disease and ischemic stroke in the general population. [Daniels L B, Laughlin G A, Sarno M J, et al. Lipoprotein-associated phospholipase A2 is an independent predictor of incident coronary heart disease in an apparently healthy older population: The Rancho Bernardo Study. J Am Coll Cardiol. 2008; 51:913-919. Thompson A, Gao P, Orfei L, et al; Lp-PLA2 Studies Collaboration. Lipoprotein-associated phospholipase A2 and risk of coronary disease, stroke, and mortality: collaborative analysis of 32 prospective studies. Lancet. 2010; 375(9725):1536-1544.] See FIGS. 10A-H, Lp-PLA2 Activity—Morbidity, and Mortality.

Lp-PLA2 elevation is more commonly associated with the following conditions: cerebral thrombosis, first and recurrent coronary events, adverse prognosis after acute coronary syndrome, and cardiovascular disease associated with metabolic syndrome.

Lp-PLA2 references ranges are well established, and risk of disease or death increases in a log-linear manner with Lp-PLA2 activity. The preponderance of evidence suggests that a concentration <200 ng/mL is optimal, a concentration from 200-235 ng/mL is associated with a moderate risk of cardiovascular disease and stroke, and a concentration >235 ng/mL is associated with a high risk of cardiovascular disease and stroke. Risk is independent of age and gender. [Lp-PLA(2) Studies Collaboration, Thompson A, Gao P, Orfei L, et al. Lipoprotein-associated phospholipase A(2) and risk of coronary disease, stroke, and mortality: Collaborative analysis of 32 prospective studies. Lancet. 2010; 375:1536-1544.]

Predictive Lp-PLA2 levels for cardiovascular morbidity and mortality are:

Low risk: <200 ng/mL

Borderline risk: 200-235 ng/mL

High risk: >235 ng/mL.

In exemplary embodiments, Lp-PLA2 values <200 ng/mL may be considered the upper limit for good health in all people. Values of Lp-PLA2 in the range 200-235 ng/mL imply borderline or increased risk. High risk is assigned to individuals with test values >235 ng/mL. Due to the log-linear nature of risk, risk stratification above 235 ng/mL is prudent.

A limited set of compounds have been shown to affect Lp-PLA2 concentrations in a subject. Lp-PLA2 is reduced by lifestyle intervention and combination lipid-modifying therapy. The changes in Lp-PLA2 are only partially explained by the changes observed in LDL-C. Attacking the causes of inflammation appears to be the most appropriate therapeutic approach, if those causes are identifiable.

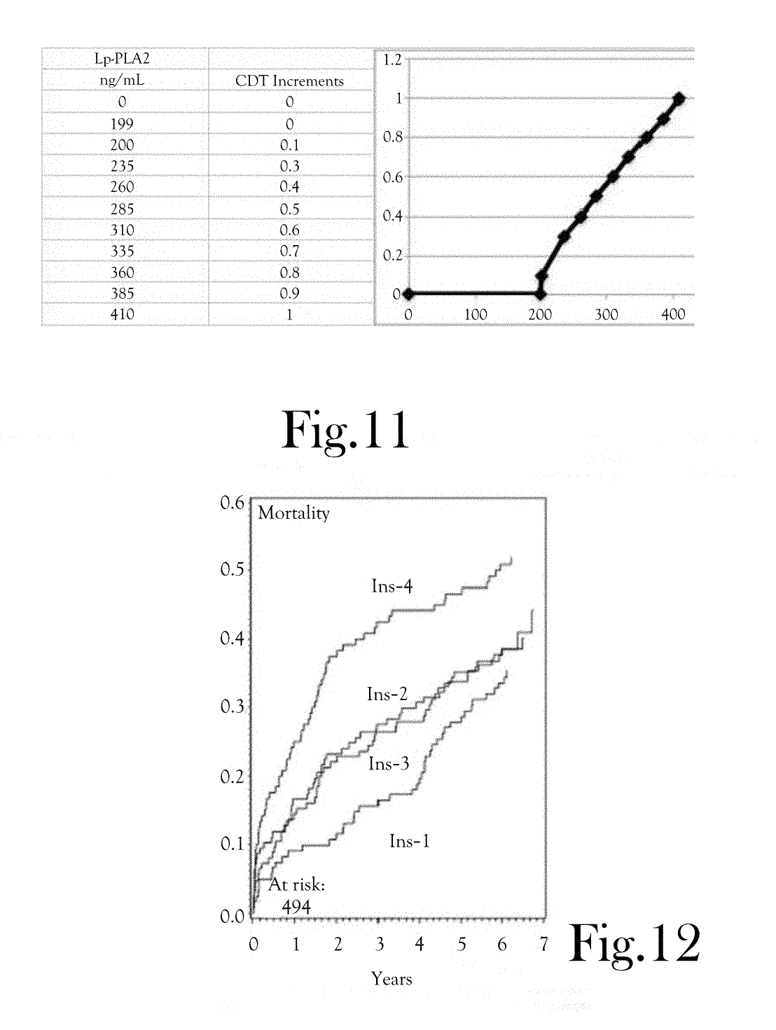

In various embodiments, Lp-PLA2 contributes to a subject's chronic disease temperature as follows: Total maximum contribution to the CDT calculation is 1.0° F. (0.56° C.). See FIG. 11.

Insulin

In various embodiments, Insulin is used as a biomarker. Insulin is an anabolic hormone that promotes glucose uptake, glycogenesis, lipogenesis, and protein synthesis of skeletal muscle and fat tissue through the tyrosine kinase receptor pathway. In addition, insulin is the most important factor in the regulation of plasma glucose homeostasis, as it counteracts glucagon and other catabolic hormones—epinephrine, glucocorticoid, and growth hormone. It has a long history of discovery starting in 1869 when pancreatic islets were first noted. Insulin is now the most widely studied of all molecules in medicine.

Insulin levels track, in a dose dependent manner, with the severity of insulin resistance as insulin resistance is compensated by the action of the brain by first regulating the pancreas to produce more insulin and then, as needed, the liver to produce more glucose. Hence the level of insulin is increased first in people pre-metabolic conditions such as type 2 diabetes. Eventually, as insulin resistance increases, blood glucose levels also rise. The elevated levels of insulin and glucose are actually protective as they make sure that the brain and other tissue in the body receive the proper energy-producing fuels for proper cellular function in compensation for the state of insulin resistance.

Insulin values are useful as a predictive biomarker for metabolic syndromes. Chronically elevated insulin is a marker of metabolic dysfunction, and typically accompanies high fat mass, poor glucose tolerance (prediabetes and diabetes) and blood lipid abnormalities. Conditions associated with increased insulin resistance (beta cell compensates via hypersecretion of insulin) include the following: Obesity, Steroid administration, Acromegaly, Cushing syndrome, Insulin receptor mutation, and Type 2 diabetes (early stage). Conditions associated with beta-cell destruction include the following: Post pancreatectomy, chronic pancreatitis, Autoimmune destruction, and Type 1 diabetes. According to the NHANES III study, metabolic disorder affects 24% of Americans. The average fasting insulin level in the U.S., according to the NHANES III survey, is 8.8 uIU/mL for men and 8.4 for women. [Nelson, Karin M., Gayle Reiber, and Edward J. Boyko. “Diet and exercise among adults with type 2 diabetes findings from the third national health and nutrition examination survey (NHANES III).” Diabetes care 25.10 (2002): 1722-1728.]

Pre-diabetes is a condition in which blood glucose levels are higher than normal, but not high enough to be classified as full-blown diabetes. However, insulin levels elevate first, and in pre-diabetics, insulin levels have risen above normal levels. Those with pre-diabetes are at increased risk of developing type 2 diabetes within a decade unless they adopt a healthier lifestyle. Diabetes is defined as having a fasting plasma blood glucose level of 126 mg/dl or greater on two separate occasions. If diabetes symptoms exist and a subject has a casual blood glucose taken at any time that is equal to or greater than 200 mg/dl, and a second test shows the same high blood glucose level, then the subject has diabetes. In general, people who have a fasting plasma blood glucose in the 100-125 mg/dl range and/or an elevation in insulin compared to normal levels are defined as having impaired fasting glucose.

An analysis of patients screened for prediabetes or diabetes mellitus using fasting insulin quartiles revealed that subjects with a value in the fourth insulin quartile were 5 times as likely to have prediabetes as subjects with an insulin value in the first quartile. Subjects who met the diagnostic criteria for diabetes mellitus were excluded. Prediabetes was defined as a fasting glucose concentration > or =100 mg/dL and < or =125 mg/dL or a 2-hour postprandial glucose concentration > or =140 mg/dL and <200 mg/dL. In a study of 965 patients, 287 (29.7%) had prediabetes. The study population primarily consisted of white, obese, female patients. A multivariate model revealed that compared with the referent lowest quartile of fasting insulin (mu=4.9[+/−SD]+/−1.2 microIU/mL), subsequent insulin quartiles increased the likelihood of identifying prediabetes (quartile 2: mu=8.0+/−0.8 microIU/mL, odds ratio [OR]=2.076, confidence interval [CI]=1.241-3.273; quartile 3: mu=12.2+/−1.7 microIU/mL, OR=3.151, CI=1.981-5.015; quartile 4: mu=25.9+/−12.4 microIU/mL, OR=5.035, CI=3.122-8.122). Older age and increased diastolic blood pressure also contributed modestly to this model. Further analysis using the area under the curve revealed that at a fasting insulin level >9.0 microIU/mL, prediabetes would be correctly identified in 80% of affected patients. Fasting insulin levels, may provide the most utility as a clinical tool because the highest quartiles suggest significantly greater likelihood of identifying prediabetes. [Johnson, Jennal, et al. “Identifying prediabetes using fasting insulin levels.” Endocrine Practice 16.1 (2009): 47-52.] Fasting serum concentrations of insulin were higher in patients with insulin resistance (16.2±5.0) than in patients without insulin resistance (7.3±2.2 IU/ml) and in controls (8.0±2.9 IU/ml). The importance of the investigation was that the subjects recruited in the study were BMI matched. [Mishima, Yasuo, et al. “Relationship between serum tumor necrosis factor-α and insulin resistance in obese men with Type 2 diabetes mellitus.” Diabetes research and clinical practice 52.2 (2001): 119-123.]

Fasting insulin level is associated with outcomes in women with early breast cancer. High levels of fasting insulin identify women with poor outcomes. Fasting insulin was associated with distant recurrence and death; the hazard ratios and 95% confidence intervals (CI) for those in the highest (>51.9 pmol/L) versus the lowest (<27.0 pmol/L) insulin quartile were 2.0 (95% CI, 1.2 to 3.3) and 3.1 (95% CI, 1.7 to 5.7), respectively. There was some evidence to suggest that the association of insulin with breast cancer outcomes may be nonlinear. Insulin was correlated with body mass index (Spearman r=0.59, P<0.001), which, in turn, was associated with distant recurrence and death (P<0.001). In multivariate analyses that included fasting insulin and available tumor- and treatment-related variables, adjusted hazard ratios for the upper versus lower insulin quartile were 2.1 (95% CI, 1.2 to 3.6) and 3.3 (95% CI, 1.5 to 7.0) for distant recurrence and death, respectively. [Goodwin, Pamela J., et al. “Fasting insulin and outcome in early-stage breast cancer: results of a prospective cohort study.” Journal of Clinical Oncology 20.1 (2002): 42-51.]

In the Homeostasis model assessment for insulin resistance (HOMA-IR) the association between fasting insulin and glucose with coronary heart disease (CHD) mortality in nondiabetic men was evaluated. Fasting insulin and fasting plasma glucose were determined to be independent risk factors for CHD mortality. Increase in risk of death is shown to become relevant at a fasting serum insulin of >9.7 mIU/L. The effect of fasting insulin by quartile is provided in Table 10.

TABLE 10
Quartiles of Fasting Serum Insulin (mU/L)

| | | |
|---|---|---|
| Q1 (2.9-7.3) | 1.00 | |
| Q2 (7.3-9.7) | 0.84 | 0.390 |

(0.57-1.25)

| | | |
|---|---|---|
| Q3 (9.7-13.1) | 1.04 | 0.818 |

(0.72-1.50)

| | | |
|---|---|---|
| Q4 (13.1-55.7) | 1.59 | 0.016 |

(1.09-2.32)

Multivariate model adjusted for age, prevalent coronary heart disease, cigarette smoking, body mass index, systolic blood pressure, serum LDL-cholesterol, plasma fibrinogen, blood leukocytes and alcohol consumption

In a study of hyperinsulinaemia, an association was established with increased long-term mortality following acute myocardial infarction in non-diabetic patients. In a univariate regression analysis, values in the upper quartile of insulin, glucose, HbA1c, and urinary albumin were associated with an excess mortality risk (RR=1.8 (1.2-2.7), p=0.002; RR=1.6 (1.2-2.1), p=0.001; RR=1.9 (1.3-2.9), p=0.001; RR=1.6 (1.2-2.1), p=0.02 respectively). However, only a high insulin level remained significant in a multivariable analysis (RR=1.54 (1.03-2.31), p=0.04) including baseline variables, left ventricular systolic function and in-hospital complications. Thus, high fasting plasma insulin is an independent risk factor of all-cause mortality in non-diabetic patients with acute myocardial infarction. Cumulative mortality from all causes stratified in quartiles of fasting plasma insulin: First: insulin <6.4 mU/l; Second: insulin 6.4-9.3 mU/l; Third: insulin 9.4-13.5 mU/l; Fourth: insulin >13.5 mU/l. [Kragelund, Charlotte, et al. “Hyperinsulinaemia is associated with increased long-term mortality following acute myocardial infarction in non-diabetic patients.” European heart journal 25.21 (2004): 1891-1897.] FIG. 12 shows the cumulative mortality from all causes stratified in quartiles of fasting plasma Insulin.

Risk of future hypertension is connected to increased levels of insulin. Data from 11,123 adults, aged 20-65 years, who had no history of hypertension or diabetes mellitus were evaluated at a 2004 medical examination in a health promotion program and had attended a repeat examination in 2008. Subjects were divided into four groups according to baseline quartiles of fasting insulin and dichotomized fasting insulin levels at baseline and after 4 years: low-low, low-high, high-low, high-high. In four years, 1142 subjects (10.3%) developed hypertension. The odds ratio (OR) for the development for hypertension increased as the quartiles of baseline fasting insulin levels and changes in fasting insulin levels increased from the first to the fourth quartile (OR 1.15, 1.35, and 1.95 vs. 1.07, 1.22, and 1.41, respectively), after adjusting for multiple factors. The OR for hypertension was 2.0-fold higher in the high-high group and 1.34-fold higher in the low-high group than in the low-low group. [Park, Se Eun, et al. “Impact of hyperinsulinemia on the development of hypertension in normotensive, nondiabetic adults: a 4-year follow-up study.” Metabolism-Clinical and Experimental 62.4 (2013): 532-538.]

Detailed measurements of fasting insulin were performed on subjects on the isolated Melanesian island of Kitava. [Lindeberg, S. Apparent absence of cerebrocardiovascular disease in Melanesians. Risk factors and nutritional considerations—the Kitava Study. 1994, University of Lund.] Measurements were also made of age-matched Swedish volunteers. In male and female Swedes, the average fasting insulin ranges from 4-11 uIU/mL, and increases with age. From age 60-74, the average insulin level is 7.3 uIU/mL. In contrast, the range on Kitava is 3-6 uIU/mL, which does not increase with age. In the 60-74 age group, in both men and women, the average fasting insulin on Kitava is 3.5 uIU/mL. Kitavans are lean and have an undetectable rate of heart attack and stroke. [Lindeberg, S, Nilsson-Ehle, P, Terént, A, Vessby, B, and Scherstén, B. Cardiovascular risk factors in a Melanesian population apparently free from stroke and ischaemic heart disease—the Kitava study. J Intern Med, 1994; 236: 331-340.] Women of the Shuar hunter-gatherers of the Amazon rainforest have an average fasting insulin concentration of 5.1 uIU/mL. [Lindgärde, Folke, et al. “Traditional versus agricultural lifestyle among Shuar women of the Ecuadorian Amazon: effects on leptin levels.” Metabolism 53.10 (2004): 1355-1358.]

Insulin levels track, in a dose dependent manner, with the severity of insulin resistance as insulin resistance is compensated by the action of the brain by first regulating the pancreas to produce more insulin and then, as needed, the liver to produce more glucose. Hence the level of insulin is increased first in people heading toward metabolic conditions such as type II diabetes. Eventually, as insulin resistance increases, blood glucose levels also rise. The elevated levels of insulin and glucose are actually protective as they make sure that the brain and other tissue in the body receive the proper energy-producing fuels for proper cellular function in compensation for the state of insulin resistance.

Insulin reference ranges vary. Quest Diagnostics reports a reference range of 2.0-19.6 μIU/mL. Melmed et al., published the following insulin values, Table 11:

TABLE 11
Insulin Reference Range Values

| | | Insulin Level |
|---|---|---|
| | Insulin Level | (SI Units*) |
| Fasting | <25 mIU/L | <174 pmol/L |
| 30 minutes after glucose | 30-230 mIU/L | 208-1597 pmol/L |
| administration | | |
| 1 hour after glucose administration | 18-276 mIU/L | 125-1917 pmol/L |
| 2 hour after glucose administration | 16-166 mIU/L | 111-1153 pmol/L |
| ≥3 hours after glucose administration | <25 mIU/L | <174 pmol/L |

*SI unit: conversional units × 6.945

[Melmed S, Polonsky K S, Larsen P R, Kronenberg H M. Williams Textbook of Endocrinology. 12th ed. Philadelphia: Elsevier Saunders; 2011.]

In exemplary embodiments, insulin values between 2 and 6 uIU/mL are within our evolutionary template with 6 uIU/mL being considered the upper limit for good health. All values elevated above 6 uIU/mL more than 3 hours after glucose administration are both indicative and predictive of a metabolic condition or a reversible sub-optimal glucose processing condition. No clear-cut dose dependent data on elevation of insulin and severity of current or future disease is apparent and consistent in the literature. However, it is reasonable to segment insulin levels into quartiles of risk, starting at a base physiologically healthy level of <=6 uIU/mL.

A new approach is emerging for controlling insulin levels in metabolic syndrome. Recent data have revealed that the plasma concentration of inflammatory mediators, such as tumor necrosis factor-α (TNF-α) and interleukin-6 (IL-6), is increased in the insulin resistant states of obesity and type 2 diabetes, raising questions about the mechanisms underlying inflammation in these two conditions. It is also intriguing that an increase in inflammatory mediators or indices predicts the future development of obesity and diabetes. Two mechanisms might be involved in the pathogenesis of inflammation. Firstly, glucose and macronutrient intake causes oxidative stress and inflammatory changes. Chronic overmacronutrition (obesity) might thus be a proinflammatory state with oxidative stress. Secondly, the increased concentrations of TNF-α and IL-6, associated with obesity and type 2 diabetes, might interfere with insulin action by suppressing insulin signal transduction. This might interfere with the anti-inflammatory effect of insulin, which in turn might promote inflammation. [Dandona, Paresh, Ahmad Aljada, and Arindam Bandyopadhyay. “Inflammation: the link between insulin resistance, obesity and diabetes.” Trends in immunology 25.1 (2004): 4-7.] Thus a limited set of compounds that manage chronic physiological inflammatory status, but not symptomatic treatment with anti-inflammatory drugs, lower insulin while being protective against insulin resistance. Fish oils, other polyunsaturated fatty acids type omega 3, magnesium, and multi-mineral supplements may reduce insulin levels and improve insulin resistance. [Albert, Benjamin B., et al. “Higher omega-3 index is associated with increased insulin sensitivity and more favorable metabolic profile in middle-aged overweight men.” Scientific reports 4 (2014).]

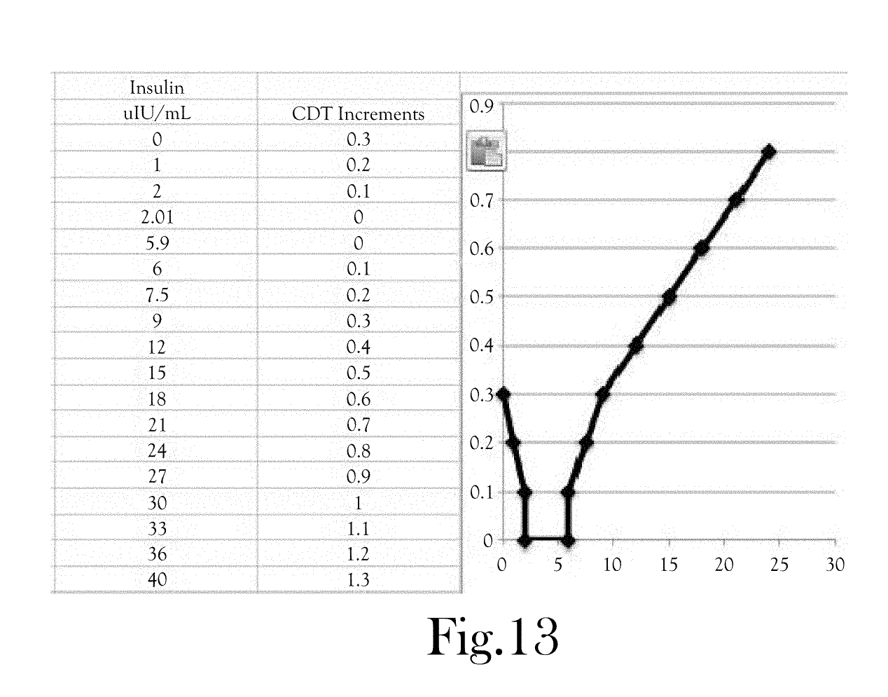

In various embodiments, fasting Insulin contributes to a subject's chronic disease temperature as follows: Total maximum contribution to the CDT calculation is 1.3° F. (0.72° C.). See FIG. 13.

F2-Isoprostanes (F2-IsoPs)

In various embodiments, F2-Isoprostanes (F2-IsoPs) is used as a biomarker. F2-IsoPs are the gold-standard for quantifying oxidative stress. The isoprostanes are prostaglandin-like compounds formed in vivo from the free radical-catalyzed peroxidation of essential fatty acids (primarily arachidonic acid) without the direct action of cyclooxygenase (COX) enzymes. The compounds were discovered in 1990 by L. Jackson Roberts and Jason D. Morrow in the Division of Clinical Pharmacology at Vanderbilt University. These nonclassical eicosanoids possess potent biological activity as inflammatory mediators that augment the perception of pain. These compounds are accurate markers of lipid peroxidation in both animal and human models of oxidative stress.

Oxidative stress and damage has been implicated in the pathogenesis of many chronic progressive diseases, such as cancer, inflammation, and neurodegenerative disorders. And there has been considerable interest in the role of oxidative stress in vascular disease as well. This interest has been driven by a wealth of data indicating that LDL oxidation is a prominent feature of atherosclerosis. [Witztum J L, Steinberg D. Role of oxidized low density lipoprotein in atherogenesis. J Clin Invest. 1991; 88: 1785-1792.] Studies have also suggested that oxidative stress is a feature of many risk factors for premature atherosclerosis, such as diabetes, [Gopaul N K, Änggård E E, Mallet A I, Betteridge D J, Wolff S P, Nourooz-Zadeh J. Plasma 8-epi-PGF2alpha levels are elevated in individuals with non-insulin dependent diabetes mellitus. FEBS Lett. 1995; 368: 225-229] hypertension, [Griendling K K, Sorescu D, Ushio-Fukai M. NAD(P)H oxidase: role in cardiovascular biology and disease. Circ Res. 2000; 86: 494-501.] and smoking. [L Morrow J D, Frei B, Longmire A W, Gaziano J M, Lynch S M, Shyr Y, Strauss W E, Oates J S, Roberts L J. Increase in circulating products of lipid peroxidation (F2-isoprostanes) in smokers: smoking as a cause of oxidative damage. N Engl J Med. 1995; 332: 1198-1203.]

F2-IsoPs are increased in cerebrospinal fluid (CSF), blood, and urine of patients with a clinical diagnosis of Alzheimer's disease (AD). These levels are highly correlated with other biomarkers of AD pathology and with the severity of the disease. And individuals with mild cognitive impairment (MCI) progress to AD at approximately 12% per year, thus MCI sufferers are believed to be at high risk to progress to a clinical diagnosis of AD. Individuals with MCI have increased brain oxidative damage before the onset of symptomatic dementia. Measurement of F2-IsoPs in a subgroup of patients with MCI have significantly higher levels in cerebrospinal fluid, plasma, and urine when compared with cognitively normal elderly subjects. [Pratico, Domenico, et al. “Increase of brain oxidative stress in mild cognitive impairment: a possible predictor of Alzheimer disease.” Archives of Neurology 59.6 (2002): 972-976.]

F2-Isoprostane concentrations in cerebrospinal fluid are elevated early in the course of dementia, and correlate with disease severity and progression. F2-Isoprostanes are elevated in urine in young patients with Down's syndrome, which is associated with precocious Alzheimer's disease-like pathology and dementia. [Praticò, D., Iuliano, L., Amerio, G., Tang, L. X., Rokach, J., Sabatino, G., Violi, F. (2000) Down's syndrome is associated with increased 8,12-iso-iPF2α-VI levels: evidence for enhanced lipid peroxidation in vivo. Ann. Neurol. 48,795-798] Pericardial F2-isoprostane concentrations increase with the functional severity of heart failure and are associated with ventricular dilatation, suggesting a possible role for in vivo oxidative stress on ventricular remodeling and the progression to heart failure.

Telomeres are nucleoprotein structures, located at the ends of chromosomes and are subject to shortening at each cycle of cell division. They prevent chromosomal ends from being recognized as double strand breaks and protect them from end to end fusion and degradation. Telomeres consist of stretches of repetitive DNA with a high G-C content and are reported to be highly sensitive to damage induced by oxidative stress. The resulting DNA strand breaks can be formed either directly or as an intermediate step during the repair of oxidative bases. In contrast to the majority of genomic DNA, there is evidence that telomeric DNA is deficient in the repair of single strand breaks. Since chronic oxidative stress plays a major role in the pathophysiology of several chronic inflammatory diseases, it is hypothesized that telomere length is reducing at a faster rate during oxidative stress. Therefore, assessment of oxidative stress may be a useful biomarker of disease progression. [Houben, Joyce M J, et al. “Telomere length assessment: biomarker of chronic oxidative stress.” Free Radical Biology and Medicine 44.3 (2008): 235-246.]

Despite the importance of measuring lipid peroxidation to explore the potential role of oxidative stress in the pathogenesis of human diseases, no previously existing assay of lipid peroxidation, prior to F2-IsoPs, was considered “ideal.” Assays that had been developed had several shortcomings related to (i) the specificity of the assay itself for the product of lipid peroxidation being measured, (ii) the product being measured was not a specific product of lipid peroxidation, (iii) the lack of sufficient sensitivity to detect levels of the product being measured in normal subjects, thus allowing the definition of a normal range, (iv) levels of the product being measured being influenced by external factors, such as the lipid content of the diet, or (v) the assay being too invasive for human investigation.

The most widely used test for oxidative stress is measurement of malondialdehyde (MDA), a product of lipid peroxidation, by a thiobarbituric acid-reacting substances (TBARS) assay. However, the use of this assay to assess oxidative stress status is problematic because MDA is not a specific product of lipid peroxidation and the TBARS assay is not specific for MDA. Another method of assessing lipid peroxidation in vivo is measurement of exhaled volatile alkanes, such as ethane and pentane. However, the accuracy of exhaled pentane as a marker of endogenous lipid peroxidation has been questioned: these hydrocarbon gases are minor end-products of peroxidation and their concentrations are influenced by the breakdown rate of peroxides. Various methods have been used to measure lipid hydroperoxides, but marked inconsistencies have been found with levels detected, for example, in human plasma, raising questions regarding accuracy of assay methodology. Lipid hydroperoxydes cannot not be detected in the circulation even under conditions of severe oxidative stress using a highly accurate and sensitive gas chromatography/mass spectrometry (GC/MS) assay, rendering this approach for assessing oxidative stress status in humans of little or no value. [Montuschi, Paolo, Peter J. Barnes, and L. Jackson Roberts. “Isoprostanes: markers and mediators of oxidative stress.” The FASEB Journal 18.15 (2004): 1791-1800.]

There are several favorable attributes that make measurement of F2-IsoPs attractive as a reliable indicator of oxidative stress in vivo: (i) F2-IsoPs are specific products of lipid peroxidation; (ii) they are stable compounds; (iii) levels are present in detectable quantities in all normal biological fluids and tissues, allowing the definition of a normal range; (iv) their formation increases dramatically in vivo in a number of animal models of oxidant injury; (v) their formation is modulated by antioxidant status; and (vi) their levels are not effected by lipid content of the diet. Measurement of F2-IsoPs in plasma can be utilized to assess total endogenous production of F2-IsoPs whereas measurement of levels esterified in phospholipids can be used to determine the extent of lipid peroxidation in target sites of interest. (vii) F2-isoprostanes are advantageous over other markers of lipid peroxidation due to their in vivo and in vitro stability and are detectable in a variety of human tissues and biological fluids including plasma, urine, lavage fluid, RBCs, and cerebrospinal fluid. Quantitation of F2-isoprostanes in a random urine specimen is considered to be the most accurate and robust measurement of circulating isoprostanes and is a noninvasive method of assessment. In addition an assay for a urinary metabolite of F2-IsoPs exists, which provides a valuable noninvasive integrated approach to assess total endogenous production of F2-IsoPs.

The relationship between total plasma concentrations of homocysteine and F2-IsoPs has been explored. Plasma concentrations of F2-IsoPs increased linearly across quintiles of homocysteine levels. The simple correlation coefficient for association between plasma concentrations of homocysteine and F2-IsoPs was 0.40 (p<0.0001). [Voutilainen S., Morrow J. D., Roberts L. J., Alfthan G., Alho H., Nyyssonen K., Salonen J. T. Enhanced in vivo lipid peroxidation at elevated plasma total homocysteine levels. Arterioscler. Thromb. Vasc. Biol. 1999; 19:1263-1266.]

Reference Values

F2-Isoprostanes reference values are not well established in the current medical paradigm and among clinical laboratories. An example of a published range is:

or =18 years: < or =1.0 ng/mg creatinine

<18 years: not established

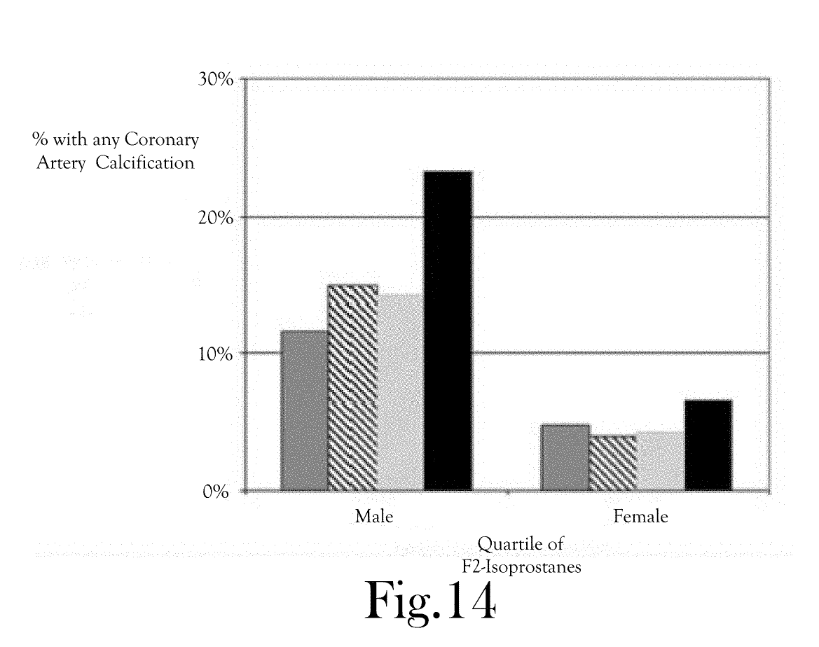

In exemplary embodiments, in the CARDIA Study, the association between increased concentrations of circulating F2-isoPs and coronary artery calcification (CAC) was demonstrated to be logarithmic. The key conclusion was a strong association between increased concentrations of circulating F2-isoprostances and coronary artery calcification in young healthy adults supporting existing data is that oxidative damage is involved in the early development of atherosclerosis. [Gross, Myron, et al. “Plasma F2-isoprostanes and coronary artery calcification: the CARDIA Study.” Clinical chemistry 51.1 (2005): 125-131.] FIG. 14 illustrates the step-up in risk of disease with F2-isoprostanes level in serum.

Within each sex group, individuals with the highest F2-isoprostane concentrations had the highest observed prevalence of CAC with increases more apparent for men than women. In men, CAC prevalence increased from quartile 1 to quartiles 2 and 3, followed by a somewhat larger increase between quartiles 3 and 4. In women, the F2-isoprostane concentrations remained relatively flat across quartiles 1-3, followed with an increase in quartile 4.

Plasma levels of F2-IsoPs measured in the diabetic patients (33.4±4.8 pg/mL, mean±SEM) were found to be significantly increased compared with levels measured in the nondiabetic patients (22.2±1.9 pg/mL) (p<0.02). Plasma F2-IsoP concentrations were found to be increased by 34% in acute hyperglycemia and this is similar to other models of oxidative damage. [Kaviarasan, Subramanian, et al. “F2-isoprostanes as novel biomarkers for type 2 diabetes: a review.” Journal of clinical biochemistry and nutrition 45.1 (2009)]

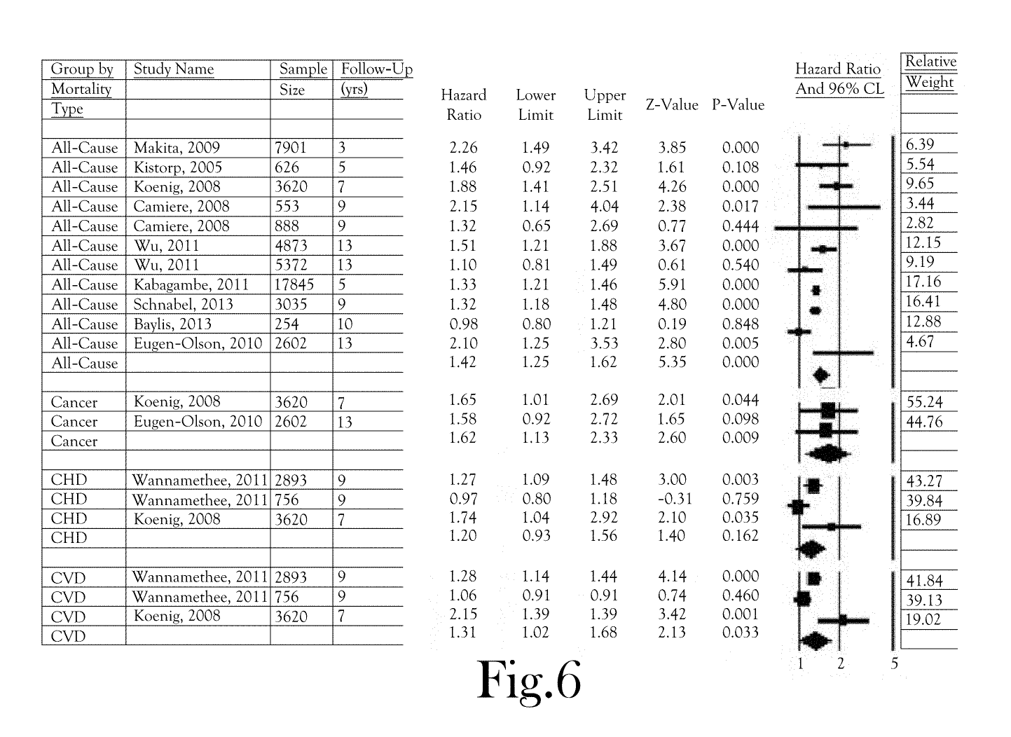

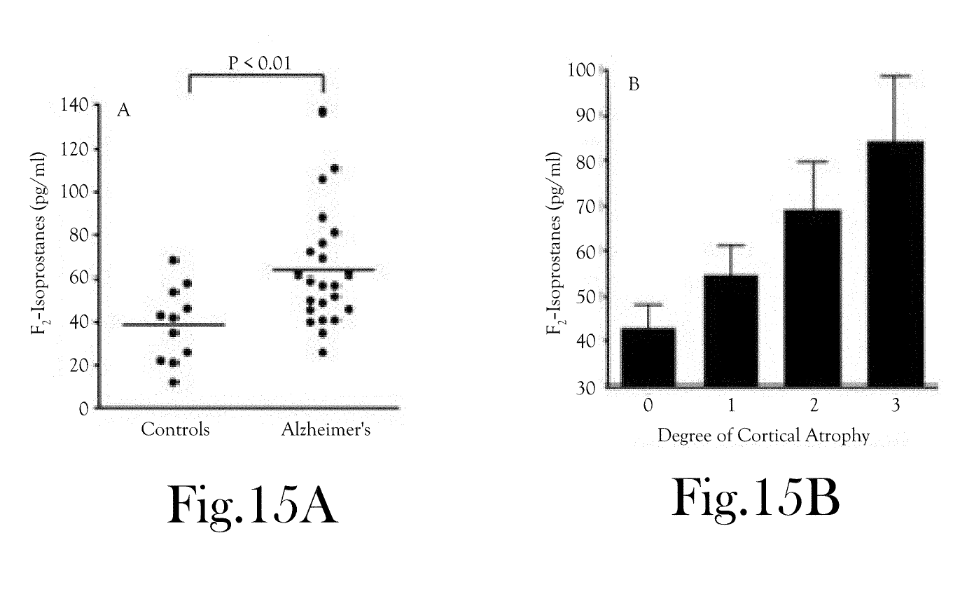

Postmortem ventricular fluid obtained from 23 patients with Alzheimer's disease and 11 age-matched controls shows significant changes in F2-isoprostanes. In FIG. 6 below, Horizontal lines are means (upper panel). F2-Isoprostane levels were significantly higher in patients with Alzheimer's disease than controls (P<0.01). Mean F2-isoprostane concentrations (±SE) in ventricular fluid were plotted against cortical atrophy in the same patients and control subjects (lower panel). Cortical atrophy was graded as absent (degree 0, n=15), mild (degree 1, n=8), moderate (degree 2, n=8), or severe (degree 3, n=4) in all patients with Alzheimer's disease and controls. Spearman's ranked correlation gave P<0.01. Analysis restricted to Alzheimer's disease patients only was statistically significant (n=23, P<0.05). [Montuschi, Paolo, Peter J. Barnes, and L. Jackson Roberts. “Isoprostanes: markers and mediators of oxidative stress.” The FASEB Journal 18.15 (2004): 1791-1800.] FIGS. 15A and 15B show the concentration of F2-isoprostanes with respect to Alzheimer's disease and the degree of Cortical Atrophy.

Strategies exist for reducing or preventing the generation of oxidative stress, thus lower or prevent the rise of F2-isoprostanes. The reduction of oxidative stress may be achieved in three levels: by lowering exposure to environmental pollutants with oxidizing properties, by increasing levels of endogenous and exogenous antioxidants, or by lowering the generation of oxidative stress by stabilizing mitochondrial energy production and efficiency. Endogenous oxidative stress could be influenced in two ways: by prevention of ROS formation or by quenching of ROS with antioxidants. However, the results of epidemiological studies where people were treated with synthetic antioxidants are inconclusive and often opposite to that expected due to the indiscriminate scavenging of detrimental and beneficial free radicals. Recent evidence suggests that antioxidant supplements do not offer sufficient protection against oxidative stress, oxidative damage or increase the lifespan of humans. The key to the future success of decreasing oxidative-stress-induced damage should thus be the suppression of oxidative damage without disrupting the well-integrated antioxidant defense network. Approach to neutralize free radicals with antioxidants should be changed into prevention of free radical formation. The best way to achieve this is through an anti-inflammatory, not an anti-oxidant strategy. [Poljsak, B. “Strategies for reducing or preventing the generation of oxidative stress.” Oxidative medicine and cellular longevity 2011 (2011).]

In exemplary embodiments, F2-isoprostane concentrations <30 pg/mL may be considered the upper limit for good health in most people. This is based on an increased risk of diabetes. Risk increases linearly in mild cognitive impairment, dementias, and Alzheimer's disease with elevation of F2-isoprostane when measured in pg/mL. Increasing risk is non-linear for coronary artery calcification in younger people and, for this indication, the correlation is less well substantiated for women.

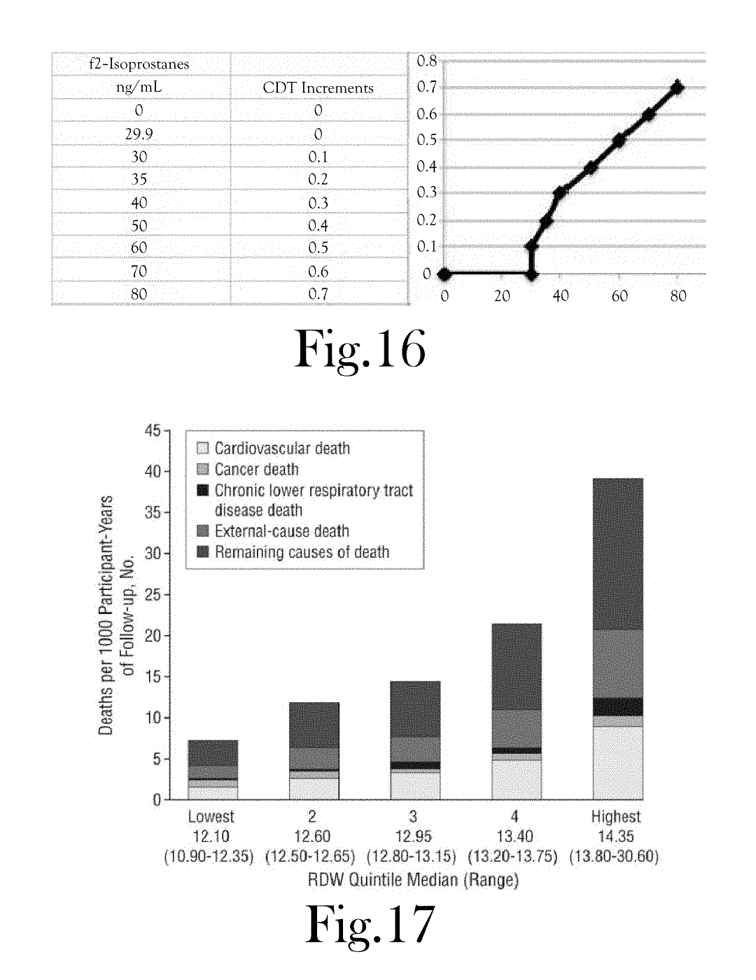

In various embodiments, F2-isoprostane contributes to a subject's chronic disease temperature as follows: Total maximum contribution to the CDT calculation is 0.7° F. (0.39° C.). See FIG. 16.

Red Blood Cell Distribution Width (RDW)

In various embodiments, red blood cell distribution width is used as a biomarker. Red blood cell distribution width (RDW or RDW-CV or RCDW or RDW-SD) is a measure of the range of variation of red blood cell (RBC) volume that is reported as part of a standard complete blood count. Usually red blood cells are a standard size of about 6-8 μm in diameter. This is a standard reported measure on a complete blood count lab test. It measures the variability in red blood cell size. In the normal state, red blood cells are continually being produced and removed from the blood at a steady rate. The young, immature red blood cells are larger than mature red blood cells. There are predictable proportions of large and small red blood cells, which can be plotted on a graph as the normal values. In certain diseases, including anemia, the RDW may be higher than normal because there are more immature or abnormal red blood cells skewing the statistical range of values. The RCDW result is nonspecific, as are most chronic diseases.

RCDW values are useful as a predictive biomarker for a variety of diseases, therefore it is a predictive biomarker of declining health, morbidity and mortality. A pubmed search including the term “red blood cell distribution width,” in the “title only” yielded 349 articles in 2014. Many of the articles discussed the association between RCDW and disease. About 42% of the articles tie abnormal RCDW and cardiovascular diseases and 15% associated abnormal RCDW with early mortality, Table 12. This table shows that this test has specificity for cardiovascular disease risk and that, when RCDW is abnormal, many diseases associated with the vascular system may matriculate in a human. This table further illustrates the connectivity of chronic diseases.

TABLE 12
Abnormal Red Blood Cell Distribution Width and Disease.

| Disease or Indication | % Articles |
|---|---|
| Mortality (all cause) | 14.90% |
| Cardiovascular Diseases | |
| Cardiovascular disease (non-specific) | 14.90% |
| Heart Failure | 7.21% |
| Heart attack | 4.81% |
| Acute coronary artery syndrome | 4.33% |
| Stroke | 3.85% |
| Thrombocytopenia | 2.88% |
| Hypertension | 2.40% |
| Atrial fibrillation | 0.96% |
| Carotid artery atherosclerosis | 0.48% |
| Total - Cardiovascular Diseases | 41.82% |
| Anemia | 11.54% |
| Metabolic syndrome | 3.85% |
| Inflammation | 3.37% |
| Iron deficiency | 3.37% |
| Kidney function | 2.40% |
| Liver disease | 1.92% |
| Rheumatoid arthritis | 0.96% |
| Cancer | 0.96% |
| Acute infection | 0.96% |
| TSH - thyroid function | 0.96% |
| Sepsis | 0.96% |
| Poor functional status | 0.96% |
| Brain injury/head trauma | 0.96% |
| COPD | 0.96% |
| Dyspnea (shortness of breath) | 0.96% |
| Blood (hematologic disease) | 0.48% |
| Microcytosis | 0.48% |
| Capillary velocity | 0.48% |
| Tuberculosis | 0.48% |
| Hematuria (blood in urine) | 0.48% |
| Hepatitis B | 0.48% |
| Bone marrow stimulation | 0.48% |
| Membrane integrity | 0.48% |
| Lupus erythematosus | 0.48% |
| HIV | 0.48% |
| Vitamin B12 deficiency | 0.48% |
| Obstructive sleep apnea | 0.48% |
| Crohn's disease | 0.48% |
| Ulcerative colitis | 0.48% |
| Smoking | 0.48% |
| Lung cancer | 0.48% |
| Acute appendicitis | 0.48% |

Red blood cell distribution width levels track, in a width dependent manner, with the severity of chronic disease. Morbidity and mortality risk associated with RCDW in the highest quintile, (≤12.5, 12.6-13.0, 13.1-13.5, 13.6-14.3, ≥14.4 “was similar in magnitude to that of being 80 years of age or older and stronger than hematocrit, platelets, or white blood cell count.” [Horne, Benjamin D., et al. “Exceptional mortality prediction by risk scores from common laboratory tests.” The American journal of medicine 122.6 (2009): 550-558.] The increase in risk follow a roughly log-linear relationship.

RCDW values are useful as a predictive biomarker of premature mortality. Higher RCDW is associated with increased mortality risk based on a large, community-based sample. Estimated mortality rates increased 5-fold from the lowest to the highest quintile of RCDW after accounting for age and 2-fold after multivariable adjustment (Ptrend <0.001 for each). A 1-SD increment in RDW (0.98%) was associated with a 23% greater risk of all-cause mortality (hazard ratio [HR], 1.23; 95% confidence interval [CI], 1.18-1.28) after multivariable adjustment. The RDW was also associated with risk of death due to cardiovascular disease, FIG. 7. (HR, 1.22; 95% CI, 1.14-1.31), cancer (1.28; 1.21-1.36), and chronic lower respiratory tract disease (1.32; 1.17-1.49). [Perlstein, Todd S., et al. “Red blood cell distribution width and mortality risk in a community-based prospective cohort.” Archives of internal medicine 169.6 (2009): 588-594.] FIG. 17 shows Red Blood Cell Distribution Width and Mortality.

It is both interesting and unusual to see one biomarker, in this case RCDW, associated with both cancer and cardiovascular disease. This type of clear correlation hints at the possibility that the causes of these two diseases overlap.

Red blood cell distribution width values are useful as a predictive biomarker for inflammation. The association of RCDW with mortality risk may be due to underlying inflammation, as inflammation is increasingly appreciated to contribute to the pathogenesis of chronic disease. Data supports an association of anisocytosis with inflammation, and suggest that the association of RCDW with mortality risk may in part be due to an effect of inflammation on both anisocytosis and risk. [Perlstein, Todd S., et al. “Red blood cell distribution width and mortality risk in a community-based prospective cohort.” Archives of internal medicine 169.6 (2009): 588-594.]

The reference range for RCDW is as follows:

RDW-SD 39-46 fL [Briggs C, Bain B J. Basic Haematological Techniques. In: Bain B J, Bates I, Laffan M, Lewis S M. Dacie and Lewis Practical Haematology. 11th ed. Philadelphia, Pa. Churchill Livingstone/Elsevier]

RDW-CV 11.6-14.6% in adult [Vajpayee N, Graham S S, Bem S. Basic Examination of Blood and Bone Marrow. In: McPherson R A, Pincus M R. Henry's Clinical Diagnosis and Management by Laboratory Methods. 22nd. Elsevier/Saunders: Philadelphia, Pa.; 2011:30.]

In exemplary embodiments, RCDW values <12.5% may be considered the upper limit for good health in all people. This is based on an increased risk of a broad range of morbidities and on increased relative and absolute mortality rates. The risk of both morbidity and mortality increases in a log-linear fashion with quintiles defined as ≤12.5, 12.6-13.0, 13.1-13.5, 13.6-14.3, ≥14.4, expressed in percent.

In various embodiments, red blood cell distribution width contributes to a subject's chronic disease temperature as follows: Total maximum contribution to the CDT calculation is 1.4° F. (0.78° C.). See FIG. 18.

Glycated Hemoglobin (HbA1C)

In various embodiments, HbA1c is used as a biomarker. HbA1c is a term commonly used in relation to diabetes. The term HbA1c refers to glycated hemoglobin. It develops when hemoglobin, a protein within red blood cells that carries oxygen throughout your body, joins with glucose in the blood, becoming ‘glycated’. When the human body processes sugar, glucose in the bloodstream naturally attaches to hemoglobin. The amount of glucose that combines with this protein is directly proportional to the total amount of sugar that is in your system at that time. Because red blood cells in the human body survive for 8-12 weeks before renewal, measuring glycated hemoglobin (or HbA1c) can be used to reflect average blood glucose levels over that duration, providing a useful longer-term gauge of blood glucose control. HbA1c was discovered in the late 1960s and its use as marker of glycemic control has gradually increased over the course of the last four decades. Recognized as the gold standard of diabetic survey, this parameter was successfully implemented in clinical practice in the 1970s and 1980s and internationally standardized in the 1990s and 2000s. The use of standardized and well-controlled methods, with well-defined performance criteria, has recently opened new directions for HbA1c use in patient care, e.g., for diabetes diagnosis. Insulin resistance and concomitant hyperinsulinemia are presented in chronic kidney disease patients without clinical diabetes and the risk increases with degree of metabolic syndrome as measure by HbA1c, Table 13. [Chen, Jing, et al. “Insulin resistance and risk of chronic kidney disease in nondiabetic US adults.” Journal of the American Society of Nephrology 14.2 (2003): 469-477.]

TABLE 13
Prevalence of chronic kidney disease (GFR < 60 ml/min per 1.73
m²) according to quartiles of glucose, insulin, C-peptide, HbA1c,
and HOMA-insulin resistance among 6453 persons without diabetes

| | No. of Cases/ | | |
|---|---|---|---|
| | Participants | % (SE) | P |
| Plasma glucose, mg/dl | | | |
| <88.9 | 20/1615 | 0.7 (0.2) | <0.001 |
| 88.9 to 95.1 | 38/1613 | 1.2 (0.3) | |
| 95.2 to 101.9 | 54/1633 | 2.2 (0.5) | |
| ≥102.0 | 73/1592 | 3.9 (0.6) | |
| Serum insulin, μU/ml | | | |
| <6.61 | 30/1604 | 0.8 (0.2) | <0.001 |
| 6.62 to 9.08 | 49/1601 | 1.8 (0.4) | |
| 9.09 to 12.88 | 49/1599 | 2.2 (0.5) | |
| ≥12.89 | 56/1597 | 3.6 (0.7) | |
| Serum C-peptide, μmol/ml | | | |
| <0.403 | 11/1609 | 0.3 (0.1) | <0.001 |
| 0.404 to 0.636 | 18/1602 | 0.6 (0.2) | |
| 0.637 to 0.937 | 46/1606 | 1.6 (0.3) | |
| ≥0.938 | 108/1601 | 5.8 (0.8) | |
| HbA1c, % | | | |
| <5.0 | 21/1913 | 0.5 (0.1) | <0.001 |
| 5.1 to 5.3 | 30/1614 | 1.2 (0.3) | |
| 5.4 to 5.6 | 43/1434 | 2.2 (0.4) | |
| ≥5.7 | 91/1470 | 6.3 (0.9) | |
| HOMA-insulin resistance | | | |
| <1.493 | 31/1600 | 0.9 (0.2) | <0.001 |
| 1.493 to 2.147 | 44/1599 | 1.4 (0.3) | |
| 2.148 to 3.153 | 47/1599 | 2.0 (0.4) | |
| ≥3.154 | 62/1599 | 4.1 (0.8) | |

Elevated HbA1c is associated with increased morbidity and mortality even in patients not diagnosed with diabetes. Mean glycaemia and HbA1c show consistent and stronger associations with cardiovascular disease risk factors than fasting glucose or postprandial glucose levels or measures of glucose variability in patients with diabetes. [Borg, R., et al. “HbA1c and mean blood glucose show stronger associations with cardiovascular disease risk factors than do postprandial glycaemia or glucose variability in persons with diabetes: the A1C-Derived Average Glucose (ADAG) study.” Diabetologia 54.1 (2011): 69-72.] In a study of more than 8000 subjects over 7 years patients who progressed to chronic kidney disease had higher mean HbA1c (7.8±1.3% vs 7.4±1.2%, p<0.001) and SD (1.0±0.8% vs 0.8±0.6%, p<0.001) than nonprogressors. Similarly, patients who developed cardiovascular disease had higher mean HbA1c (7.7±1.3% vs 7.4±1.2%, p<0.001) and SD (1.4±1.1% vs 1.1±0.8%, p<0.001) than patients who did not develop cardiovascular disease. By using multivariate-adjusted Cox regression analysis, adjusted SD was associated with incident chronic kidney disease and cardiovascular disease with corresponding hazard ratios of 1.16 (95% CI 1.11-1.22), p<0.001) and 1.27 (95% CI 1.15-1.40, p<0.001), independent of mean HbA1c and other confounding variables. [Luk, Andrea O Y, et al. “Risk association of HbA1c variability with chronic kidney disease and cardiovascular disease in type 2 diabetes: prospective analysis of the Hong Kong Diabetes Registry.” Diabetes/metabolism research and reviews 29.5 (2013): 384-390.]

In peripheral arterial disease, a positive, graded, and independent association between HbA1C and the disease is demonstrated in the following tertiles: <5.9%; 6.0-7.4%; and >7.5%. [Selvin, Elizabeth, et al. “HbA1c and peripheral arterial disease in diabetes the Atherosclerosis Risk in Communities study.” Diabetes care 29.4 (2006): 877-882.] Mean glycaemia and HbA1c show strong and consistent associations with cardiovascular risk factors and these associations are strong compared to fasting glucose and most measures of postprandial glucose and glucose variability. [Borg, R., et al. “HbA1c and mean blood glucose show stronger associations with cardiovascular disease risk factors than do postprandial glycaemia or glucose variability in persons with diabetes: the A1C-Derived Average Glucose (ADAG) study.” Diabetologia 54.1 (2011): 69-72.] Subjects who progressed to chronic kidney disease had higher mean HbA1c (7.8±1.3% vs 7.4±1.2%, p<0.001) and standard deviation (SD) (1.0±0.8% vs 0.8±0.6%, p<0.001) than nonprogressors. Similarly, patients who developed cardiovascular disease had higher mean HbA1c (7.7±1.3% vs 7.4±1.2%, p<0.001) and SD (1.4±1.1% vs 1.1±0.8%, p<0.001) than patients who did not develop cardiovascular disease. By using multivariate-adjusted Cox regression analysis, adjusted SD was associated with incident chronic kidney disease and cardiovascular disease with corresponding hazard ratios of 1.16 (95% CI 1.11-1.22), p<0.001) and 1.27 (95% CI 1.15-1.40, p<0.001), independent of mean HbA1c and other confounding variables. [Luk, Andrea O Y, et al. “Risk association of HbA1c variability with chronic kidney disease and cardiovascular disease in type 2 diabetes: prospective analysis of the Hong Kong Diabetes Registry.” Diabetes/metabolism research and reviews 29.5 (2013): 384-390.]

Diabetes is associated with increased mortality following acute myocardial infarction compared to the general population. Elevated glycated haemoglobin in diabetic patients is also associated with increased mortality following acute myocardial infarction, while mild elevations in HbA1c are associated with impaired glucose tolerance. In logistic regression analysis HbA1c was an independent risk factor for death. Over one-third of the fatality group had an HbA1c in the highest quartile, compared to one-fifth of the surviving group (p=0.02). Elevated HbA1c is a risk marker for short term mortality following acute myocardial, Table 14. [Chowdhury, T. A., and S. S. Lasker. “Elevated glycated haemoglobin in non-diabetic patients is associated with an increased mortality in myocardial infarction.” Postgraduate medical journal 74.874 (1998): 480-481.]

TABLE 14
Fatalities and survivors of acute myocardial
infarction divided into quartiles of HbA1c

| HbA_{1c} | <4.0 | 4.1-5.2 | 5.2-6.0 | >6.1 |
|---|---|---|---|---|
| Dead | 5 (10.8) | 10 (21.7) | 14 (30.4) | 17 (36.9) |
| Alive | 59 (28.5) | 56 (27.0) | 49 (23.7) | 43 (20.8) |

Data are n (%), χ² = 9.881, p = 0.02.

HbA1c values may be indicators of increased risk of CVD mortality in a general older population without known diabetes, Table 15. [De Vegt, F., et al. “Hyperglycaemia is associated with all-cause and cardiovascular mortality in the Hoorn population: the Hoorn Study.” Diabetologia 42.8 (1999): 926-931. Nakanishi, S., et al. “Relationship between HbA1c and mortality in a Japanese population.” Diabetologia 48.2 (2005): 230-234.]

TABLE 15
Relative risk (95% CI) of all-cause and cardiovascular mortality by categories of HbA1c

| HbA_{1c} | | | | | p for |
|---|---|---|---|---|---|
| (%)^{a}n | <5.2 (752) | 5.2-5.5 (798) | 5.6-6.4 (730) | ≥6.5 (83) | linear trend |
| all-cause mortality (n) | 41 | 55 | 72 | 17 | |
| model 1 | 1 | 1.03 (0.68-1.55) | 1.24 (0.84-1.84) | 2.23 (1.24-4.01) | 0.03 |
| model 2 | 1 | 0.94 (0.62-1.40) | 0.97 (0.65-1.45) | 1.38 (0.74-2.55) | 0.59 |
| CVD mortality (n) | 16 | 32 | 39 | 11 | |
| model 1 | 1 | 1.56 (0.85-2.84) | 1.69 (0.93-3.06) | 3.58 (1.60-8.00) | <0.01 |
| model 2 | 1 | 1.30 (0.71-2.38) | 1.09 (0.59-2.00) | 1.79 (0.77-4.16) | 0.49 |

model 1: adjusted for age and sex
model 2: additionally adjusted for hypertension, waist:hip ratio, triglycerides, LDL-cholesterol and cigarette smoking
^{a}Tertiles of HbA_{1c} were made and the highest tertile was divided into 2 subgroups by the cut-off point 6.5%

A value of 6.5% is a commonly employed cut-off point in studies exploring HbA1c levels and mortality association. The 6.5% is considered the threshold above which there is an increase risk in microvascular events and death in diabetes patients. [Nicholas, Jennifer, et al. “Recent HbA1c values and mortality risk in type 2 diabetes. Population-based case-control study.” PloS one 8.7 (2013): e68008.]

HbA1c reference ranges are standardized. WebMD cites the following ranges and associated risks of diabetes:

For people without diabetes, the normal range for the hemoglobin A1c test is between 4% and 5.6%. Hemoglobin A1c levels between 5.7% and 6.4% indicate increased risk of diabetes, and levels of 6.5% or higher indicate diabetes. The higher the hemoglobin A1c, the higher the risks of developing complications related to diabetes.

In exemplary embodiments, HbA1c values >4% (untreated) may be considered the upper limit for optimum health. This is based on an increased risk of chronic disease morbidity, pre-diabetes, and mortality. This value is substantially lower compared to the current view of health risk and HbA1c levels.

In various embodiments, HbA1c contributes to a subject's chronic disease temperature as follows:

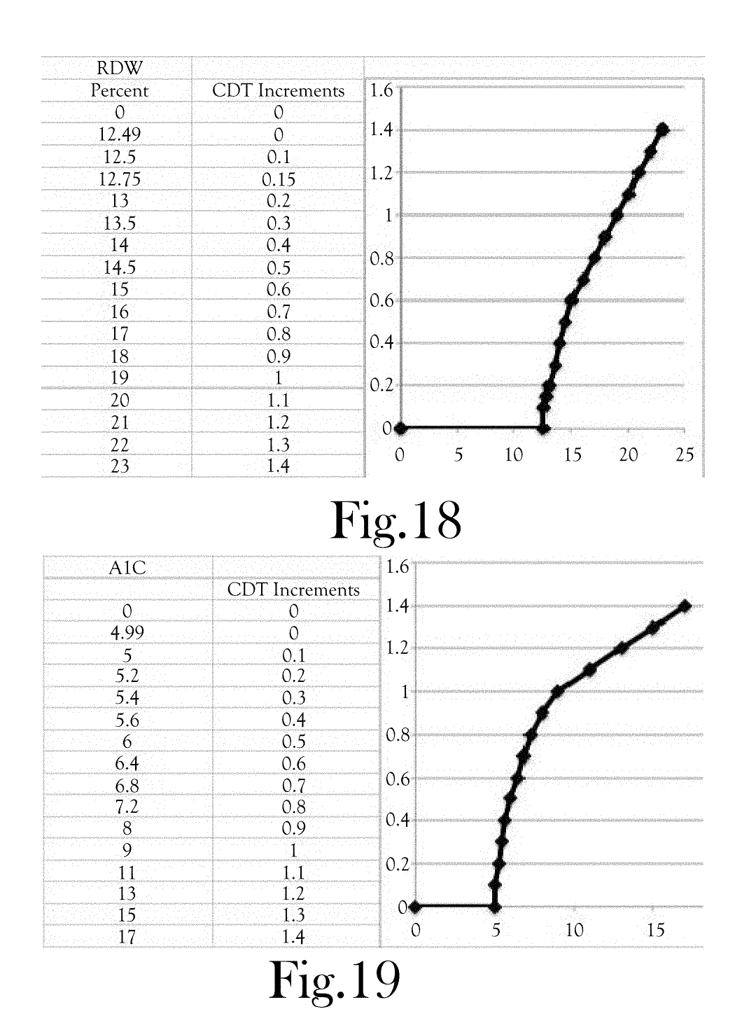

Total maximum contribution to the CDT calculation is 1.4° F. (0.78° C.). See FIG. 19.

Leptin to Adiponectin Ratio

Adiponectin: In various embodiments, adiponectin is used as a biomarker. It is a protein hormone that modulates a number of metabolic processes, including glucose regulation and fatty acid catabolism. Adiponectin is an adipocyte-specific secretory protein that circulates in serum in at least 3 forms: low molecular weight, middle molecular weight, and high molecular weight (HMW) that it is the active form of Adiponectin. Serum adiponectin level is reported to correlate well with insulin sensitivity and lipid metabolism.

Adiponectin is exclusively secreted from adipose tissue into the bloodstream and is very abundant in plasma relative to many hormones. Adiponectin is an adipocytokine released by the adipose tissue and has multiple roles in the immune system and in the metabolic syndromes such as cardiovascular disease, Type 2 diabetes, obesity and also in the neurodegenerative disorders including Alzheimer's disease. Adiponectin regulates the sensitivity of insulin, fatty acid catabolism, glucose homeostasis and anti-inflammatory system through various mechanisms. Adiponectin values are useful as a predictive biomarker for insulin resistance and as a monitoring tool in the treatment of insulin resistance related disorders and other chronic diseases of inflammation. Full-length adiponectin (f-Ad) is a 30 kDa serum protein specifically secreted by adipocytes. Adiponectin typically circulates in human blood at concentrations ranging between 5 and 12 mg/L, thus accounting for approximately 0.01% of total plasma protein. [Schondorf et al, CHn. Lab., 2005, 51: 489-494.] Adiponectin concentrations have higher median values in females (about 8.7 mg/L) than in males (about 5.5 mg/L), and may be affected by age as well. Adiponectin concentrations correlate negatively with BMI, visceral fat mass and insulin concentrations. Accordingly, adiponectin is decreased in obese subjects and in patients suffering from type 2 diabetes, macroangiopathy or other metabolic disorders. The lowest adiponectin values have been found in obese patients with both type 2 diabetes and coronary heart disease. Lower levels of adiponectin were associated with cognitive dysfunction, though it did not predict additional cognitive decline and conversion to dementia in all cases. Decreased adiponectin may be a surrogate marker of the pathological process in Alzheimer's disease, linking clinical comorbidities, inflammation and cognitive dysfunction. [Teixeira, Antonio L., et al. “Decreased levels of circulating adiponectin in mild cognitive impairment and Alzheimer's disease.” Neuromolecular medicine 15.1 (2013): 115-121.] In addition, the level of adiponectin in plasma reflects its level in CSF. The tendency to have higher adiponectin in plasma and CSF from mild cognitive impairment and Alzheimer's disease suggests that this molecule plays a critical role in the onset of AD.

A number of compounds have been shown to affect adiponectin concentrations in a subject. Pfutzner et al., Diabetes, Stoffwechsel undHerz, 2007, 16: 91-97 have shown that sulfonylurea, metformin, thiazolidinedione, metformin+sulfonylurea, metformin+thiazolidinedione, sulfonylurea+thiazolidinedione, and metformin+sulfonylurea+thiazolidinedione may have an effect on adiponectin concentrations. In placebo-controlled randomized clinical trial, fish oil moderately increased circulating adiponectin. These findings provide no evidence for harm and support possible benefits of n-3 PUFA consumption on insulin sensitivity and adipocyte function. [Wu, Jason H Y, Leah E. Cahill, and Dariush Mozaffarian. “Effect of fish oil on circulating adiponectin: a systematic review and meta-analysis of randomized controlled trials.” The Journal of Clinical Endocrinology & Metabolism 98.6 (2013): 2451-2459.]

In exemplary embodiments, a sample (such as blood) concentration of >10 mg/L indicates a very low risk for arteriosclerosis, insulin resistance and other complications; 7-10 mg/L a low risk, <7-4 mg/L a medium risk, and <4 mg/L a high risk. It is possible that a subject responding to a therapy, as shown by changes in other biomarkers, but levels of adiponectin are not changing in a significant way, since adiponectin suppression reflects the activity of the visceral adipose tissue, which may not be affected by the selected intervention.

Adiponectin reference ranges vary according to body mass index (BMI).

| Body Mass Index | Males (mcg/mL) | Females (mcg/mL |
|---|---|---|
| <25 kg/meters-squared | 4-26 | 5-37 |
| 25-30 kg/meters-squared | 4-20 | 5-28 |
| >30 kg/meters-squared | 2-20 | 4-22 |

Leptin: In various embodiments, leptin is used as a biomarker. Leptin is a 16 kDa adipose-derived protein hormone that plays a role in regulating energy intake and energy expenditure, including appetite and metabolism. The adipose tissue has been found to be an important endocrine organ in recent years. It secretes several bioactivity molecules termed adipokines regulating whole body metabolism and immune responses. Leptin is one of the important adipokines identified in 1994 [11]. It regulates the mass of adipose tissue and body weight by inhibiting food intake and stimulating energy expenditure. Many studies suggested the leptin levels were positively correlated with obesity, DM, hypertension.

Leptin also has several endocrine functions and is involved in the regulation of immune and inflammatory responses, hematopoiesis, angiogenesis and wound healing. Mutations in the leptin gene and/or its regulatory regions cause severe obesity, and morbid obesity with hypogonadism. The leptin gene has also been linked to type 2 diabetes mellitus development. Disease risk levels associated with various concentrations of leptin in human subjects is assigned as follows: Leptin Concentration (adult male) (ng/niL) Disease Risk Level >30 high; 20-30 medium; <20 low. Leptin Concentration (adult female) (ng/mL) Disease Risk Level >60 high; 40-60 medium; <40 low. [Smith, Megan B., et al. “Vitamin D excess is significantly associated with risk of atrial fibrillation.” Circulation 124.21 Supplement (2011): A14699.]

Adipose tissue-expressed adiponectin levels are inversely related to the degree of adiposity. Adiponectin concentrations correlate negatively with glucose, insulin, triacylglycerol concentrations, liver fat content and body mass index and positively with high-density lipoprotein-cholesterol levels, hepatic insulin sensitivity and insulin-stimulated glucose disposal. Adiponectin has been shown to increase insulin sensitivity and decrease plasma glucose by increasing tissue fat oxidation. The HMW is the most active form in suppressing hepatic glucose production and only HMW selectively suppressed endothelial cell apoptosis, whereas neither the low nor the middle molecular weight form had this effect. [Falahi, Ebrahim, Amir Hossein Khalkhali Rad, and Sajjad Roosta. “What is the best biomarker for metabolic syndrome diagnosis.” Diabetes & Metabolic Syndrome: Clinical Research & Reviews (2013).]

Leptin is higher in metabolic syndromes group and adiponectin is lower (<4 mg/ml) and it shows the paradoxical effect of them in metabolic syndrome. Higher leptin/adiponectin ratio is a better biomarker for metabolic syndrome diagnosis criteria than leptin and adiponectin separately.

HMW adiponectin (<2.5 mg/ml) can be the most reliable biomarker for metabolic syndrome diagnosis criteria.

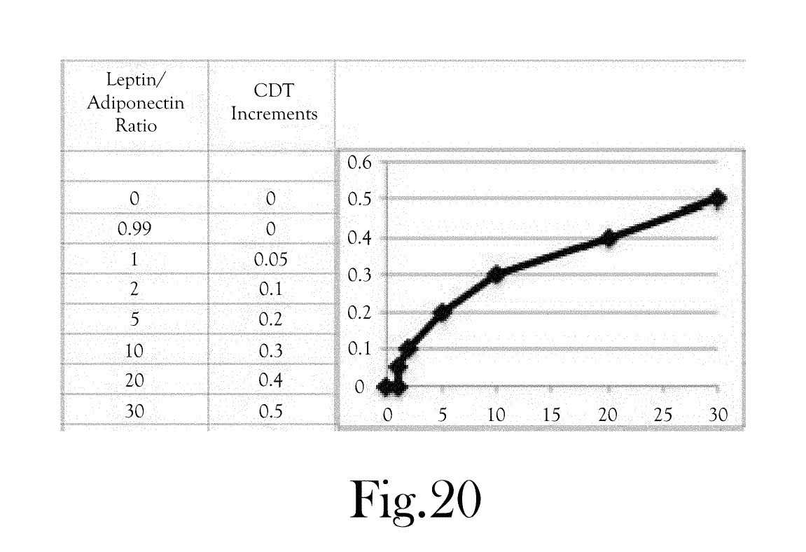

In various embodiments, the leptin/adiponectin ratio contributes to a subject's chronic disease temperature as follows: Total maximum contribution to the CDT calculation is 0.5° F. (0.28° C.). See FIG. 20.

Fibrinogen

In various embodiments, fibrinogen is used as a biomarker. Fibrinogen is a glycoprotein in vertebrates that helps in the formation of blood clots. The fibrinogen molecule is a soluble, large, and complex glycoprotein, 340 kDa plasma glycoprotein, that is converted by thrombin into fibrin during blood clot formation. It has a rod-like shape with dimensions of 9×47.5×6 nm and it shows a negative net charge at physiological pH (IP at pH 5.2). Fibrinogen is synthesized in the liver by the hepatocytes. The concentration of fibrinogen in the blood plasma is 200-400 mg/dL. It is an acute phase reactant, meaning that fibrinogen concentrations may rise sharply in any condition that causes inflammation or tissue damage. Low fibrinogen levels can also cause thrombosis due to increase in coagulation activity. Thrombosis is the formation of a blood clot inside a blood vessel. The clot blocks the normal flow of blood through the circulatory system. This can lead to heart attack and stroke.

The interaction of coagulation factors with the perivascular environment affects the development of disease in ways that extend beyond their traditional roles in the acute hemostatic cascade. Key molecular players of the coagulation cascade like tissue factor, thrombin, and fibrinogen are epidemiologically and mechanistically linked with diseases with an inflammatory component. Moreover, the identification of novel molecular mechanisms linking coagulation and inflammation has highlighted factors of the coagulation cascade as new targets for therapeutic intervention in a wide range of inflammatory human diseases. In particular, a proinflammatory role for fibrinogen has been reported in vascular wall disease, stroke, spinal cord injury, brain trauma, multiple sclerosis, Alzheimer's disease, rheumatoid arthritis, bacterial infection, colitis, lung and kidney fibrosis, Duchenne muscular dystrophy, and several types of cancer. Genetic and pharmacologic studies have unraveled pivotal roles for fibrinogen in determining the extent of local or systemic inflammation. As cellular and molecular mechanisms for fibrinogen functions in tissues are identified, the role of fibrinogen is evolving from a marker of vascular rapture to a multi-faceted signaling molecule with a wide spectrum of functions that can tip the balance between hemostasis and thrombosis, coagulation and fibrosis, protection from infection and extensive inflammation, and eventually life and death. [Davalos, Dimitrios, and Katerina Akassoglou. “Fibrinogen as a key regulator of inflammation in disease.” Seminars in immunopathology. Vol. 34. No. 1. Springer-Verlag, 2012.]

Fibrinogen values are useful as a predictive biomarker for tissue inflammation. Elevated concentrations of fibrinogen are not specific and convey a message that a subject with elevated fibrinogen is at risk of one or more of a myriad of chronic afflictions. While fibrinogen levels are elevated, a subject's risk of developing a blood clot may be increased and, over time, to an increased risk for developing cardiovascular disease. Elevated levels may be seen with acute infections, Cancer, coronary heart disease, myocardial infarction, stroke, inflammatory disorders (like rheumatoid arthritis and glomerulonephritis, a form of kidney disease), trauma, cigarette smoking, pregnancy, peripheral artery disease, and a general increase in all-cause mortality in patients with peripheral arterial disease. Increased levels of fibrinogen in the blood is an independent risk factor for mortality in patients with peripheral arterial disease. When left untreated, peripheral arterial disease increases the risk of heart attack, stroke, and death. Death from all causes increased with elevated fibrinogen levels: 80% of patients with a fibrinogen level above 340 mg/dL, and who had peripheral arterial disease, survived for less than three years. [Cheuk B L, Cheung G C, Lau S S, Cheng S W. Plasma fibrinogen level: an independent risk factor for long-term survival in Chinese patients with peripheral artery disease. World J Surg. 2005 October; 29 (10):1263-7.] Fibrinogen levels have been shown by a number of research teams to rise about 25 mg/dl per decade of age. [Yarnell, J. W., et al. “Fibrinogen, viscosity, and white blood cell count are major risk factors for ischemic heart disease. The Caerphilly and Speedwell collaborative heart disease studies.” Circulation 83.3 (1991): 836-844.]

During the tenth biennial examination of the Framingham Study, 1315 participants who were free of cardiovascular disease had fibrinogen levels measured. During the ensuing 12 years, cardiovascular disease developed in 165 men and 147 women. For both sexes, the risk of cardiovascular disease was correlated positively to antecedent fibrinogen values higher than the 1.3 to 7.0 g/L (126 to 696 mg/dL) range. The magnitude of the risk diminished with advancing age in women but not in men. Risk for coronary heart disease also was significantly related to fibrinogen level. Here, the magnitude of risk displayed diminishing impact with age, again only in women. Risk of stroke increased progressively with fibrinogen level in men but not in women. The impact of fibrinogen value, considered as a separate variable, on cardiovascular disease was comparable with the major risk factors, such as blood pressure, hematocrit, adiposity, cigarette smoking, and diabetes. Fibrinogen values were also significantly related to these risk factors. Taking all these into account in a multivariate analysis, fibrinogen level was still significantly related to the incidence of cardiovascular disease in men and marginally significant in women. For coronary heart disease, the fibrinogen level was significant for both men and women. Elevated fibrinogen level is a predictor of cardiovascular disease. [Kannel, William B., et al. “Fibrinogen and risk of cardiovascular disease: the Framingham Study.” Jama 258.9 (1987): 1183-1186.]

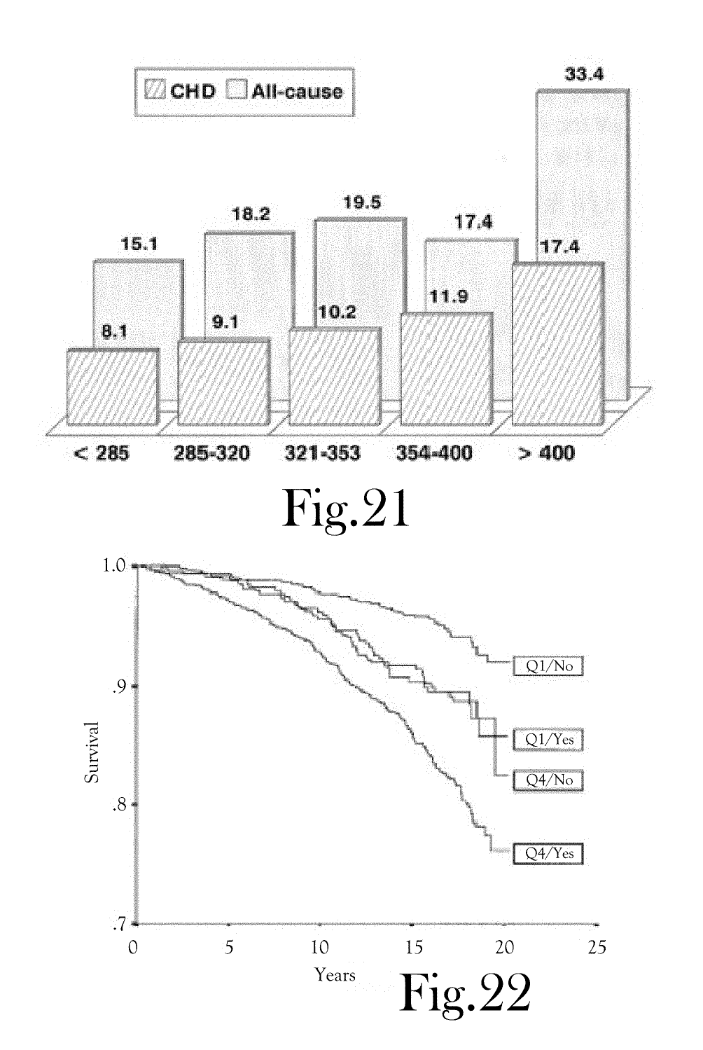

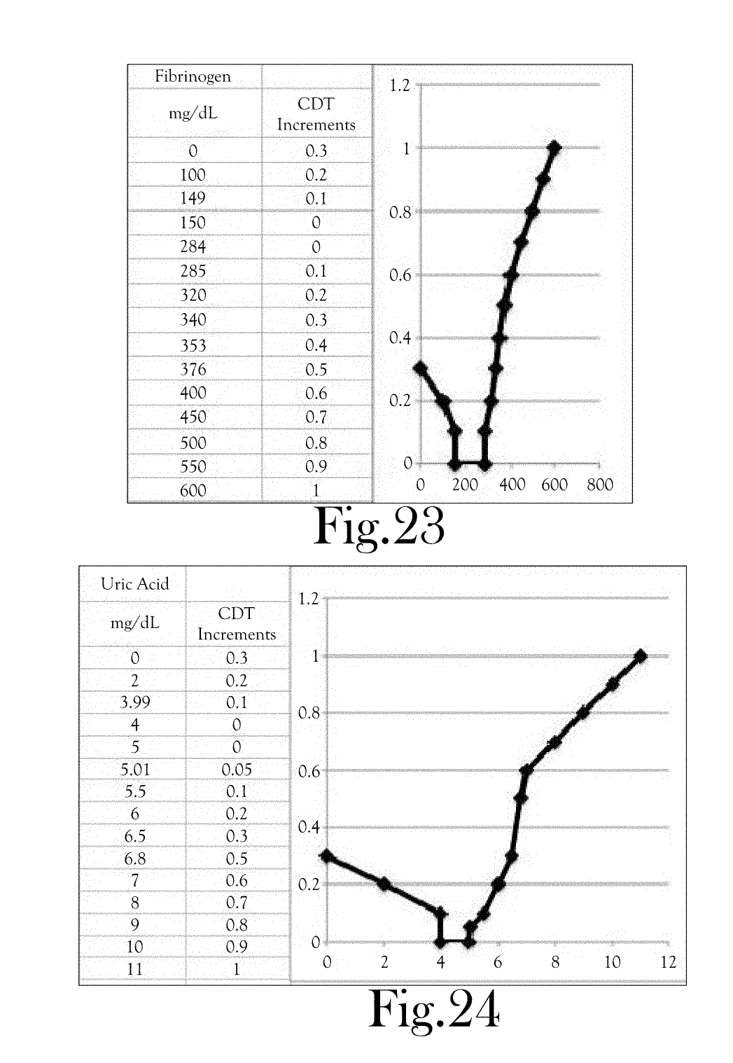

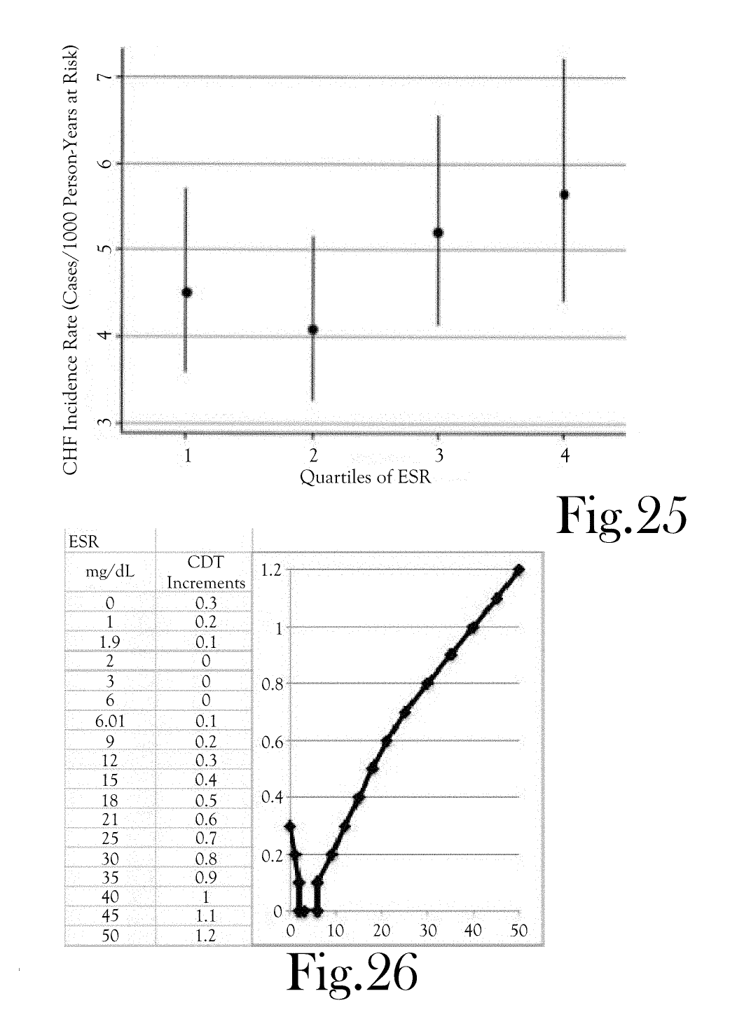

The role of fibrinogen as a primary cardiovascular risk factor is well established and has been demonstrated by a number of prospective epidemiological studies of healthy individuals. In a meta-analysis of six prospective studies, the odds of sustaining a cardiovascular event in healthy persons with a fibrinogen level in the highest tertile were 2.3 times as high as in those with fibrinogen levels in the lowest tertile (low, <308.7 mg/dl; medium 308.7-367.9 mg/dl; high ≥368.0 mg/dl). In subjects with cardiovascular disease, an increase of 100 mg/dL of fibrinogen in patients with stable intermittent claudication predicted a nearly twofold increase in the probability of death within the next 6 years. Another study of 1716 men 6 months after an index MI reported a trend of increasing odds of ischemic events with increasing fibrinogen levels during 2.5 years of follow-up. An increase in fibrinogen of 75 mg/dl is considered to be about 1 standard deviation. FIG. 6 shows that all-cause mortality increased from 15.1 in the bottom quintile to 33.4 in the highest quintile (test for linear trend: P<0.0001). The rate of mortality attributed to CHD ranged between 8.1 in the lowest quintile and 17.4 in the highest one (test for linear trend: P=0.0001). [Benderly, Michal, et al. “Fibrinogen is a predictor of mortality in coronary heart disease patients.” Arteriosclerosis, thrombosis, and vascular biology 16.3 (1996): 351-356.] FIG. 21 shows the age-adjusted mortality rates per 1000 person-years by fibrinogen quintiles.

In a study on cardiovascular diseases and death, risk as a function of fibrinogen quartiles and changes in relation to levels of other inflammation-sensitive plasma proteins was evaluated. The study included incidence of cardiac events and death in men in relation to fibrinogen levels alone and in combination with other proteins. The study was based on 6075 men, who were, on average, 46 years old at the time of the screening examination, which included the quantitative assessment of plasma levels of fibrinogen, orosomucoid, α1-antitrypsin, haptoglobin, and ceruloplasmin. The concentration of each protein was divided into quartiles for each. For fibrinogen the quartiles were assigned as: Fibrinogen, g/L 2.56+−0.31 3.20+−0.15 3.68+−0.15 4.52+−0.55. This classification made it possible to identify 4 groups, i.e. men in the first fibrinogen quartile and at the same time either not belonging to the fourth quartile of any of the other proteins (Q1/No group) or also belonging to the fourth quartile of ≥1 of the additional proteins (Q1/Yes group) and corresponding groups in the fourth fibrinogen quartile (Q4/No and Q4/Yes groups). During the follow-up, which occurred at an average of 16 years, 439 (7.2%) men experienced a cardiac event, and 653 (10.7%) died; 278 of these men died of cardiovascular diseases, with 206 deaths attributed to ischemic heart disease. From the lowest to the highest quartile, there was for each protein a stepwise increase in the incidence of cardiac events and mortality. All-cause mortality and cardiovascular mortality were significantly higher in the Q4/Yes group compared with the Q4/No group, but they were similar in the Q4/No and Q1/Yes groups. The incidence of cardiac events was significantly higher in the Q1/Yes and Q4/Yes groups compared with the Q1/No and Q4/No groups, respectively. The increased cardiovascular mortality and cardiac event rates remained after adjustment for several confounders when the Q4/Yes and Q4/No groups were compared. The results suggest that the incidence of cardiac events and death due to cardiovascular diseases in middle-aged men predicted by plasma levels of fibrinogen is modified by other inflammation-sensitive proteins., [Lind, P., et al. “Influence of Plasma Fibrinogen Levels on the Incidence of Myocardial Infarction and Death Is Modified by Other Inflammation-Sensitive Proteins A Long-Term Cohort Study.” Arteriosclerosis, thrombosis, and vascular biology 21.3 (2001): 452-458.]

As shown in FIG. 22, on average, 16-years all-cause mortality rates in middle-aged men in relation to plasma levels of inflammation-sensitive proteins, ie, lowest (Q1) and highest (Q4) fibrinogen quartile with (Yes) and without (No)>=1 of the other proteins, i.e. orosomucoid, alpha1-anttrypsin, haptoglobin, and ceruloplasmin, in top quartile baseline.

Decreased fibrinogen levels (<100 mg/dL) are associated with the following: Afibrinogenemia, Hypofibrinogenemia, end-stage liver disease, and severe malnutrition. [Fibrinogen. Lab Tests Online: Welcome!. Available at http://labtestsonline.org/understanding. Accessed: Aug. 13, 2012.]

An example of fibrinogen reference values are as follows:

Fibrinogen antigen: 149-353 mg/dL; Fibrinogen: 150-400 mg/dL; Fibrinogen antigen/functional ratio: 0.59-1.23

Fibrinogen levels can be measured in venous blood. Normal levels are about 1.5-3 g/L, depending on the method used. In typical circumstances, fibrinogen is measured in citrated plasma samples in the laboratory, however the analysis of whole-blood samples by use of thromboelastometry (platelet function is inhibited with cytochalasin D) is also possible. Higher levels are, amongst others, associated with cardiovascular disease (>3.43 g/L). It may be elevated in any form of inflammation, as it is an acute-phase protein; for example, it is especially apparent in human gingival tissue during the initial phase of periodontal disease. Fibrinogen levels increase in pregnancy to an average of 4.5 g/l, compared to an average of 3 g/l in non-pregnant people.

In exemplary embodiments, fibrinogen values >100 mg/dl and <285 mg/dl may be considered the range for good health. This is based on the risk of cardiovascular diseases and all-cause mortality. Risk increases with increasing fibrinogen value with a change of 75 mg/dl being considered one standard deviation. In subjects with cardiovascular disease, an increase of 100 mg/dL of fibrinogen in patients with stable intermittent claudication predicted a nearly twofold increase in the probability of death within the next 6 years.

In various embodiments, fibrinogen contributes to a subject's chronic disease temperature as follows: Total maximum contribution to the CDT calculation is 1.0° F. (0.56° C.). See FIG. 23.

Uric Acid

In various embodiments, uric acid is used as a biomarker. Uric acid is the final product of purine metabolism in humans. Purines are components of nucleosides, the building blocks of DNA and RNA. Purine nucleosides (adenosine and guanine) are used in the creation of other metabolically important factors as well, such as adensosine triphosphate (ATP; the energy-carrying molecule), S-adeneosylmethione (SAMe; the methyl donor), and nicotine adenine dinucleotide (NADH; an important cofactor in energy production and antioxidation). Given the importance of purine-containing molecules for survival, vertebrates, including humans, have developed robust systems for synthesizing sufficient purine nucleosides for their metabolism using readily available materials (such as glucose, glycine, and glutamine), as well as recycling purine nucleosides from throughout the body or from the diet.

Uric acid passes through the liver, and enters the bloodstream. If there is more uric acid than the kidneys can get rid of, a condition called hyperuricemia develops. Uric acid crystals may form when the blood uric acid level rises above 7 mg/dL. Problems, such as kidney stones, and gout may occur. Most of it is excreted in the urine, or passes through your intestines to regulate “normal” levels.

The levels of uric acid in the blood depend on two factors. The first is the rate of uric acid synthesis in the liver. Since uric acid results from purine degradation, its levels are influenced by both the amount of purines synthesized in the body, as well as the amounts of purines absorbed from the diet. [Richette P, Bardin T. Gout. Lancet 2010; 375:318-28.] The second determinant of blood uric acid levels is the rate of uric acid excretion from the kidneys. Excretion has the greatest effect on blood uric acid levels, with about 90% of hyperuricemia cases attributed to impaired renal excretion. [Choi H K, Mount D B, Reginato A M, American College of Physicians, American Physiological Society. Pathogenesis of gout. Ann Intern Med. 2005; 143(7):499-516.] Impaired excretion is most often due to abnormalities in the kidney urate transporter (called URAT1) or organic ion transporter (OAT), both of which control the movement of uric acid out of proximal kidney tubules and into urine. [Enomoto, A. et al. Molecular identification of a renal urate anion exchanger that regulates blood urate levels. Nature 2002; 417, 447-452.] Only about 10% of the uric acid that enters a normal human kidney is excreted from the body. Uric acid is recycled to provide antioxidant properties and is responsible for the neutralization of over 50% of the free radicals in the blood stream. [Glantzounis G K, Tsimoyiannis E C, Kappas A M, et al. Uric acid and oxidative stress. Curr Pharm Des. 2005; 11(32):4145-51.]

Humans and primates are one of the few mammals that cannot produce their own vitamin C, and may have evolved the ability to preserve uric acid to compensate for this. For example, blood uric acid levels in humans are normally about 6 times that of vitamin C, and about ten times the levels in other mammals. Like vitamin C, uric acid has a principle role in protecting high-oxygen tissues (like the brain) from damage, and low blood uric acid levels have been associated with the progression or increased risk of several neurological disorders, including Amyotrophic Lateral Sclerosis, Multiple sclerosis, and Huntington's, Parkinson's, and Alzheimer's diseases. [Kim T S, Pae C U, Yoon S J, Jang W Y, Lee N J, Kim J J, et al. Decreased plasma antioxidants in patients with Alzheimer's disease. Int J Geriatr Psychiatry 2006; 21:344-8.]

Uric acid is a metabolic “waste product” with poor solubility in body fluids, yet its potential role as a primary antioxidant in body fluids suggests that it should be kept at sufficient levels in the blood. These diametric properties of uric acid define a range for normal blood uric acid levels. Commonly, the upper limit of this range is taken as 8.6 mg/dl in men and 7.1 mg/dl in women. Uric acid levels above this limit are considered as hyperuricemia. Hyperuricemia is a primary risk factor for the development of gout, although it is likely that many hyperuricemic individuals will not develop symptoms. While the risk of a gout attack increases with blood uric acid, the annual occurrence of inflammatory gout is fairly low; persons with blood uric acid levels between 7 and 8.9 mg/dL have a 0.5-3% change of developing the disease, which rises to 4.5% at levels over 9 mg/dL. [Campion E W, Glynn R J, DeLabry L O. Asymptomatic hyper-uricemia. Risk and consequences in the Normative Aging Study. Am J Med 1987; 82:421-6.]

Altered serum uric acid concentrations, both above and below normal levels, have been linked to a number of disease states. An abnormally high uric acid level has been correlated with gout, hypertension, cardiovascular disease, and renal disease, whereas a reduced uric acid concentration has been linked to multiple sclerosis, Parkinson's disease, Alzheimer's disease, and optic neuritis.

Elevated blood levels of uric acid have also been associated diseases other than gout. Hyperuricemia and gout are both risk factors for kidney or bladder stones (urolithiasis). Both conditions increase the risk of forming not only uric acid stones, but also the more common calcium oxalate stones. The presence of calcium oxalate stones is 10-30 times higher in gout patients than those without gout. Hyperuricemia is a risk factor for cardiovascular diseases in high risk groups, and has been associated with small increases in the risk of coronary events, heart failure, and stroke. It is often seen in patients with hypertension. A comprehensive review of 18 observational studies revealed that for each 1 mg/dl increase in blood uric acid, the risk of hypertension increased by 13%. [Grayson P C, Kim S Y, LaValley M, Choi H K. Hyperuricemia and incident hypertension: a systematic review and meta-analysis. Arthritis Care Res. 2011; 63(1):102-110.] Data from the Multiple Risk Factor Intervention Trial (MRFIT) showed that hyperuricemia was associated with increased risk of type 2 diabetes, and that male patients with gout had a 41% increased risk for the disease. [Choi H K, De Vera M A, Krishnan E. Gout and the risk of type 2 diabetes among men with a high cardiovascular risk profile. Rheumatology (Oxford). 2008a; 47(10):1567-1570.]

There is a strong relationship between serum uric acid and mortality. In a study of 1423 middle-aged Finnish men, an increase in all-cause mortality risk between the lowest and highest tertiles (3.03-5.08 mg/dL) and highest (5.89-9.58 mg/dL) tertiles of baseline SUA concentrations (RR 1.82-1.12-2.97, p=0.02) and cardiovascular mortality risk was greater in those with the highest SUA concentrations (RR 3.73, 1.42-9.83, p=0.01). [Barron, Evelyn, et al. “Blood-borne biomarkers of mortality risk: systematic review of cohort studies.” PloS one 10.6 (2015): e0127550.] Wu et al reported a significant association between SUA and all-cause mortality in male participants in NHANES III with low CV risk (HR 1.15, 1.04-1.27, p=0.007). [Wu C K, Chang M H, Lin J W, Caffrey J L, Lin Y S. Renal-related biomarkers and long-term mortality in the US subjects with different coronary risks. Atherosclerosis. 2011; 216:226-36.] In a large cohort of 28,613 Austrian women, Strasak et al reported greater risk of cardiovascular mortality in those in the highest versus the lowest quartiles of serum uric acid (HR 1.52, 1.37-1.70; p<0.0001). Uric acid in the highest quartile (≥5.41 mg/dL) was significantly associated with mortality from total CVD (p<0.0001), showing a clear dose-response relationship; the adjusted hazard ratio (95% CI) in comparison to the lowest serum uric quartile was 1.35 (1.20-1.52). In subgroup analyses serum uric was independently predictive for deaths from acute and subacute (p<0.0001) and chronic forms (p=0.035) of CHD, yielding adjusted hazard ratios for the highest versus lowest serum uric acid quartile of 1.58 (1.19-2.10) and 1.25 (1.01-1.56), respectively. Serum uric acid was further significantly related to fatal CHF (p<0.0001) and stroke (p=0.018); the adjusted hazard ratios for the highest versus lowest serum uric acid quartile were 1.50 (1.04-2.17) and 1.37 (1.09-1.74), respectively. These findings, demonstrate that serum uric acid is an independent predictor for all major forms of death from CVD including acute, subacute and chronic forms of CHD, CHF and stroke in elderly, post-menopausal women. [Strasak A M, Kelleher C C, Brant L J, Rapp K, Ruttmann E, Concin H, et al. Serum uric acid is an independent predictor for all major forms of cardiovascular death in 28,613 elderly women: A prospective 21-year follow-up study. International Journal of Cardiology. 2008]

The relationships of serum uric acid to mortality from all causes, the cardiovascular diseases, and cancer were evaluated in 6797 white women age 35-64 years followed for an average of 11.5 years in the Chicago Heart Association Detection Project in Industry (CHA). Serum uric acid levels at baseline were strongly and significantly associated with all causes mortality in this cohort, with control for multiple risk factors and with exclusion of hypertensives on treatment. [Levine, William, et al. “Serum uric acid and 11.5-year mortality of middle-aged women: findings of the Chicago Heart Association Detection Project in Industry.” Journal of clinical epidemiology 42.3 (1989): 257-267.]

Data from 1,044 men who are members of the World Health Organization Monitoring Trends and Determinants in Cardiovascular Diseases (MONICA) Augsburg cohort were evaluated. The men, 45-64 years of age in 1984-1985, were followed through 1992. There were 90 deaths, 44 of which were related to cardiovascular disease; 60 men developed incident nonfatal or fatal myocardial infarction. Uric acid levels >=373 [mu]mol/liter (fourth quartile) vs <=319 [mu]mol/liter (first and second quartile) independently predicted all-cause mortality [hazard rate ratio=2.8; 95% confidence interval (CI)=1.6-5.0] after adjustment for alcohol, total cholesterol/high-density lipoprotein cholesterol ratio, hypertension, use of diuretic drugs, smoking, body mass index, and education. The adjusted risk of cardiovascular disease mortality was 2.2 (95% CI=1.0-4.8), and that of myocardial infarction was 1.7 (95% CI=0.8-3.3). [Liese, Angela D., et al. “Association of Serum Uric Acid with All-Cause and Cardiovascular Disease Mortality and Incident Myocardial Infarction in the MONICA Augsburg Cohort.” Epidemiology 10.4 (1999): 391-397.]

Serum uric acid levels that are below normal concentrations have also been linked to a variety of disease states, including multiple sclerosis, optic neuritis, Parkinson's disease, and Alzheimer's disease. In these inflammatory diseases, a decreased uric acid concentration may not be able to prevent the toxicity by reactive oxygen and nitrogen species that form as a result of the inflammation. Peroxynitrite, in particular, is believed to have a significant negative impact on cellular function and survival. Uric acid is chronically low in neurodegenerative diseases including Parkinson's, ALS, and Alzheimer's disease. The ALS patients' mean±SD uric acid level was significantly lower (4.78±1.3 mg/dl) than that of the controls (5.76±1.26 mg/dl) (p<0.0001). Uric acid is a natural antioxidant, accounting for up to 60% of the free radical scavenging activity in human blood. Uric acid can scavenge superoxide, the hydroxyl radical, and singlet oxygen. [Ames B N, Cathcart R, Schwiers E, and Hochstein P (1981) Uric acid provides an antioxidant defense in humans against oxidant- and radical-caused aging and cancer: a hypothesis. Proc Natl Acad Sci USA 78: 6858-6862.] Uric acid may assist in the removal of superoxide by preventing against the degradation of superoxide dismutase, the enzyme that is responsible for clearing superoxide from the cell. Removal of superoxide helps to prevent its reaction with NO, blocking the formation of peroxynitrite. Thus, a reduced uric acid concentration may decrease the ability of the body to prevent peroxynitrite and other free radicals from acting on cellular components and damaging the cell. [Kutzing, Melinda K., and Bonnie L. Firestein. “Altered uric acid levels and disease states.” Journal of Pharmacology and Experimental Therapeutics 324.1 (2008): 1-7.]

Uric Acid Reference Ranges:

| | | |
|---|---|---|
| Men: | 3.4-7.0 milligrams | 202-416 micromoles |
| | per deciliter (mg/dL) | per liter (mcmol/L) |
| Women: | 2.4-6.0 mg/dL | 143-357 mcmol/L |
| Children: | 2.0-5.5 mg/dL | 119-327 mcmol/L |

In various embodiments, uric acid contributes to a subject's chronic disease temperature as follows: Total maximum contribution to the CDT calculation is 1.0° F. (0.56° C.). See FIG. 23.

Erythrocyte Sedimentation Rate (SED Rate, ESR)

In various embodiments, the erythrocyte sedimentation rate (ESR or sed rate) is used as a biomarker of systemic illness. The test involves placing anticoagulated whole blood into an upright test tube and monitoring the rate at which red blood cells (RBC) fall over time. Negative charges keep RBC from sticking together. If this charge is neutralized, RBC stack into chains, or rouleaux, and fall more rapidly. ESR can be measured with a variety of tests: Westergren and modified Westergren; Wintrobe; micro-ESR. The Westergren is the most commonly used method of performing the ESR. Technical factors, such as temperature, time from specimen collection, tube orientation and vibration, can affect the results. RBC size, shape and concentration impact the ESR. Plasma characteristics are also important determinants of the ESR. Other factors that can change ESR include age, sex, race, medications and disease states, such as obesity, hypofibrinogenaemia and congestive heart failure. Other acute-phase reactants besides the ESR include C-reactive protein, fibrinogen, complement, ferritin, plasma viscosity, serum amyloid A and albumin. When clinical suspicion for infection or inflammation is low, a normal ESR can reassure that there is no active disease. The slow rise (48 h) and fall of the ESR relative to other acute-phase reactants may make it superior for monitoring inflammation in more chronic conditions. In conjunction with physical findings and other laboratory values, the ESR value can be used to screen for disease or disease complications, aid in disease diagnosis or assess disease activity or response to therapy. Results from a sed rate test are reported in the distance in millimeters (mm) red blood cells have descended in one hour. The normal range is 0-22 mm/hr for men and 0-29 mm/hr for women.

An increased ESR rate may be due to: anemia, cancers such as lymphoma or multiple myeloma, kidney disease, pregnancy, thyroid disease, autoimmune disorders, Lupus, rheumatoid arthritis, systemic infection, and tuberculosis.

Inflammation, as measured by the erythrocyte sedimentation rate, is an independent predictor for the development of heart failure. This finding is based on three decades of follow-up in a population-based sample of middle-aged men. The findings indicate that inflammation occurs early in the process leading to heart failure and that ESR may be a diagnostic for this process in subjects. The hazard ratio 1.46 for highest quartile vs. the lowest, 95% confidence interval 1.04 to 2.06, FIG. 10. [Ingelsson, Erik, et al. “Inflammation, as measured by the erythrocyte sedimentation rate, is an independent predictor for the development of heart failure.” Journal of the American College of Cardiology 45.11 (2005): 1802-1806.] FIG. 10 shows the incidence rates of congestive heart failure (CHF) by quartiles (quartile 1, ESR=1 to 3 mm/h; quartile 2, 4 to 6 mm/h; quartile 3, 7 to 10 mm/h; quartile 4, 11 to 83 mm/h) of ESR. Lines indicate 95% confidence intervals.

Although the ESR varies among elderly patients, it has a positive correlation with several CHD risk factors, including age, sex, smoking, systolic blood pressure, total cholesterol levels, heart rate, body mass index, diabetes, alcohol consumption, and fibrinogen, hemoglobin, and albumin levels. After multivariate adjustment, the ESR is an independent and strong short- and long-term predictor of CHD death. In young subjects, a moderate but persistent elevation in the ESR has been associated with an increased risk of incident MI. Other conditions associated with a persistently elevated ESR include chronic infectious states, renal failure, rheumatoid arthritis, and chronic bronchitis. In the Stockholm Prospective Study, there was a positive and independent relationship between the ESR and fatal MI in asymptomatic men and women, but in NHANES I, the ESR was a risk factor for fatal MI only in men. In the Reykjavik Study, the ESR was an independent long-term predictor of CHD and death due to stroke in both men and women. Another study found that the ESR was related to the extent of coronary atherosclerosis on angiography and was a predictor of cardiac death in men with ischemic heart disease. A meta-analysis of 4 population-based studies showed that an ESR in the top third tertile yielded a risk ratio of 1.33 (95% CI, 1.15-1.54), compared with an ESR in the bottom tertile. [Madjid, Mohammad, and Omid Fatemi. “Components of the complete blood count as risk predictors for coronary heart disease: in-depth review and update.” Texas Heart Institute Journal 40.1 (2013): 17.]

The erythrocyte sedimentation rate appears, in absence of confounding conditions, to be a strong short- and long-term predictor of coronary heart disease mortality in apparently healthy, middle-aged men, Table 16. Since the erythrocyte sedimentation rate also carries strong prognostic information in men with known or suspected coronary heart disease and, since an increasing erythrocyte sedimentation rate was associated with a particularly steep gradient in the percentages of men dying from coronary heart disease without prior myocardial infarction, it is hypothesized that a high erythrocyte sedimentation rate may be an indicator of aggressive, malignant forms of coronary heart disease, conceivably by being a marker of activated humoral immune mechanisms in widespread atheromatous tissues, Table 17. [Erikssen, G., et al. “Erythrocyte sedimentation rate: a possible marker of atherosclerosis and a strong predictor of coronary heart disease mortality.” European heart journal 21.19 (2000): 1614-1620.]

TABLE 16
Total mortality and mortality from various causes after 23 years, associated
with different levels of ESR determined at Survey 1 in 1972-1975.

| ESR | | | | | | | | | | | | |
|---|---|---|---|---|---|---|---|---|---|---|---|---|
| (mm · h⁻¹) | n | SMR* | Total | % | CVD | % | CHD | % | Cancer | % | Non-CVD† | % |
| 0-4 | 805 | 0.72 | 210 | 26.1 | 104 | 12.9 | 78 | 9.7 | 66 | 8.2 | 106 | 13.2 |
| 5-9 | 745 | 0.66 | 197 | 26.4 | 103 | 13.8 | 88 | 11.8 | 56 | 7.5 | 94 | 12.6 |
| 10-14 | 256 | 0.73 | 77 | 30.1 | 35 | 13.7 | 29 | 11.3 | 25 | 9.8 | 42 | 16.4 |
| 15-29 | 172 | 1.09 | 73 | 42.4 | 43 | 25.0 | 39 | 22.7 | 14 | 8.1 | 30 | 17.4 |
| ≥30 | 36 | 1.54 | 22 | 61.1 | 12 | 33.3 | 9 | 25.0 | 6 | 16.7 | 10 | 27.8 |
| All | 2014 | 0.73 | 579 | 28.7 | 297 | 14.7 | 243 | 12.1 | 167 | 8.3 | 282 | 14.0 |

SMR = standard mortality ratio (reference: Norwegian male population, 1990).
CVD = cardiovascular disease; CHD = coronary heart disease
†Non-CVD mortality = mortality from cancer + mortality from other non-CVD causes.

TABLE 17
Relationship between ESR and coronary heart disease mortality among 403 men having
developed angina pectoris and/or a positive exercise ECG test at Survey 2.

| | Angina pectoris or | | Angina | | Positive exercise ECG test, | |
|---|---|---|---|---|---|---|
| | positive exercise ECG test | | pectoris | | not angina pectoris | |
| | n | % Dead | n | % Dead | n | Dead |
| ESR 0-4 | 26/159 | 16.4 | 10/47 | 21.2 | 16/112 | 14.3 |
| ESR 5-9 | 19/116 | 16.4 | 7/34 | 20.6 | 12/82 | 14.6 |
| ESR 10-14 | 9/56 | 16.1 | 4/17 | 23.5 | 5/39 | 16.1 |
| ESR 15-29 | 17/60 | 28.3 | 7/18 | 38.9 | 10/42 | 23.8 |
| ESR ≥30 | 6/12 | 50.0 | 3/5 | 60.0 | 3/7 | 42.9 |
| Mean | 77/403 | 19.1 | 31/121 | 25.6 | 46/282 | 16.3 |

*16 years of follow-up.

In a study of biomarkers of frailty and mortality, sedimentation rate changes (per standard deviation) were shown to be equally or more predictive of future mortality compared to all other biomarkers studied with an unadjusted hazard ratio for mortality per standard deviation increase in biomarker of 1.33. [Baylis, D., et al. “Immune-endocrine biomarkers as predictors of frailty and mortality: a 10-year longitudinal study in community-dwelling older people.” Age 35.3 (2013): 963-971.] In a study of inflammation and mortality middle-aged men who had an ESR >6 mm/h (median), the adjusted risk of cardiovascular mortality was 3.05-fold (95% CI 1.49-6.23) in the highest quartile of hematocrit compared to the lowest. This association was not observed among the men with ESR <6 mm/h. [Ingelsson, Erik, et al. “Inflammation, as measured by the erythrocyte sedimentation rate, is an independent predictor for the development of heart failure.” Journal of the American College of Cardiology 45.11 (2005): 1802-1806.]

In a study of 401 subjects (median age 75; range 65-99, 155 male, 246 female; median ESR 80 mm/h, range 50-148), 48% had a persistently raised ESR (two values >50 mm/h separated by at least 14 days; group 1); 39% had a single ESR measurement only (group 2), and 13% had a transiently raised ESR (group 3). The commonest diagnosis in group 1 patients was rheumatologic disease (51.8%), followed by infection (31.9%) and non-hematological malignancy (11%). Infection was the commonest diagnosis in groups 2 (47.4%) and 3 (43.7%), followed by non-hematological malignancy (19.9%) in group 2 and rheumatologic disease (20.4%) in group 3. In only 1 in 20 cases was no diagnosis apparent at 1 year. The standardized mortality ratio of the combined groups 1 and 2 (482; CI: 421-544) was strikingly raised, and even more so if patients with rheumatoid arthritis were excluded (542; CI 458-625). A gradient of mortality against the level of the ESR was observed. Even the lowest ESR levels (50-69 mm/h) was associated with increase of mortality between 3- and 4-fold. An ESR above 50 mm/h implies significant disease in nearly all cases and an increased mortality. [Stevens, Denise, Raymond Tallis, and Sally Hollis. “Persistent grossly elevated erythrocyte sedimentation rate in elderly people: one year follow-up of morbidity and mortality.” Gerontology 41.4 (1995): 220-226.]

In exemplary embodiments, ESR values of 3-6 mm/h or less may be considered optimal for good health.

In various embodiments, ESR contributes to a subject's chronic disease temperature as follows:

Total maximum contribution to the CDT calculation is 1.2° F. (0.67° C.). See FIG. 26.

TNF-alpha

In various embodiments, tumor necrosis factor alpha (TNF) is used as a biomarker. TNF was discovered more than a century ago as endotoxin-induced glycoprotein, which causes haemorrhagic necrosis of sarcomas. TNF is a cell signaling protein (cytokine) involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. [Gahring L C, Carlson N G, Kulmar R A, Rogers S W. “Neuronal expression of tumor necrosis factor alpha in the murine brain.” Neuroimmunomodulation. 1996 September-October; 3(5):289-303.] The primary role of TNF is in the regulation of immune cells. TNF, being an endogenous pyrogen, is able to induce fever, apoptotic cell death, cachexia, inflammation and to inhibit tumorigenesis and viral replication and respond to sepsis via IL1 & IL6 producing cells. TNF now has diverse and critical roles to play in the pathogenic progression of a number of chronic inflammatory disorders, including Rheumatoid arthritis, Crohn's disease, psoriasis, Alzheimer's disease, ischemic stroke, Parkinson's, amyotrophic lateral sclerosis and multiple sclerosis. Dysregulation of TNF production has been implicated in a variety of human diseases including Alzheimer's disease, [Swardfager W, Lanctŏt K, Rothenburg L, Wong A, Cappell J, Herrmann N (2010). “A meta-analysis of cytokines in Alzheimer's disease”. Biol Psychiatry 68 (10): 930-941] cancer, [Locksley R M, Killeen N, Lenardo M J (2001). “The TNF and TNF receptor superfamilies: integrating mammalian biology”. Cell 104 (4): 487-501.] major depression [Dowlati Y, Herrmann N, Swardfager W, Liu H, Sham L, Reim E K, Lanctŏt K L (2010). “A meta-analysis of cytokines in major depression”. Biol Psychiatry 67 (5): 446-457] and inflammatory bowel disease (IBD). [Brynskov J, Foegh P, Pedersen G, Ellervik C, Kirkegaard T, Bingham A, Saermark T (2002). “Tumour necrosis factor alpha converting enzyme (TACE) activity in the colonic mucosa of patients with inflammatory bowel disease”. Gut 51 (1): 37-43.]

TNF-alpha has been proposed to be a useful marker for clinical diagnosis of inflammation at an early stage. The serum TNF-alpha levels measured by a highly sensitive enzyme-linked immunosorbent assay (ELISA) kit were increased significantly in metabolic syndrome subjects compared with healthy individuals. High levels of TNF-alpha were found in the cerebrospinal fluid of 53 percent of the patients with chronic progressive multiple sclerosis and in none of those with stable multiple sclerosis (P less than 0.001). TNF-alpha was detected in the cerebrospinal fluid of 7 percent of the controls (P less than 0.01) with other neurologic disease. In patients with chronic progressive multiple sclerosis, mean TNF-alpha levels were significantly higher in the cerebrospinal fluid than in corresponding serum samples (52.41 vs. 11.88 U per milliliter; range, 2 to 178 vs. 2 to 39; P less than 0.001). In these patients, cerebrospinal fluid levels of TNF-alpha correlated with the degree of disability (r=0.834, P less than 0.001) and the rate of neurologic deterioration (r=0.741, P less than 0.001) before the start of the study. Cerebrospinal fluid levels also correlated with the increase in neurologic disability after 24 months of observation (r=0.873, P less than 0.001). [Sharief, Mohammad K., and Romain Hentges. “Association between tumor necrosis factor-α and disease progression in patients with multiple sclerosis.” New England Journal of Medicine 325.7 (1991): 467-472.] TNF-α levels were significantly higher among previous heart attack cases than controls (2.84 versus 2.57 pg/mL, P=0.02). The excess risk of recurrent coronary events after MI was predominantly seen among those with the highest levels of TNF-α, such that those with levels in excess of 4.17 pg/mL (the 95th percentile of the control distribution) had an ≈3-fold increase in risk. [Ridker, Paul M., et al. “Elevation of tumor necrosis factor-α and increased risk of recurrent coronary events after myocardial infarction.” Circulation 101.18 (2000): 2149-2153.] TNF-α was studied for its role in insulin resistance in 12 obese men with untreated Type 2 diabetes mellitus and in 6 age-and BMI-matched obese controls. Serum levels of TNF-α were higher in patients with insulin resistance (4.19±0.96 pg/ml) than in patients without insulin resistance (2.52±1.64 pg/ml) and in controls (2.03±1.21 pg/ml). Fasting serum concentrations of insulin were higher in patients with insulin resistance (16.2±5.0) than in patients without insulin resistance (7.3±2.2 IU/ml) and in controls (8.0±2.9 IU/ml). These data suggest that high levels of serum TNF-α in patients with insulin resistance are related to high levels of fasting insulin. The importance of the investigation was that the subjects recruited in the study were BMI matched, because human obesity is associated with an increased TNF-α mRNA expression in adipose tissue. [Mishima, Yasuo, et al. “Relationship between serum tumor necrosis factor-α and insulin resistance in obese men with Type 2 diabetes mellitus.” Diabetes research and clinical practice 52.2 (2001): 119-123.]

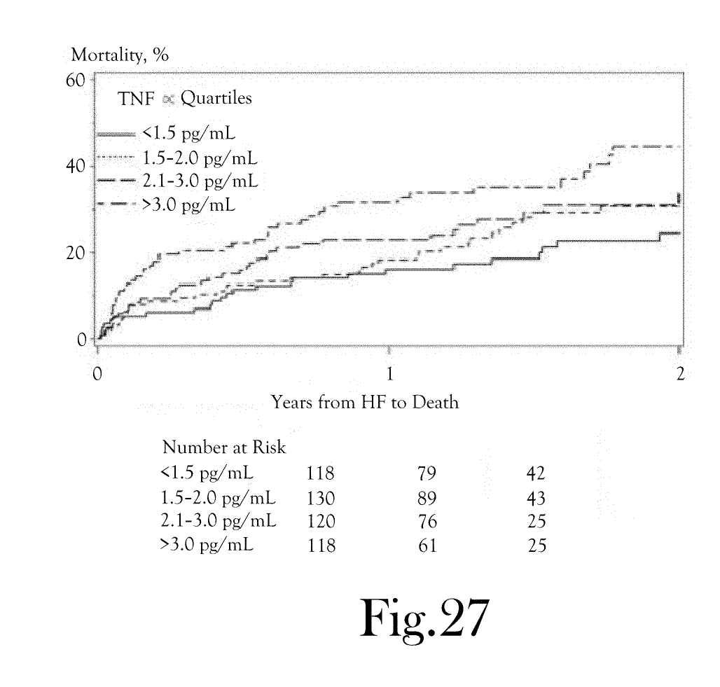

TNFα levels track with morbidity and mortality in a dose dependent manner, with the severity of chronic disease and death. A study demonstrated that serum TNFα is elevated in a large proportion of community heart failure patients with a wide range of ejection fraction and that elevated circulating TNFα was strongly associated with decreased creatinine clearance, anemia, and a high degree of comorbidity. Also, there is a strong independent association between elevated TNFα and mortality in heart failure patients regardless of ejection fraction. TNFα improves risk prediction in heart failure above traditional risk indicators. The unadjusted hazard ratios for death were 1.34 (95% CI 0.82 to 2.21), 1.47 (95% CI 0.89 to 2.44), and 2.10 (95% CI 1.30 to 3.38) from lowest to highest quartile, respectively, with the lowest quartile used as the referent. After adjustment for age, sex, EF, and comorbidities, this relationship held, with a hazard ratio for death of 1.88 (95% confidence interval, 1.09 to 3.25) in the highest versus lowest quartile (Ptrend across quartiles=0.028). The quartiles were: Quartile 1, TNFα<1.5 pg/mL; Quartile 2, 1.5≤TNFα<2.1 pg/mL; Quartile 3, 2.1 ≤TNFα<3.1 pg/mL; Quartile 4, TNFα≥3.1 pg/mL. Graphically, mortality risk in heart failure with TNFα is provided in FIG. 11. [Dunlay, Shannon M., et al. “Tumor Necrosis Factor-α and Mortality in Heart Failure A Community Study.” Circulation 118.6 (2008): 625-631.] In a community-based study of 3035 participants, a significant association between TNFRII and mortality risk was noted (HR 1.33, 1.19-1.49, p=<0.0001). [Schnabel R B, Yin X, Larson M G, Yamamoto J F, Fontes J D, Kathiresan S, et al. Multiple inflammatory biomarkers in relation to cardiovascular events and mortality in the community. Arterioscler Thromb Vasc Biol. 2013; 33:1728-33. doi: 10.1161/ATVBAHA.112.301174 PMID: 23640499]. FIG. 27 shows the Kaplan-Meier mortality curves by TNF-alpha quartile

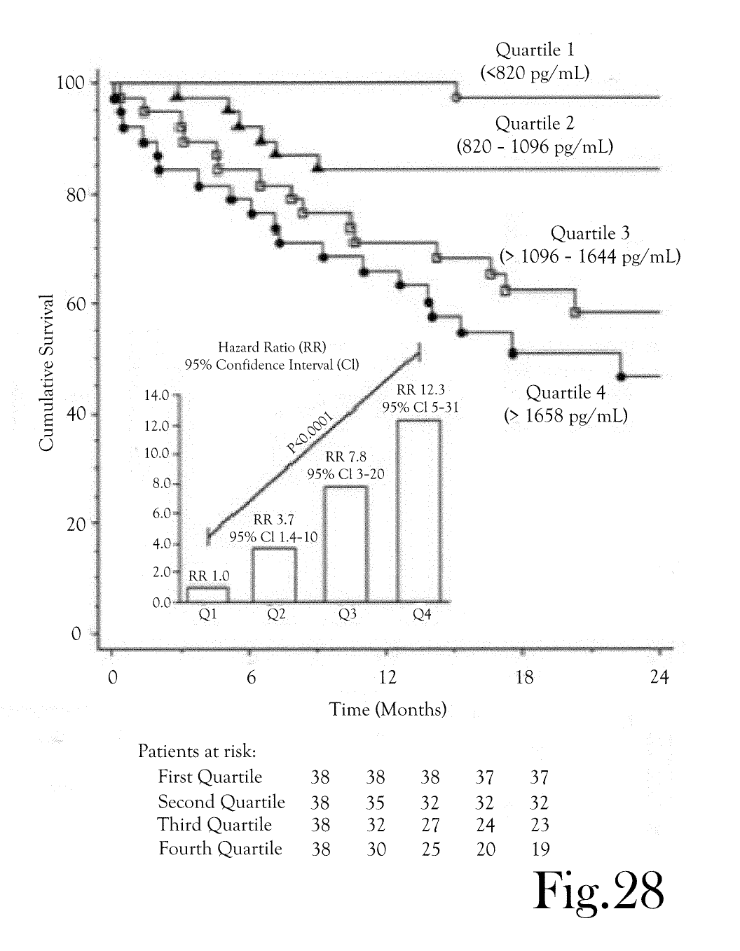

Increased plasma concentrations of cytokines and soluble cytokine receptors significantly predict impaired median to longer-term survival in patients with congestive heart failure. The best mortality predictive value and accuracy was found for sTNF-R1, a surrogate for TNFα, which provided the highest sensitivity and specificity among all immune parameters, independently of clinical variables and length of follow-up, FIG. 12. [Rauchhaus, Mathias, et al. “Plasma cytokine parameters and mortality in patients with chronic heart failure.” Circulation 102.25 (2000): 3060-3067.] FIG. 28 illustrates survival compared to TNF-alpha surrogate quartiles.

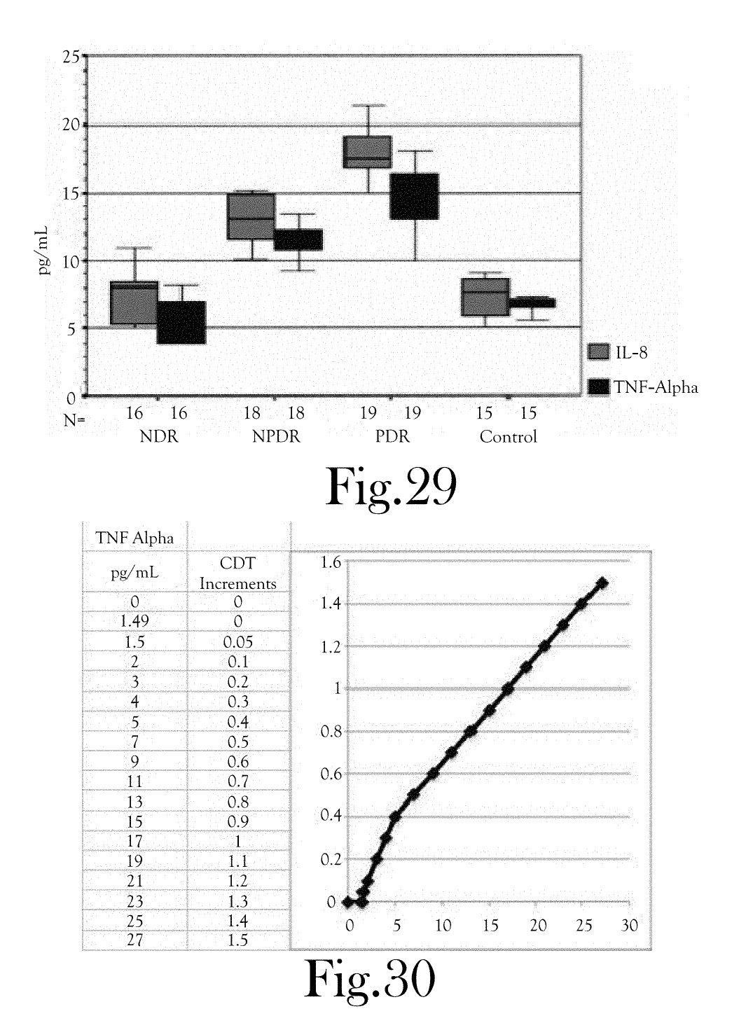

Progression of diabetic retinopathy (DR) from non-proliferative DR to proliferative DR is a serious complication of diabetes. This progression results in the activation and proliferation of vascular endothelial cells with leukocyte adhesion to the diabetic retinal vasculature. Overall, DR is characterized by a notable increase in antibody-dependent immune response. In addition, degeneration and loss of pericytes are seen as a result of systemic metabolic abnormalities associated with prolonged hyperglycemia. Increased serum TNF-α levels in diabetic patients shows a significant correlation between the levels and the grade of diabetic retinopathy. Mean serum levels of TNF according to stages of diabetic retinopathy are shown in FIG. 13 below. The level of the cytokine TNF is significantly higher in the more advanced stages of DR compared to controls. [Doganay, S., et al. “Comparison of serum NO, TNF-α, IL-1β, sIL-2R, IL-6 and IL-8 levels with grades of retinopathy in patients with diabetes mellitus.” Eye 16.2 (2002): 163-170.] FIG. 29 shows the mean serum IL-8 and TNF-alpha levels according to the stages of diabetic retinopathy (DR): no DR (NDR), non-proliferative DR (NPDR), proliferative DR (PDR) and controls.

TNFα elevation is more commonly associated with the following conditions: Alzheimer's disease, cancer, major depression, inflammatory bowel disease (IBD), multiple sclerosis, heart disease, diabetes, stroke, heart failure, kidney disease, chronic infections, hepatitis C, and chronic lower respiratory disease. Diseases of inflammation and aging are often associated with elevated TNFα including essentially every disease, the name of which ends in “itis.”

TNFα reference ranges vary and samples are obtained from serum. Quest Diagnostics: 1.2-15.3 pg/mL; ARUP laboratories: 22 pg/mL or less; Labcorp: <8.1 pg/mL

In exemplary embodiments, TNF values <1.5 pg/ml may be considered the upper limit for good health in most people. This is based on an increase in a myriad of chronic diseases with increased levels of the biomarker. Particularly, mortality in heart failure subjects increases with quartiles of TNF concentration.

A limited set of compounds have been shown to affect TNF-alpha concentrations in a subject. However, as with most cytokines, direct measures to reduce their levels appears to do more harm than good. Appropriate strategies of TNF management include lifestyle and particularly dietary management that augment immune function and reduce inflammation.

In various embodiments, TNF-alpha contributes to a subject's chronic disease temperature as follows: Total maximum contribution to the CDT calculation is 1.5° F. (0.83° C.). See FIG. 30.

Beta-2-microglobulin

In various embodiments beta-2-microglobulin (B2M) may be used as a biomarker. One of the important functions of the human immune system is distinguishing self from nonself molecules. Most nucleated cells in the human body carry class I antigens that help the immune system identify self-molecules. These antigens have a heavy chain and an associated light chain. This light-protein chain, which can be shed into serum, is beta 2-microglobulin. The molecule was discovered initially as a serum protein. B2M is an 11.8-kD protein which forms one of the chains of the major histocompatibility complex (MHC) class I molecule normally present on the surface of every nucleated cell in the human body. This protein further functions to present antigens to cytotoxic T lymphocytes that are carrying out surveillance for infection. [Nakamuro K, Tanigaki N, Pressman D. Multiple common properties of human beta2-microglobulin and the common portion fragment derived from HL-A antigen molecules. Proc Natl Acad Sci USA. 1973 October. 70(10):2863-5]

Under physiologic conditions, B2M is produced at a constant rate and is eliminated from circulation by kidneys. In patients with a range of inflammatory, hematologic, immunodeficiency, and renal diseases, plasma B2M levels are elevated [Sedighi O, Abediankenari S, Omranifar B. Association Between Plasma Beta-2 Microglobulin Level and Cardiac Performance in Patients With Chronic Kidney Disease. Nephro-urology Monthly. 2015; 7(1):e23563.]

Serum and plasma B2M values have emerged as markers for the activation of the cellular immune system, as well as a tumor marker in certain hematologic malignancies. Urine B2M values indicate renal filtration disorders. Measurement of values in both serum and urine can help distinguish a problem of cellular activation from a renal disorder. [Bethea M, Forman DT. Beta 2-microglobulin: its significance and clinical usefulness. Ann Clin Lab Sci. 1990 May-June 20(3):163-8]. In subjects with chronic kidney disease, plasma B2M levels are elevated, especially in patients on hemodialysis in whom glomerular filtration rate is almost completely absent. B2M is also a surrogate marker of middle-molecular-weight uremic toxins in patients on hemodialysis, which is cleared only by high-flux membrane. In some studies, predialysis serum B2M level predicted mortality and increase of B2M clearance during hemodialysis was associated with improved outcomes. In addition, elevated plasma B2M level is a potential risk factor for the development of dialysis-related amyloidosis.

Low serum levels of B2M essentially indicate decreased disease activity in conditions for which B2M is used as a prognostic marker (multiple myeloma, lymphoma, leukemia) or the absence of such a disease process. However, low B2M levels are never used to rule out a particular disease (eg, lymphoma) in the absence of other more definitive tests. [Durie B G, Stock-Novack D, Salmon S E, Finley P, Beckord J, Crowley J, et al. Prognostic value of pretreatment serum beta 2 microglobulin in myeloma: a Southwest Oncology Group Study. Blood. 1990 Feb. 15. 75(4):823-301.]

Increased serum B2M levels reflect increased activity of a disease process and can be a sensitive marker for this purpose in many hematologic disorders. The absolute value is less important than the historical values, except in certain situations such as multiple myeloma, in which a value of less than 4 μg/mL was found to correlate with increased survival. [Durie B G, Stock-Novack D, Salmon S E, Finley P, Beckord J, Crowley J, et al. Prognostic value of pretreatment serum beta 2 microglobulin in myeloma: a Southwest Oncology Group Study. Blood. 1990 Feb. 15. 75(4):823-30.]

Increased CSF B2M levels are seen in certain conditions such as multiple sclerosis, AIDS dementia complex, and meningeal spread of hematologic tumors. [Adachi N. Beta-2-microglobulin levels in the cerebrospinal fluid: their value as a disease marker. A review of the recent literature. Eur Neurol. 1991. 31(4):181-5]. B2M is shed from the surface of nucleated cells into serum; increased levels can be seen in a wide variety of disorders that involve increased cell turnover and/or activation of the immune system. Whereas this makes B2M a marker for myriad diseases, it also makes it a relatively nonspecific marker. This has led to its use as a quantitative prognostic marker much more than as a diagnostic marker. Despite this limitation, B2M is often part of the initial panels for certain diseases (multiple myeloma, Waldenström macroglobulinemia, myelodysplastic syndromes) in which the baseline value of B2M affects staging, prognosis, and treatment.

In one embodiment, B2M is associated with the genesis and proliferation of diseases including:

Malignancies: Significantly elevated levels of B2M can be found in multiple myeloma, malignant lymphomas, and chronic lymphocytic leukemia. Values have been shown to correlate with prognosis. In multiple myeloma, serum values of less than 4 μg/mL were associated with significant increase in survival. Serum CRP is independent of serum B2M in multiple myeloma prognostication. This feature allowed stratification of multiple myeloma patients into 3 groups according to CRP and beta 2M serum levels: (1) low risk group, CRP and B2M less than 6 mg/L (50% of patients); (2) intermediate risk group, CRP or B2M greater than or equal to 6 mg/L (35% of patients); (3) high risk group, CRP and B2M greater than or equal to 6 mg/L (15% of patients). Survival was 54, 27, and 6 months, respectively (P less than 0.0001). [Bataille, Regis, et al. “C-reactive protein and beta-2 microglobulin produce a simple and powerful myeloma staging system.” Blood 80.3 (1992): 733-737.]

Serum B2M <4 mcg/mL is a good prognostic factor in patients with multiple myeloma. In a study of pretreatment serum B2M levels in 100 patients with myeloma it was reported that the median survival of patients with values >4 mcg/mL was 12 months, whereas median survival for patients with values <4 mcg/mL was 43 months.

Renal diseases: B2M accumulates in the serum of individuals with renal failure. Although decreased clearance appears to be the primary reason for elevation of B2M levels in persons with end-stage renal disease, it has been postulated that the uremic state may result in increased production of the molecule. Plasma B2M level was elevated in patients with chronic kidney disease and this level progressively increases with decreasing GFR. Moreover, plasma B2M level is associated with some metabolic and cardiac performance factors in predialysis CKD patients, Table 18.

TABLE 18
Clinical and Biochemical Characteristics of the Study Population

| | Group I | Group II | P |
|---|---|---|---|
| Parameter | (n = 86) | (n = 78) | Value |
| Age, y | 62.17 ± 16.52 | 58.61 ± 9.62 | 0.114 |
| Gender | | | 0.732 |
| Male | 46 | 41 | |
| Female | 40 | 37 | |
| BMI, kg/m² | 22.14 ± 3.66 | 24.72 ± 6.18 | 0.641 |
| Serum Cr, μmol/L | 195.36 ± 68.95 | 76.02 ± 18.56 | <0.001 |
| GFR, mL/min | 48.2 ± 17.3 | 102.8 ± 31.6 | <0.001 |
| Hemoglobin, g/L | 112 ± 23.2 | 142 ±35.2 | 0.002 |
| Serum Calcium, mmol/L | 2.29 ± 0.58 | 2.43 ± 1.10 | 0.173 |
| Serum Phosphate, mmol/L | 1.52 ± 0.39 | 1.39 ± 0.84 | 0.165 |
| Albumin, g/L | 31.8 ± 6.6 | 47.7 ± 12.3 | 0.012 |
| C-Reactive Protein, | 64.76 ± 43.81 | 29.52 ± 24.76 | 0.002 |
| nmol/dL | | | |
| Total Cholesterol, mmol/L | 5.99 ± 1.10 | 5.66 ± 1.86 | 0.621 |
| LDL-Cholesterol, mmol/L | 3.49 ± 0.84 | 3.14 ± 0.21 | 0.452 |
| Triglycerides, mmol/L | 2.68 ± 0.50 | 2.54 ± 0.35 | 0.663 |
| Beta-2 Microglobulin, | 7.6 ± 3.7 | 2.1 ± 1.7 | <0.001 |
| mg/L | | | |

Group 1: clinical CKD—chronic kidney disease; Group 2: healthy controls. [Sedighi, Omid, Saeid Abediankenari, and Batoul Omranifar. “Association Between Plasma Beta-2 Microglobulin Level and Cardiac Performance in Patients With Chronic Kidney Disease.” Nephro-urology monthly 7.1 (2015).]

Neurologic diseases: Elevated CSF B2M levels correlate with disease activity in multiple sclerosis, neuro-Behçet disease, sarcoidosis, AIDS dementia complex, and meningeal dissemination of malignant hematologic malignancies. Aging is a major risk factor for cognitive decline and neurodegenerative diseases. B2M is now identified as a blood-borne factor that detrimentally influences the brain during the aging process. [Filiano, Anthony J., and Jonathan Kipnis. “Breaking bad blood:[beta] 2-microglobulin as a pro-aging factor in blood.” Nature medicine 21.8 (2015): 844-845.] The absence of endogenous B2M expression abrogates age-related cognitive decline and enhances neurogenesis in aged mice. [Smith, Lucas K., et al. “[beta] 2-microglobulin is a systemic pro-aging factor that impairs cognitive function and neurogenesis.” Nature medicine 21.8 (2015): 932-937.]

Rheumatologic disease: Ankylosing spondylitis may be caused by deposition of B2M within the joints.

Cardiovascular disease: Higher B2M levels are independently associated with overall and cardiovascular mortality and cardiovascular events, particularly in subjects with renal dysfunction. In a study 359 major cardiovascular events in 271 (27%) patients were noted. B2M was significantly associated with the occurrence of major adverse cardiovascular events. With increasing quartiles of B2M, the adjusted hazard ratios were 1.19 (95% CI, 0.81 to 1.73), 1.51 (95% CI, 1.05 to 2.18), and 1.88 (95% CI, 1.26 to 2.79) compared with the lowest quartile, respectively (P<0.001). Adjusted hazard ratios for the occurrence of death, myocardial infarction, and stroke for increasing quartiles of B2M were 1.25 (95% CI, 0.92 to 1.70), 1.52 (95% CI, 1.12 to 2.06), and 1.62 (95% CI, 1.16 to 2.67) compared with the lowest quartile, respectively (P<0.001). Through statistical estimation of improvement in risk stratification, addition of B2M to baseline risk factors improved the risk stratification for major cardiovascular events, at least as much as high-sensitivity C-reactive protein or even better. [Amighi, Jasmin, et al. “Beta 2 microglobulin and the risk for cardiovascular events in patients with asymptomatic carotid atherosclerosis.” Stroke 42.7 (2011): 1826-1833.], FIG. 14, 15.

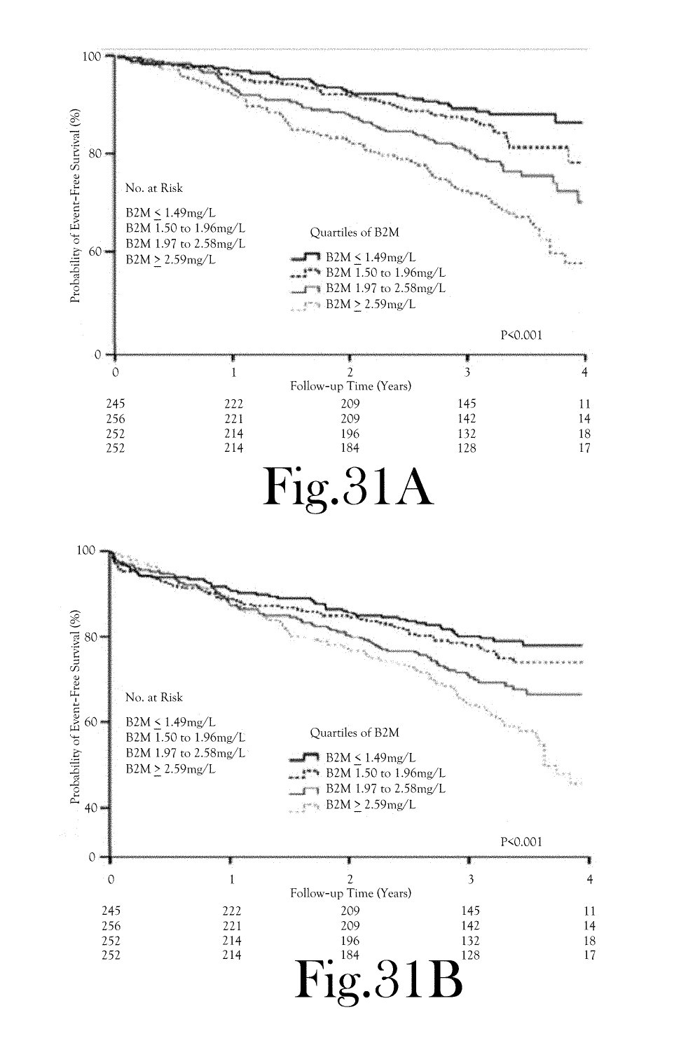

FIG. 31A shows the Kaplan-Meier estimates for major adverse cardiovascular events (composite of myocardial infarction, percutaneous coronary interventions, coronary bypass graft, stroke, and death) according to quartiles of beta 2 microglobulin (B2M). FIG. 31B shows the Kaplan-Meier estimates for death, myocardial infarction, and stroke according to quartiles of beta 2 microglobulin (B2M)

Reference Range: Serum and plasma B2M values have emerged as markers for the activation of the cellular immune system, as well as a tumor marker in certain hematologic malignancies. Urine B2M values indicate renal filtration disorders. Measurement of values in both serum and urine can help distinguish a problem of cellular activation from a renal disorder. [Bethea M, Forman D T. Beta 2-microglobulin: its significance and clinical usefulness. Ann Clin Lab Sci. 1990 May-June 20(3):163-81.]

The reference range of beta2 microglobulin in urine samples is 0-0.3 μg/mL. In serum or plasma samples, the reference range is 0-3 μg/mL.

Reference Values: 1.21-2.70 mcg/mL

Collection and panels: Beta2 microglobulin can be determined in urine, serum, or plasma samples. It is not necessary to draw the sample in a fasting state, and no special preparations are necessary. Blood is collected by venipuncture in a red-top tube and centrifuged to separate serum from cells after clot formation. Samples may be stored refrigerated at 2-8° C. for 5 days. For longer storage (up to 6 months), samples should be stored frozen at −20° C. To avoid repeated thawing and freezing, the samples should be aliquoted. Bilirubin and hemolysis do not significantly affect the procedure. However, gross lipemia can interfere with results.

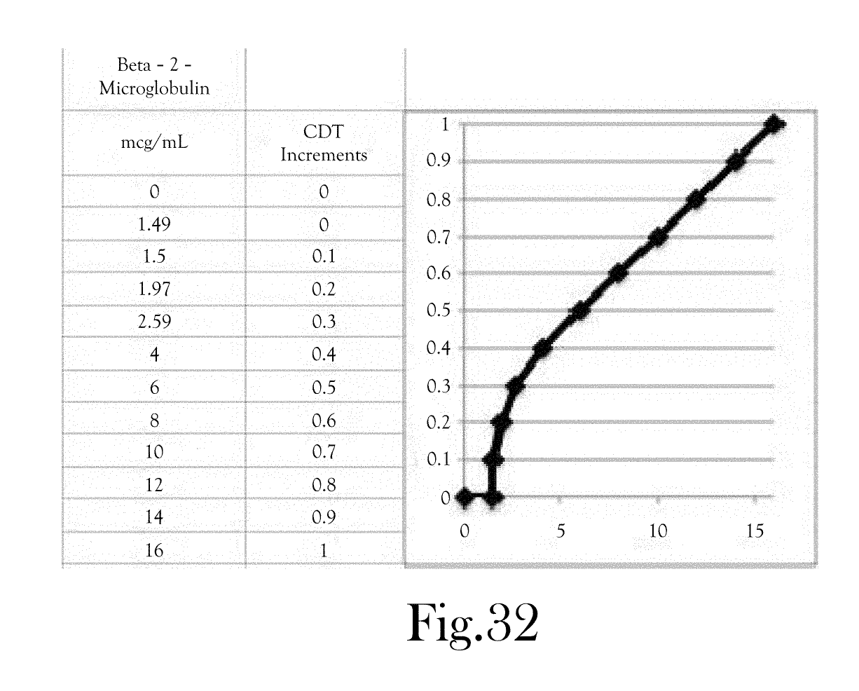

In various embodiments, beta-2-microglobulin contributes to a subject's chronic disease temperature as follows: Total maximum contribution to the CDT calculation is 1.0° F. (0.56° C.). See FIG. 32.

Myeloperoxidase

Myeloperoxidase (MPO) is an enzyme linked to both inflammation and oxidative stress. It is abundantly expressed in the azurophilic granules of most leukocyte subspecies, including neutrophils and monocytes (3). MPO is released by leukocytes in a state of inflammation and catalyzes the formation of several reactive species, including hypochlorous acid, and thus has a role in host defense against microorganisms (3). epidemiological studies has shown that higher concentrations of MPO are associated with an increased CVD risk, independent of classical CVD risk factors. [Schindhelm, Roger K., et al. “Myeloperoxidase: a useful biomarker for cardiovascular disease risk stratification?.” Clinical chemistry 55.8 (2009): 1462-1470.]

Inflammation and oxidative stress are associated with atherosclerosis. Myeloperoxidase (MPO) is linked to both inflammation and oxidative stress by its location in leukocytes and its role in catalyzing the formation of oxidizing agents. Recent evidence suggests that MPO activity precipitates atherogenesis. Measurement of MPO in plasma may therefore contribute to cardiovascular disease (CVD) risk stratification.

MPO is an important marker for cardiovascular diseases. Blood and leukocyte MPO activity are higher in patients with CAD than angiographically verified normal controls, and this increased activity is significantly associated with presence of CAD (odds ratio, 11.9; 95% confidence interval (CI), 5.5-25.5). Results are independent of the patient's age, sex, hypertension, smoking, or diabetes status, LDL concentration, leukocyte count, and Framingham global risk score. MPO was measured in baseline samples of a case control study nested in the prospective EPIC-Norfolk population study: case subjects (n=1138) were apparently healthy men and women who developed CAD during 8 years of follow-up; control subjects (n=2237) matched for age, gender, and enrollment time, remained free of CAD. The MPO levels were significantly higher in case subjects than in control subjects and correlated with C-reactive protein (CRP) and white blood cell count. Risk of future CAD increased in consecutive quartiles of MPO concentration, with an odds ratio (OR) of 1.49 in the top versus bottom quartile (MPO range, pmol/l quartile 1: <454, quartile 2: 454-638, quartile 3: 638-951, quartile 4: >951). After adjustment for traditional risk factors, the OR in the top quartile remained significant at 1.36 (95% CI 1.07 to 1.73). Of interest in this study, serum MPO levels were associated with the risk of future development of CAD in apparently healthy individuals, but the association was weaker than that of traditional risk factors and CRP. However MPO, at variance from CRP, was largely independent from classical risk factors.

The potential usefulness for risk stratification of blood concentrations of MPO was examined in 1090 patients with acute coronary syndrome (ACS). Rates of death and myocardial infarction (MI) were determined at 6 months of follow-up. An MPO cutoff of 350 μg/L was associated with an adjusted hazard ratio was 2.25 (95% CI, 1.32-3.82). The effects were particularly impressive in patients with undetectable cardiac troponin T (cTnT<0.01 μg/L), in whom the hazard ratio was 7.48 (95% CI, 1.98-28.29). Of interest, the increase in risk was already evident after 72 hours, increasing only slightly thereafter. MPO presents the insightful characteristic of at variance from other inflammatory markers commonly used (as CRP, fibrinogen) that remain elevated for relatively long time or have an extremely short and unreliable half-life (such as interleukins). The predictive value of MPO was independent by C-reactive protein and high MPO serum levels indicated increased cardiac risk both in patients with medium C-reactive protein serum levels (20.0% versus 5.9%; P<0.001) and in those with low C-reactive protein serum levels (17.8% versus 0%; P<0.001), suggesting that recruitment and degranulation of neutrophils is a primary event and is followed by release of other systemic mediators and acute-phase proteins such as C-reactive protein. Taken together, these data suggest that CRP and MPO may be complementary and explore different fields: CRP is a marker of disease activity and vascular inflammation, and is useful for long-term risk stratification while MPO is a marker of plaque instability and neutrophil activation and may be associated with short-term stratification, in particular in patients with troponin negative levels. [Loria, Valentina, et al. “Myeloperoxidase: a new biomarker of inflammation in ischemic heart disease and acute coronary syndromes.” Mediators of inflammation 2008 (2008).]

MPO plasma concentrations were determined in 3036 participants of the Ludwigshafen Risk and Cardiovascular Health study (median follow-up 7.75 years). MPO concentrations were positively associated with age, diabetes, smoking, markers of systemic inflammation (interleukin-6, fibrinogen, C-reactive protein, serum amyloid A) and vascular damage (vascular cellular adhesion molecule-1 and intercellular adhesion molecule-1) but negatively associated with HDL-cholesterol and apolipoprotein A-I. After adjustment for cardiovascular risk factors MPO concentrations in the highest versus the lowest quartile were associated with a 1.34-fold risk (95% CI: 1.09-1.67) for total mortality. In the adjusted model the hazard ratio for cardiovascular mortality in the highest MPO quartile was 1.42 (95% CI: 1.07-1.88). MPO levels in ng/ml in the quartiles are: quartile 1: <21, quartile 2: 21-30, quartile 3: 31-45, quartile 4: >45, FIG. 16. [Scharnagl, Hubert, et al. “Association of myeloperoxidase with total and cardiovascular mortality in individuals undergoing coronary angiography—The LURIC study.” International journal of cardiology 174.1 (2014): 96-105.]

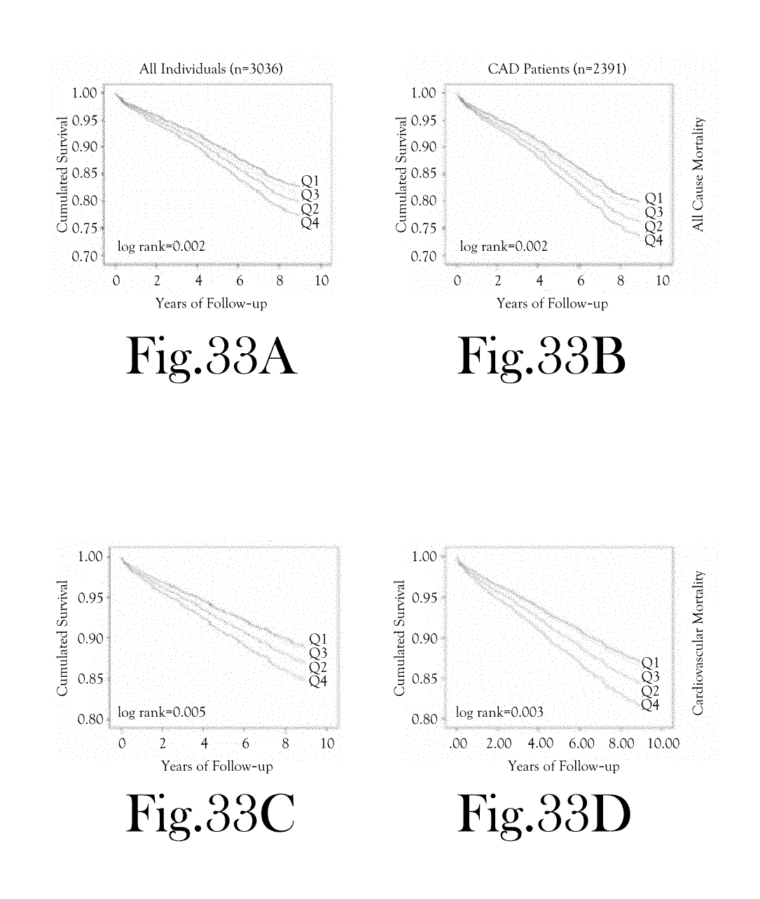

FIGS. 33A-D show the association of myeloperoxidase with total and cardiovascular mortality in individuals undergoing coronary angiography—The LURIC study

Reference Ranges: Myeloperoxidase (MPO). MPO is an enzyme made by white blood cells in the artery wall. Elevated levels indicate unstable plaque and a high risk of having a near term cardiac event (within one to six months).

Reference Ranges: Optimal: <350 pmol/L; Borderline 350-633 pmol/L; High >633 pmol/L. Reference values apply to all ages.

In exemplary embodiments, MPO values <210 pmol/L may be considered the upper limit for good health in all people. This is based on an increased risk of vascular and inflammatory diseases of the heart and increased incidence of mortality.

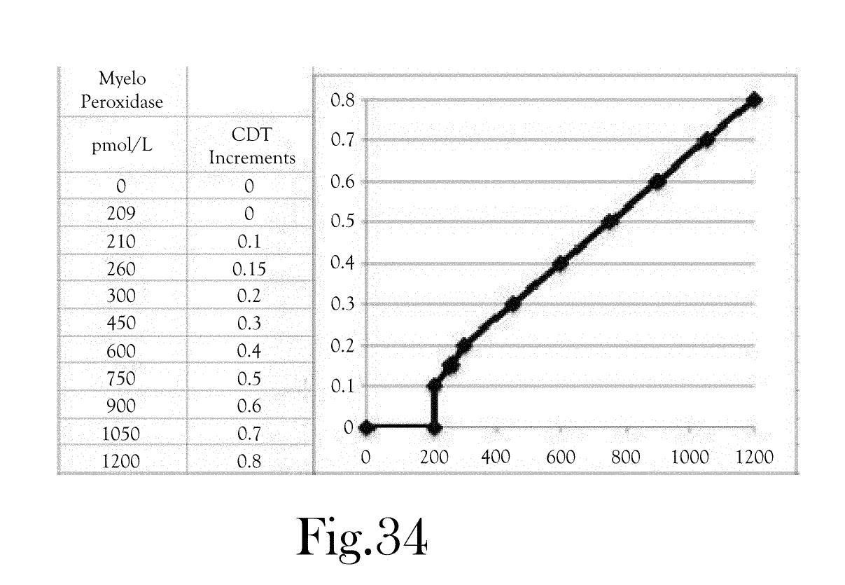

In various embodiments, myeloperoxidase contributes to a subject's chronic disease temperature as follows: Total maximum contribution to the CDT calculation is 0.8° F. (0.44° C.). See FIG. 34.

N-Terminal pro Brain Natriuretic Peptide (NT-proBNP)

In various embodiments, NT-proBNP is used as a biomarker. B-type natriuretic peptide (brain natriuretic peptide: BNP) is a small, ringed peptide secreted by the heart to regulate blood pressure and fluid balance. This peptide is stored in and secreted predominantly from membrane granules in the heart ventricles in a pro form (proBNP). Once released from the heart in response to ventricle volume expansion and/or pressure overload, the N-terminal (NT) piece of 76 amino acids (NT-proBNP) is rapidly cleaved by the enzymes corin and/or furin to release the active 32 amino acid peptide (BNP). Both BNP and NT-proBNP are markers of atrial and ventricular distension due to increased intracardiac pressure. The New York Heart Association (NYHA) developed a 4-stage functional classification system for congestive heart failure (CHF) based on the severity of the symptoms. Studies have demonstrated that the measured concentrations of circulating BNP and/or NT-proBNP increase with the severity of CHF based on the NYHA classification.

Natriuretic peptides are produced primarily within the heart and released into the circulation in response to increased wall tension. Brain natriuretic peptide (BNP), in contrast to atrial natriuretic peptide (ANP), is not only secreted from the atria but also from the ventricles, especially in patients with heart failure. Circulating concentrations of several cardiac natriuretic peptides—including ANP, BNP, and their N-terminal pro-hormones (N-terminal pro-atrial natriuretic peptide (NT-proANP) and N-terminal pro-brain natriuretic peptide (NT-proBNP)) are raised in both symptomatic and asymptomatic patients with left ventricular dysfunction. Studies suggest that BNP and NT-proBNP may be superior to ANP and NT-proANP in the detection of left ventricular dysfunction. A reliable and less time consuming enzyme linked immunosorbent assay (ELISA) method for the analysis of NT-proBNP has been developed and NT-proBNP may therefore be a suitable peptide for a diagnostic assay. [Bay, M., et al. “NT-proBNP: a new diagnostic screening tool to differentiate between patients with normal and reduced left ventricular systolic function.” Heart 89.2 (2003): 150-154.]

In subjects with acute coronary syndrome, baseline NT-proBNP levels >250 ng/L were associated with higher event rates. In patients with high NT-proBNP baseline levels, lack of a rapid decline in NT-proBNP levels (≤250 ng/L) was linked to an adverse short-term prognosis. In patients with low NT-proBNP baseline levels, a rise in NT-proBNP levels over 72 hours to >250 ng/L was also linked to an adverse 30-day prognosis. [Heeschen, Christopher, et al. “N-terminal pro-B-type natriuretic peptide levels for dynamic risk stratification of patients with acute coronary syndromes.” Circulation 110.20 (2004): 3206-3212.]

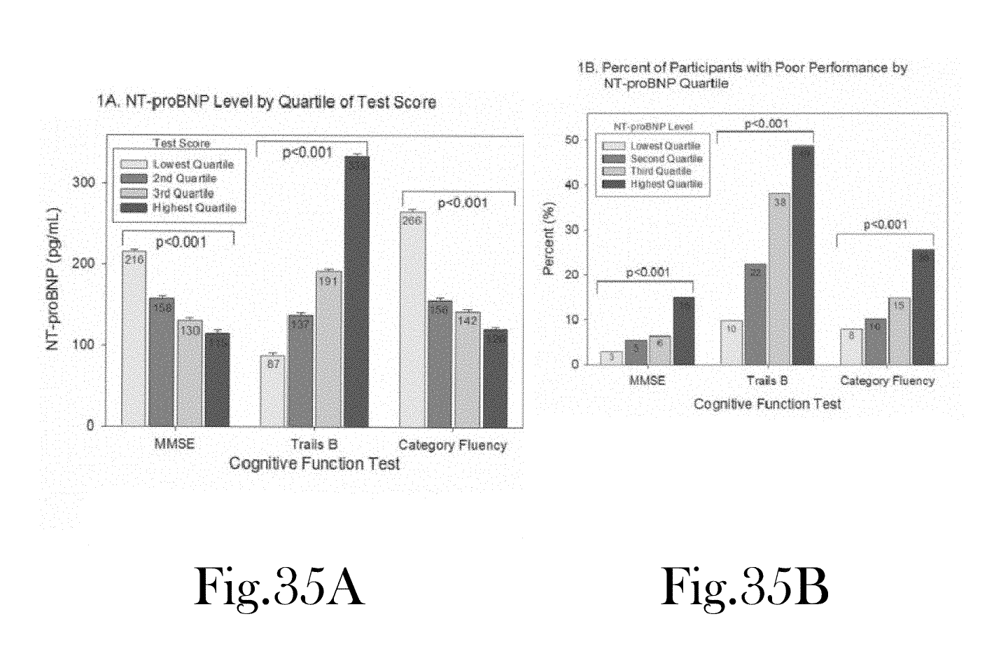

Elevated NT-proBNP levels are associated with poor cognitive function in older adults. In a study of 950 men and women, participants with high NT-proBNP levels (≥450 pg/mL, n=198) were older and had a higher prevalence of coronary heart disease (12% vs. 30%), and stroke (5% vs. 11%) (both p's≤0.001). In unadjusted analyses, cognitive function test scores were significantly associated with NT-proBNP levels (p<0.001). After adjusting for age, sex, education, hypertension, body mass index, exercise, alcohol use, smoking, low density lipoprotein cholesterol, creatinine clearance, and prior cardiovascular disease, elevated NT-proBNP levels remained independently associated with poor cognitive performance on MMSE (odds ratio [95% confidence interval] 2.0 [1.1-3.6], p=0.02) and Trails B (1.7 [1.2-2.7], p=0.01), but not Category Fluency (1.4 [0.9-2.2], p=0.19). Results were unchanged after excluding the 6% of participants with a history of stroke. NT-proBNP levels were strongly and independently associated with poor cognitive function, FIG. 17. [Daniels, Lori B., et al. “Elevated natriuretic peptide levels and cognitive function in community-dwelling older adults.” The American journal of medicine 124.7 (2011): 670-e1.] FIG. 35A shows theT-proBNP Level by Quartile of Test Score; FIG. 35B shows the percent of Participants with Poor Performance by NT-proBNP Quartile.

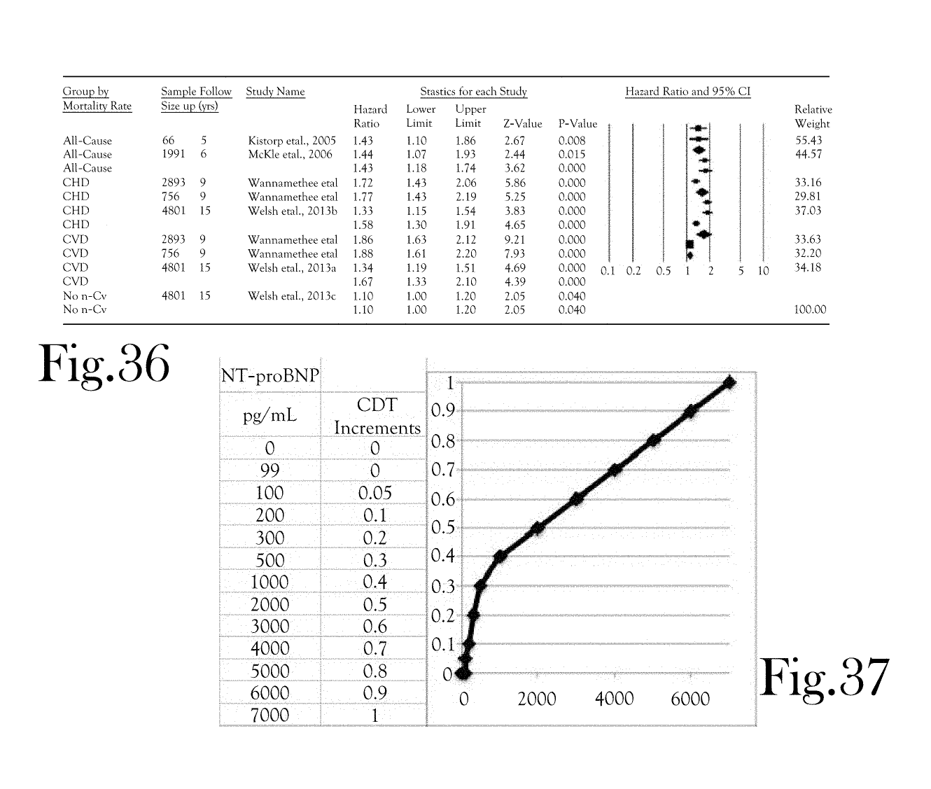

Overall, higher concentrations of NT-proBNP at baseline were associated with greater subsequent mortality, see FIG. 36. Examination of the relationships between NT-proBNP and all-cause mortality risk reveals a concentration dependant association of NT-proBNP with greater risk of all-cause mortality (HR 1.43, 1.18-1.74, p<0.0001; I2=0; Q 0.001; DF 1; p=0.97), CHD mortality (HR 1.58, 1.30-1.91, p<0.0001; I2=71; Q 6.93; DF 2; P=0.031) and CVD mortality (HR 1.67, 1.33-2.10, p<0.0001; I2=88; Q 16.88; DF 2; p=0.0002). One study reported an association with non-CVD mortality. [Barron, Evelyn, et al. “Blood-borne biomarkers of mortality risk: systematic review of cohort studies.” PloS one 10.6 (2015): e0127550.]

In a study of nearly 100-subjects, a total of 256 participants (26.2%) had a cardiovascular event or died. Each increasing quartile of NT-proBNP level (range of quartile 1, 8.06-73.95 pg/mL; quartile 2, 74-174.5 pg/mL; quartile 3, 175.1-459 pg/mL; quartile 4, ≥460 pg/mL) was associated with a greater risk of cardiovascular events or death, ranging from 23 of 247 (annual event rate, 2.6%) in the lowest quartile to 134 of 246 (annual event rate, 19.6%) in the highest quartile (unadjusted hazard ratio [HR] for quartile 4 vs quartile 1, 7.8; 95% confidence interval [CI], 5.0-12.1; P<0.001). Each standard deviation increase in log NT-proBNP level (1.3 pg/mL) was associated with a 2.3-fold increased rate of adverse cardiovascular outcomes (unadjusted HR, 2.3; 95% CI, 2.0-2.6; P<0.001), and this association persisted after adjustment for all of the other prognostic measures (adjusted HR, 1.7; 95% CI, 1.3-2.2; P<0.001). The addition of NT-proBNP level to standard clinical assessment and complete echocardiographic parameters significantly improved the area under the ROC curves for predicting subsequent adverse cardiovascular outcomes (0.80 for clinical risk factors and echocardiographic parameters plus log NT-proBNP vs 0.76 for clinical risk factors and echocardiographic parameters only; P=0.006).

Reference Values

<50 years of age

NT-proBNP values <300 pg/mL have a 99% negative predictive value for excluding acute congestive heart failure (CHF). A cutoff of 1,200 pg/mL for patients with an eGFR <60 yields a diagnostic sensitivity and specificity of 89% and 72% for acute CHF. NT-proBNP values >450 pg/mL are consistent with CHF in adults under 50 years of age.

50-75 years of age

NT-proBNP values <300 pg/mL have a 99% negative predictive value for excluding acute CHF. A cutoff of 1,200 pg/mL for patients with an eGFR <60 yields a diagnostic sensitivity and specificity of 89% and 72% for acute CHF. A diagnostic NT-proBNP cutoff of 900 pg/mL has been suggested in adults 50 to 75 years of age in the absence of renal failure.

>75 years of age

NT-proBNP values <300 pg/mL have a 99% negative predictive value for excluding acute CHF. A cutoff of 1,200 pg/mL for patients with an eGFR <60 yields a diagnostic sensitivity and specificity of 89% and 72% for acute CHF. A diagnostic NT-proBNP cutoff of 1,800 pg/mL has been suggested in adults over 75 years of age in the absence of renal failure.

NT-Pro BNP levels are loosely correlated with New York Heart Association (NYHA) functional class, Table 19. [Alhusseiny, Adil Hassan, et al. “Heart Failure: Discrepancy Between NYHA Functional Classification, Serum NT-pro Brain Natriuretic Peptide and Ejection Fraction.” Eur J Gen Med 10.1 (2013): 26-31.]

TABLE 19
Distribution of cases according to the NYHA classification
and their corresponding serum level of NT-proBNP.

| | Serum NT-proBNP | Ejection fraction |
|---|---|---|
| NYHA classification | (pg/ml) | (%) |
| Healthy subjects (n = 24) | 76.3 ± 99.0 | |
| Class 1 (mild)(n = 57) | 878.1 ± 1090.1 | 55.43 ± 8.48 |
| Class 2 (mild)(n = 65) | 1418.2 ± 3197.7 | 52.88 55.43 ± 8.03 |
| Class 3 (moderate)(n = 33) | 3969.5 ± 4168.8 | 48.22 55.43 ± 10.22 |
| Class 4 (severe)(n = 14) | 8270.2 ± 6116.9 | 43.42 55.43 ± 14.58 |

In various embodiments, NT-proBNP contributes to a subject's chronic disease temperature as follows: Total maximum contribution to the CDT calculation is 1.0° F. (0.56° C.). See FIG. 37.

Cystatin C

In various embodiments, Cystatin C is used as a biomarker. Cystatin C or cystatin 3 (formerly gamma trace, post-gamma-globulin or neuroendocrine basic polypeptide), a protein encoded by the CST3 gene, is mainly used as a biomarker of kidney function. Cystatin C is a low molecular weight (13,250 kD) cysteine proteinase inhibitor that is produced by all nucleated cells and found in body fluids, including serum. Since it is formed at a constant rate and freely filtered by the kidneys, its serum concentration is inversely correlated with the glomerular filtration rate (GFR); that is, high values indicate low GFRs while lower values indicate higher GFRs, similar to creatinine. Recently, it has been studied for its role in predicting new-onset or deteriorating cardiovascular disease. It also seems to play a role in brain disorders involving amyloid (a specific type of protein deposition), such as Alzheimer's disease. In humans, all cells with a nucleus (cell core containing the DNA) produce cystatin C as a chain of 120 amino acids. It is found in virtually all tissues and body fluids. It is a potent inhibitor of lysosomal proteinases (enzymes from a special subunit of the cell that break down proteins) and probably one of the most important extracellular inhibitors of cysteine proteases (it prevents the breakdown of proteins outside the cell by a specific type of protein degrading enzymes). Cystatin C belongs to the type 2 cystatin gene family.

Cystatin C may be used as an alternative to creatinine and creatinine clearance to screen for and monitor kidney dysfunction in those with known or suspected kidney disease. It may be especially useful in those cases where creatinine measurement is not appropriate, for instance, in those who have liver cirrhosis, are very obese, are malnourished, or have reduced muscle mass. Measuring cystatin C may also be useful in the early detection of kidney disease when other test results may still be normal and an affected person may have few, if any, symptoms.

Corticosteroids can increase levels cystatin C levels while cyclosporine can decrease them. Cystatin C has been associated with hyperhomocysteinemia (increased homocysteine), which is often found in kidney transplant patients, and it has been shown to increase with the progression of liver disease.

The Cardiovascular Health Study (CHS) is a community-based, longitudinal study of adults who were 65 years of age or older at the study's inception. Its main purpose is to evaluate risk factors for the development and progression of cardiovascular disease in elderly persons. Creatinine and cystatin C were measured in serum samples collected from 4637 participants at the study visit in 1992 or 1993; follow-up continued until Jun. 30, 2001. For each measure, the study population was divided into quintiles, with the fifth quintile subdivided into thirds (designated 5a, 5b, and 5c). Higher cystatin C levels were directly associated, in a dose response manner, with a higher risk of death from all causes. As compared with the first quintile, the hazard ratios (and 95 percent confidence intervals) for death were as follows: second quintile, 1.08 (0.86 to 1.35); third quintile, 1.23 (1.00 to 1.53); fourth quintile, 1.34 (1.09 to 1.66); quintile 5a, 1.77 (1.34 to 2.26); 5b, 2.18 (1.72 to 2.78); and 5c, 2.58 (2.03 to 3.27). In contrast, the association of creatinine categories with mortality from all causes appeared to be J-shaped. As compared with the two lowest quintiles combined (cystatin C level, 0.99 mg per liter), the highest quintile of cystatin C (1.29 mg per liter) was associated with a significantly elevated risk of death from cardiovascular causes (hazard ratio, 2.27), myocardial infarction (hazard ratio, 1.48), and stroke (hazard ratio, 1.47) after multivariate adjustment. The fifth quintile of creatinine, as compared with the first quintile, was not independently associated with any of these three outcomes. [Shlipak, Michael G., et al. “Cystatin C and the risk of death and cardiovascular events among elderly persons.” New England Journal of Medicine 352.20 (2005): 2049-2060.]

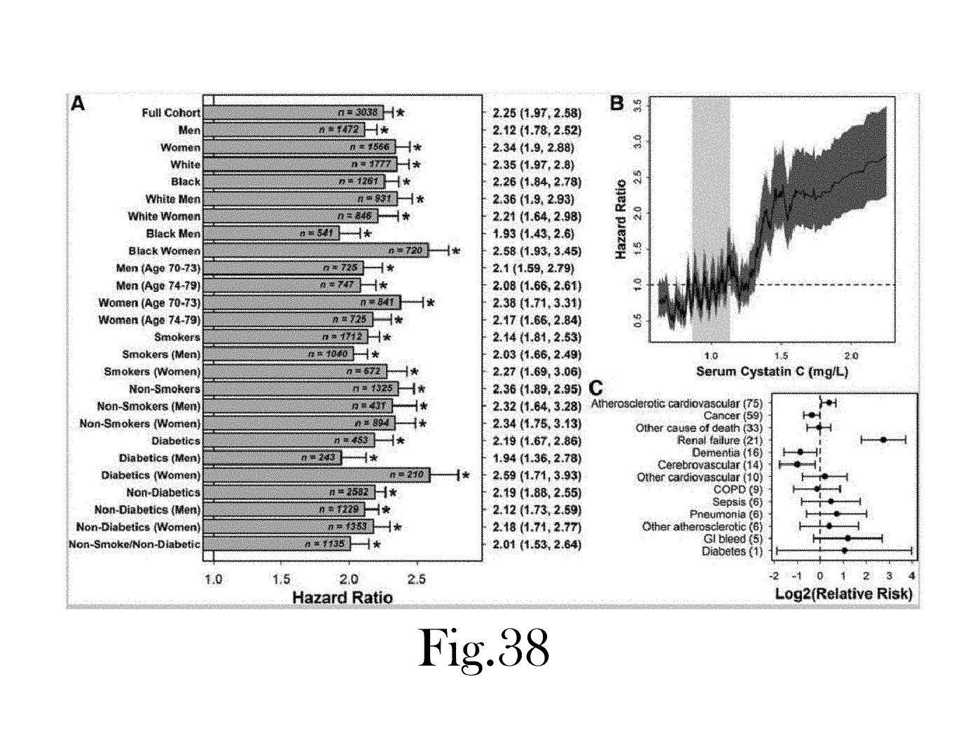

In the Health, Aging, and Body Composition Study (Health ABC) 825 people were screened for the variables that best predicted mortality over 13 years of follow-up. Mortality was most strongly associated with low Digit Symbol Substitution Test (DSST) score and elevated serum cystatin C (≥1.30 mg/mL; 12.1% of cohort; HR=2.25±0.07). These variables predicted mortality better than 823 other measures, including baseline age and a 45-variable health deficit index. Given elevated cystatin C (≥1.30 mg/mL), mortality risk was further increased by high serum creatinine, high abdominal visceral fat density, and smoking history (2.52≤HR≤3.73). Serum cystatin C warrants priority consideration for the evaluation of mortality risk in older individuals. Both variables, taken individually, predict mortality better than chronological age or a health deficit index in well-functioning older adults (ages 70-79). FIG. 38 shows the Hazard Ratios and Diseases associated with elevated Cystatin C.

Serum cystatin C levels predict mortality in the Health, Aging, and Body Composition Study (Health ABC) cohort and are associated with renal failure and atherosclerotic cardiovascular disease. (A) The hazard ratio (HR) associated with high cystatin C (cystatin C≥1.30) was estimated in the full Health ABC cohort and each of 25 subcohorts. Significant HRs are indicated by an asterisk symbol (*). Point estimates with 95% confidence intervals are listed in the right margin. Sample sizes used for each subgroup are listed at the end of each horizontal bar (participants with missing data were excluded from calculations). A 0-1 indicator was used as the independent variable in Cox regression models, where the value of the indicator was 1 for participants with high cystatin C (cystatin C≥1.30) and 0 otherwise. HR estimates are adjusted for study site (Memphis or Pittsburgh). (B) The HR associated with low to high cystatin C intervals (windows) was evaluated. Participants were sorted in ascending order according to measured cystatin C (horizontal axis). A sliding window analysis was then performed in which the HR was estimated for a window of 100 participants relative to all other participants outside of the window. The solid black line represents the estimated HR for a given window of 100 participants, and the dark grey region outlines a 95% confidence interval. The light grey vertical region in the background outlines the middle 50% of cystatin C levels among all participants (i.e., interquartile range). (C) The relative risk of (assigned) underlying causes of death was evaluated in participants with cystatin C≥1.30 (n=261 deaths) and participants with cystatin C<1.30 (n=1,083 deaths). Assigned causes of death are sorted from most frequent to least frequent among those with cystatin C≥1.30 (frequencies are given in parentheses). [Swindell, William R., et al. “Data mining identifies digit symbol substitution test score and serum cystatin C as dominant predictors of mortality in older men and women.” Rejuvenation research 15.4 (2012): 405-413.]

Reference Values

CYCTATIN C

Males:

0 days-22 years: no reference values established

23-29 years: 0.60-1.03 mg/L

30-39 years: 0.64-1.12 mg/L

40-49 years: 0.68-1.22 mg/L

50-59 years: 0.72-1.32 mg/L

60-69 years: 0.77-1.42 mg/L

70-79 years: 0.82-1.52 mg/L

>79 years: no reference values established

Females:

0 days-22 years: no reference values established

23-29 years: 0.57-0.90 mg/L

30-39 years: 0.59-0.98 mg/L

40-49 years: 0.62-1.07 mg/L

50-59 years: 0.64-1.17 mg/L

60-69 years: 0.66-1.26 mg/L

70-80 years: 0.68-1.36 mg/L

81-86 years: 0.70-1.45 mg/L

>86 years: no reference values established

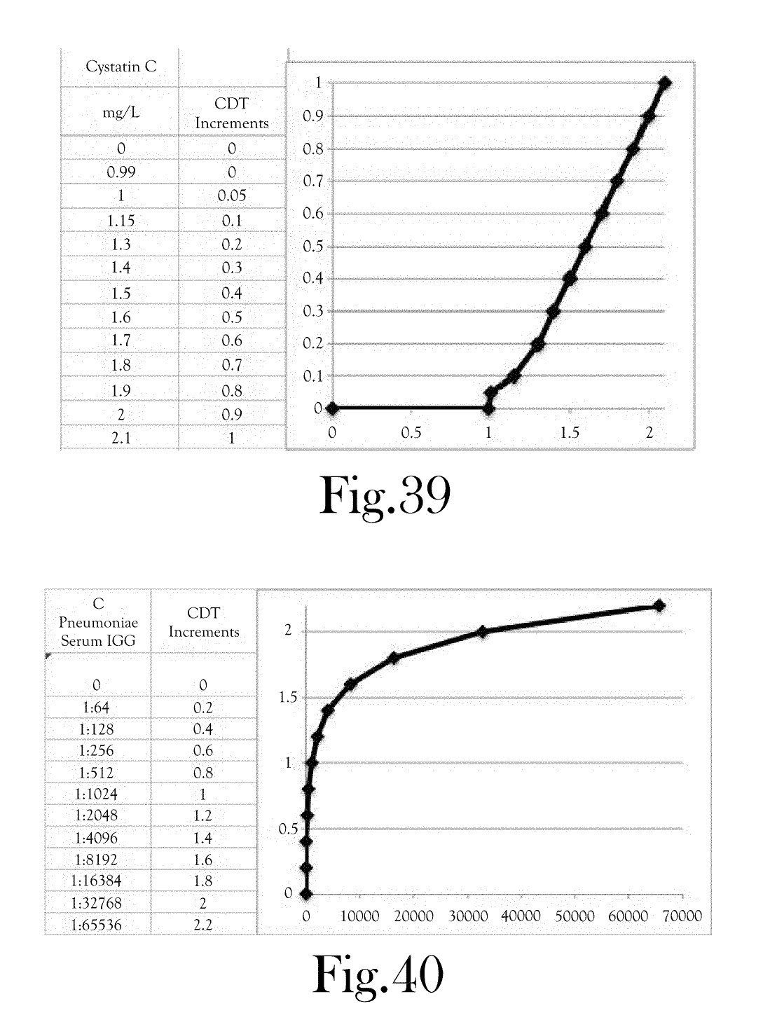

In various embodiments, cystatin C contributes to a subject's chronic disease temperature as follows: Total maximum contribution to the CDT calculation is 1.0° F. (0.56° C.). See FIG. 39.

Chlamydia (Chlamydophila) Pneumoniae (CP)

In various embodiments, chlamydia pneumoniae serves as a biomarker. Chlamydiae are obligate intracellular microorganisms which parasitize eukaryotic cells and are ubiquitous throughout the animal kingdom. Members of the chlamydial genus are considered bacteria with a unique biphasic developmental cycle having distinct morphological and functional forms. This developmental growth cycle alternates between intracellular life forms, of which two are currently recognized, a metabolically-active, replicating organism known as the reticulate body (RB) and a persistent, non-replicating organism known as the cryptic phase; and an extracellular life form that is an infectious, metabolically-inactive form known as the elementary body (EB).

EBs are small (300-400 nm) infectious, spore-like forms which are metabolically inactive, non-replicating, and found most often in the acellular milieu. EBs are resistant to a variety of physical insults such as enzyme degradation, sonication and osmotic pressure. This physical stability is thought to be a result of extensive disulfide cross-linking of the cysteine-rich major outer membrane protein (MOMP). [Bavoil et al., Infection and Immunity, 44:479-485, 1984; Hackstadt et al., Journal of Bacteriology, 161:25-31, 1985; Hatch et al., Journal of Bacteriology, 165:379-385, 1986; Peeling et al., Infection and Immunity, 57:3338-3344, 1989.] Under oxidizing conditions in the acellular milieu of the host, the outer membrane of EBs is relatively impermeable as well as resistant to inactivation. EBs are thus well suited to survive long enough outside of their hosts to be transmitted to a new host in the form of a droplet nuclei. [Theunissen et al., Applied Environmental Microbiology, 59:2589-2593, 1993,] or a fomite [Fasley et al., The Journal of Infectious Diseases, 168:493-496, 1993].

Chlamydia (more recently being classified as Chlamydophila) pneumoniae (CP) is an intracellular pathogen responsible for a number of different acute and chronic infections. It is estimated that CP may infect more than 50% of the world population, most of whom have no symptoms and may never develop symptoms assuming their immune system stays strong and is able to keep the bug at bay. The recent deepening knowledge on the biology and the use of increasingly more sensitive and specific detection measures has allowed demonstration of CP in a large number of persons suffering from different diseases including cardiovascular (atherosclerosis and stroke), central nervous system (CNS) disorders, and dementias. Infection by members of the genus Chlamydiae induces a significant inflammatory response at the cellular level. CP is the most recently classified of the genus Chlamydiae and is isolated from humans and currently is recognized as causing approximately 10 percent of community acquired cases of pneumonia. [Grayston et al., J. Inf. Dis. 161:618-625 (1990)]. This pathogen commonly infects the upper and lower respiratory tract and is now recognized as ubiquitous in humans. CP is well-accepted as a human pathogen that may be difficult to eradicate by standard antibiotic therapy. [Hammerschlag et al., Clin. Infect. Dis. 14:178-182, 1992]. CP is known to persist as a silent or mildly symptomatic pathogen, resulting in a chronic, persistent infection (J. Schacter, In: Baun A L, eg. Microbiology of Chlamydia, Boca Raton, Fla., CRC Press, 1988, pp. 153-165).

642 men (36.2%) had IgG antibodies at a titer of ≥1 in 16, of whom 362 (20.4% of all men) also had detectable IgA antibodies. There were stronger and significant relations of IgA antibodies with all-cause mortality and fatal ischemic heart disease, which persisted after adjustment for conventional cardiovascular risk factors. The odds ratios associated with detectable IgA antibodies were 1.07 (95% confidence interval 0.75 to 1.53) for all incident ischaemic heart disease, 1.83 (1.17 to 2.85) for fatal ischaemic heart disease, and 1.50 (1.10 to 2.04) for all cause mortality. [Strachan, David P., et al. “Papers Relation of Chlamydia pneumoniae serology to mortality and incidence of ischaemic heart disease over 13 years in the Caerphilly prospective heart disease study. Commentary: Chlamydia pneumoniae infection and ischaemic heart disease.” Bmj 318.7190 (1999): 1035-1040.]

C. pneumoniae infection was found to be positively associated with risk of coronary heart disease. Concentration of C. pneumoniae IgA antibody was positively associated with risk of coronary heart disease and more specifically myocardial infarction. Subjects with the highest quartile of IgA antibody showed 2.29 (95% CI, 1.21-4.33) times higher risk of coronary heart disease and 2.58 (95% CI, 1.29-5.19) times higher risk of myocardial infarction than those with lowest quartile. However, no such association was detected for IgG antibody. [Sakurai-Komada, Naomi, et al. “Association between Chlamydophila pneumoniae infection and risk of coronary heart disease for Japanese: The JPHC study.” Atherosclerosis 233.2 (2014): 338-342.]

There is powerful evidence for CP being a causal factor in some variants of the neurological illness multiple sclerosis. The presence of CP gene sequences in the cerebrospinal fluid of patients who have the disease, and culture of the organism when sensitive cultural methods are used. [Sriram S, Stratton C W, Yao S, Tharp A, Ding L, Bannan J D, Mitchell W M. Chlamydia pneumoniae infection of the central nervous system in multiple sclerosis. Ann Neurol. 1999 July; 46(1):6-14.] An association of new CP respiratory infections with episodes of clinical relapse was found. [Buljevac D, Verkooyen R P, Jacobs B C, Hop W, van der Zwaan L A, van Doorn P A, Hintzen R Q. Chlamydia pneumoniae and the risk for exacerbation in multiple sclerosis patients. Ann Neurol. 2003 December; 54(6):828-31.] A statistically significant elevation of C. pneumoniae-specific serum antibody levels when the disease shifts into the progressive form was noted [Munger K L, Peeling R W, Hernán M A, Chasan-Taber L, Olek M J, Hankinson S E, Hunter D, Ascherio A. Infection with Chlamydia pneumoniae and risk of multiple sclerosis. Epidemiology 2003 14:2 141-147]. Evidence of active C. pneumoniae protein synthesis in the central nervous system, with production of a bacterial protein evoking an antibody shown to cause death of oligodendrocyte precursor cells [Cid C, Alvarez-Cermeno J C, Camafeita E, Salinas M, Alcazar A. Antibodies reactive to heat shock protein 90 induce oligodendrocyte precursor cell death in culture. Implications for demyelination in multiple sclerosis. FASEB J. 2004 February; 18(2):409-11.] MRI improvement in antibiotic-treated patients with early disease in a small but fastidious double-blind trial of non-immunomodulatory antibiotics [Sriram S, Yao S Y, Stratton C, Moses H, Narayana P A, Wolinsky J S. Pilot study to examine the effect of antibiotic therapy on MRI outcomes in RRMS. J Neurol Sci. 2005 Jul. 15; 234(1-2):87-91.]

A study utilizing RT-PCR and ELISA techniques, demonstrate that CP infection of THP1 human monocytes promotes an innate immune response, as pro-inflammatory gene transcripts and proteins showed significant fold increases. A chronic inflammatory state is present within the AD brain and monocytes infected with CP in AD brains suggests that the pro- and chronic inflammatory states involved in AD pathogenesis arise in part by CP infection of monocytes. These data are consistent with that of previous work suggesting that amyloid could be both a response to and an initiator of inflammation in the AD brain. In effect, infection in the AD brain initiates the inflammatory cascade that results in CNS damage reflected by amyloid production/processing and deposition. [Lim, Charles, et al. “Chlamydia pneumoniae infection of monocytes in vitro stimulates innate and adaptive immune responses relevant to those in Alzheimer's disease.” Journal of neuroinflammation 11.1 (2014): 1-11.]

Reference Ranges:

C. pneumoniae IgG <1:64

C. pneumoniae IgA <1:16

C. pneumoniae IgM <1:10

Treatment: Macrolides are often the first-line treatment; tetracyclines and fluoroquinolones are also effective.

In various embodiments, chlamydia pneumoniae contributes to a subject's chronic disease temperature as follows: Total maximum contribution to the CDT calculation is 2.0° F. (1.12° C.). See FIG. 40.

Neutrophil to Lymphocyte Ratio—NLR

In various embodiments, neutrophil-to-lymphocyte ratio (NLR) is used as a biomarker. NLR, which is calculated from complete blood count with differential, is an inexpensive, easy to obtain, widely available marker of inflammation, which can aid in the risk stratification of patients with various diseases in addition to the traditionally used markers. It has been associated with arterial stiffness and high coronary calcium score, which are themselves significant markers of cardiovascular disease. NLR is reported as an independent predictor of outcome in stable coronary artery disease, as well as a predictor of short- and long-term mortality in patients with acute coronary syndromes. It is linked with increased risk of ventricular arrhythmias during percutaneous coronary intervention (PCI) and higher long-term mortality in patients undergoing PCI irrespective of indications of PCI. In patients admitted with advanced heart failure, high NLR was reported with higher inpatient mortality. Recently, NLR has been reported as a prognostic marker for outcome from coronary artery bypass grafting and postcoronary artery bypass grafting atrial fibrillation.

NLR is a marker for chronic diseases other than cardiovascular types. The following diseases and conditions are strongly associated with, predicted by, or result in worse outcomes with elevated (abnormal values of) NLR: acute coronary syndrome, acute decompensated heart failure, acute pancreatitis, acute pulmonary embolism, Alzheimer's disease, appendicitis, arterial stiffness and coronary calcium score (atherosclerosis), atrial fibrillation, bacteremia, bare-metal stent restenosis, bladder cancer, breast cancer, cardiovascular diseases (general), cervical carcinoma, chronic critical limb ischemia, colon cancer, colorectal cancer, colorectal liver metastases, coronary artery bypass grafting, coronary artery ectasia, coronary flow, epithelial ovarian cancer, esophageal cancer, essential hypertension, fibrosis, gastric cancer, general cancer patient survival, glioblastomas, hepatocellular carcinoma, large B-cell lymphoma, left ventricular function, long-term mortality, lower injection fraction, malignant mesothelioma, metabolic syndrome, myocardial infarction in type 2 diabetic patients, nasopharyngeal carcinoma, non-small cell lung cancer, ovarian cancer, pancreatic cancer, papillary microcarcinomas in thyroidal goiters, renal cell carcinoma, resected pancreatic ductal adenocarcinoma, soft-tissue sarcoma, solid tumors, steatohepatitis, stomach cancer, systemic inflammation in prevalent chronic diseases, thromboembolic stroke, ulcerative colitis, urinary protein and albumin excretion in type 2 diabetics.

In a review of NLR as an additional biomarker to be incorporated into the Framingham risk model, a study concluded that NLR fulfills the criteria to be considered as a biomarker for predicting future coronary heart disease risk in asymptomatic, apparently healthy individuals.

The predictive superiority of NLR may be due to many reasons including the fact that it is less likely to be influenced by various physiological conditions such as dehydration and exercise, even though these conditions may affect absolute number of individual cell types. Second and most importantly, NLR is a ratio of two different yet complementary immune pathways, thus integrating the deleterious effects associated with elevated neutrophils which are responsible for active nonspecific immune system activation against pathogens, neutrophilia (an indicator of inflammation) and lymphopenia (an indicator of physiological stress) that has emerged as a useful prognostic marker in many other studies where inflammation is part of the disease pathology.

NLR in Cancer:

Cancer-associated inflammation is a key determinant of outcome in patients with cancer. Various markers of inflammation have been examined over the past decade in an attempt to refine stratification of patients to treatment and predict survival. A robust marker of the systemic inflammatory response is the neutrophil-lymphocyte ratio (NLR). To date, over 60 studies (>37,000 patients) have examined the clinical utility of the NLR to predict patient outcomes in a variety of cancers. The NLR had independent prognostic value in (a) unselected cohorts (1 study of >12,000 patients), (b) operable disease (20 studies, >4000 patients), (c) patients receiving neoadjuvant treatment and resection (5 studies, >1000 patients), (d) patients receiving chemo/radiotherapy (12 studies, >2000 patients) and (e) patients with inoperable disease (6 studies, >1200 patients). These studies originated from ten different countries, in particular UK, Japan, and China. Further, correlative studies (15 studies, >8500 patients) have shown that NLR is elevated in patients with more advanced or aggressive disease evidenced by increased tumor stage, nodal stage, number of metastatic lesions and as such these patients may represent a particularly high-risk patient population. Further studies investigating the tumor and host-derived factors regulating the systemic inflammatory response, in particular the NLR, point to non-traditional treatment strategies for patients with cancer. The prognostic threshold value for NLR varied in the following manner, dependent upon the nature of the study and the exclusion/inclusion criteria of the patients: Breast>3.3, Various>5, Various>4, Colorectal>4 or >5, Gastric>3.2 or >2 or >2.2 or >3 or >2.63 or >5, esophageal>3.5 or >2.2 or >4 or >5, pancreatic>5 or >4, cholangiocarcinoma>5, liver>5 or >4, Lung>5 or >2.5 or >2.63 or >3.25 or >4.74, bladder>2.5, renal>2.7, >3, ovary>2.6, sarcoma>5, HCC>3 or >3.3 or >5, rectal>5, appendiceal>5, [Guthrie, Graeme J K, et al. “The systemic inflammation-based neutrophil-lymphocyte ratio: experience in patients with cancer.” Critical reviews in oncology/hematology 88.1 (2013): 218-230.]

In breast cancer patients, NLR is predictive of short- and long-term mortality. Patients in the highest NLR quartile (NLR>3.3) had higher 1-year (16% vs 0%) and 5-year (44% vs 13%) mortality rates compared with those in the lowest quartile (NLR<1.8) (P<0.0001). After adjusting for the factors affecting the mortality and/or NLR (using two multivariate models), NLR level >3.3 remained an independent significant predictor of mortality in both models (hazard ratio 3.13, P=0.01) (hazard ratio 4.09, P=0.002). [Azab, Basem, et al. “Usefulness of the neutrophil-to-lymphocyte ratio in predicting short-and long-term mortality in breast cancer patients.” Annals of surgical oncology 19.1 (2012): 217-224.]

The outcomes of patients with metastatic nasopharyngeal carcinoma (NPC) differ between individuals. A total of 229 patients with disseminated NPC were evaluated. The effects of pretreatment peripheral blood neutrophil, lymphocyte, and NLR on survival were examined using the proportional hazards regression model to estimate hazard ratio (HR). The relationship between short-term treatment efficacy and pretreatment NLR was analyzed using the chi-square test. The pretreatment elevated neutrophil count (p=0.020), percentage of neutrophil (p<0.001), and NLR (p=0.002) were statistically significantly associated with a poor prognosis. The cutoff value selected for NLR was 3.6. The median survival time was 15.3 months for the high-NLR group and was 23.5 months for the low-NLR group (p<0.001). [Jin, Ying, et al. “Pretreatment neutrophil-to-lymphocyte ratio as predictor of survival for patients with metastatic nasopharyngeal carcinoma.” Head & neck 37.1 (2015): 69-75.]

High neutrophil-to-lymphocyte ratio (NLR) has been reported to be a poor prognostic indicator in several solid malignancies. A systematic review of electronic databases was conducted to identify publications exploring the association of blood NLR and clinical outcome in solid tumors. Overall survival (OS) was the primary outcome, and cancer-specific survival (CSS), progression-free survival (PFS), and disease-free survival (DFS) were secondary outcomes. Data from studies reporting a hazard ratio and 95% confidence interval (CI) or a P value were pooled in a meta-analysis. Pooled hazard ratios were computed and weighted using generic inverse-variance and random-effect modeling. All statistical tests were two-sided. One hundred studies comprising 40559 patients were included in the analysis, 57 of them published in 2012 or later. Median cutoff for NLR was 4. Overall, NLR greater than the cutoff was associated with a hazard ratio for OS of 1.81 (95% CI=1.67 to 1.97; P<0.001), an effect observed in all disease subgroups, sites, and stages. Hazard ratios for NLR greater than the cutoff for CSS, PFS, and DFS were 1.61, 1.63, and 2.27, respectively (all P<0.001). [Templeton, Arnoud J., et al. “Prognostic role of neutrophil-to-lymphocyte ratio in solid tumors: a systematic review and meta-analysis.” Journal of the National Cancer Institute 106.6 (2014): dju124.]

The NLR has prognostic value in patients with glioblastoma. A prospective study on patients receiving surgery for glioblastoma. The mean NLR ratio was 6.7±4.6. Using receiver operating characteristic curve analysis, an NLR cut-off value of 4.7 was determined to best predict survival. Patients with NLR ratios exceeding 4.7 differed significantly from those with NLR ratios ≤4.7 and were associated with reduced survival. Patients with gross total tumor excision had a median survival of 18 months, whereas the median survival time was 11 months in patients with subtotal tumor excision. No significant difference in survival was observed with respect to patient age, gender, Karnofsky performance status, or tumor location. Using multivariate analysis, NLR and extent of tumor resection were identified as factors with independent prognostic power. NLR is a biomarker of glioblastoma aggressiveness. [Alexiou, George A., et al. “Prognostic significance of neutrophil-to-lymphocyte ratio in glioblastoma.” Neuroimmunology and Neuroinflammation 1.3 (2014): 131.]

Preoperative NLR, in combination with CA125, is a method of identifying ovarian cancers, and an elevated NLR may predict an adverse outcome in ovarian cancer. Preoperative NLR in ovarian cancer subjects (mean 6.02) was significantly higher than that in benign ovarian tumor subjects (mean 2.57), benign gynecologic disease subjects (mean 2.55), and healthy controls (mean 1.98) (P<0.001). The sensitivity and specificity of NLR in detecting ovarian cancer was 66.1% (95% CI, 59.52-72.68%) and 82.7% (95% CI, 79.02-86.38%), respectively (cutoff value: 2.60). In early stage ovarian cancer, CA125 was not elevated in 19 out of 49 patients. Seven (36.8%) of these 19 patients were NLR positive [Cho, HanByoul, et al. “Pre-treatment neutrophil to lymphocyte ratio is elevated in epithelial ovarian cancer and predicts survival after treatment.” Cancer Immunology, Immunotherapy 58.1 (2009): 15-23.]

NLR in Cardiovascular Diseases:

Total WBC count is confirmed to be an independent predictor of death and heart attack in patients with or at high risk for coronary artery disease (CAD), but greater predictive ability is provided by high neutrophils alone or low Lymphocyte counts. The greatest risk prediction is given by the NRL, with Quintile 4 versus Quintile 1 (>4.71 versus <1.96) increasing the hazard 2.2-fold. FIG. 19 shows how natural logarithmic transformation was found to normalize the distributions. [Horne, Benjamin D., et al. “Which white blood cell subtypes predict increased cardiovascular risk?.” Journal of the American College of Cardiology 45.10 (2005): 1638-1643.] FIG. 41 shows the white blood cell subtype and cardiovascular hazard ratio.

Cardiovascular events risk was evaluated in the context of traditional Framingham risk score (FRS) model. Analysis of National Health and Nutrition Examination Survey-III (1998-94) including subjects aged 30-79 years free from CHD or CHD equivalent at baseline. Primary endpoint was death from ischemic heart disease. NLR was divided into four categories: <1.5, ≥1.5 to <3.0, 3.0-4.5 and >4.5. Statistical analyses involved multivariate Cox proportional hazards models as well as discrimination, calibration and reclassification. 7363 subjects were included with a mean follow up of 14.1 years. There were 231 (3.1%) CHD deaths, more in those with NLR >4.5 (11%) compared to NLR <1.5 (2.4%), p<0.001. Adjusted hazard ratio of NLR >4.5 was 2.68 (95% CI 1.07-6.72, p=0.035). Thus NLR can independently predict CHD mortality in an asymptomatic general population cohort. It reclassifies intermediate risk category of FRS, with significant upward reclassification. [Shah, Neeraj, et al. “Neutrophil lymphocyte ratio significantly improves the Framingham risk score in prediction of coronary heart disease mortality: insights from the National Health and Nutrition Examination Survey-III.” International journal of cardiology 171.3 (2014): 390-397.]

A higher NLR was independently associated with arterial stiffness and coronary calcium score (CCS). The ORs (95% CIs) for a high brachial-ankle pulse wave velocity by NLR quartiles were 1.00, 0.76 (0.41-1.39), 1.08 (0.61-1.90), and 2.12 (1.18-3.83) after adjusting for confounding variables. [Park, Byoung-Jin, et al. “Relationship of neutrophil-lymphocyte ratio with arterial stiffness and coronary calcium score.” Clinica Chimica Acta 412.11 (2011): 925-929.]

Alzheimer's Disease:

Alzheimer's (AD) risk and prognosis is predicted by the blood neutrophil-lymphocyte ratio (NLR). 241 AD patients and 175 patients with normal cognitive function were evaluated. The mean±SD NLR of AD patients was significantly higher than that of patients with normal cognitive function (3.21±1.35 vs. 2.07±0.74, p<0.001, respectively). Receiver operating characteristic curve analysis suggested that the optimum NLR cutoff point for AD was 2.48 with 69.29% sensitivity, 79.43% specificity, 82.30% positive predictive values and 65.30% negative predictive values. Logistic regression analysis showed that elevated NLR (OR: 4.774, 95% CI: 2.821-8.076, p<0.001) was an independent variable for predicting AD. [Kuyumcu, Mehmet Emin, et al. “The evaluation of neutrophil-lymphocyte ratio in Alzheimer's disease.” Dementia and geriatric cognitive disorders 34.2 (2012): 69-74.]

Appendicitis:

The total white cell count is not consistently a reliable predictor of appendicitis. It has been reported that the lymphocyte count can fall in acute appendicitis. A retrospective study of patients undergoing appendectomy for suspected appendicitis over a 2-year period identified 402 patients. Histopathology confirmed appendicitis in 367 (91%). A total of 298 (79%) patients with appendicitis had an elevated preoperative total white cell count. The neutrophil:lymphocyte ratio was calculated for each patient. Using an upper limit of 3.5:1, it was found that 324 (88%) of patients with appendicitis had a ratio equal to or greater than this value. [Goodman, David A., Chantelle B. Goodman, and John S. Monk. “Use of the neutrophil: lymphocyte ratio in the diagnosis of appendicitis.” The American surgeon 61.3 (1995): 257-259.]

Metabolic Syndrome:

Seventy patients with metabolic syndrome (MS) and 71 age- and sex-matched control participants were included. Patients were classified into 3 groups based on the number of MS criteria: group 1 (with 3 criteria), group 2 (with 4 criteria), and group 3 (with 5 criteria). The NLR was calculated from complete blood count. Patients with MS had significantly higher NLR compared to the control group. Moreover, the group 3 patients had higher NLR than those in groups 2 and 1 (P=0.008 and P=0.078, respectively). NLR increased as the severity of MS increased (r=0.586, P<0.001). The cutoff level for NLR with optimal sensitivity and specificity was calculated as 1.84. Serum glucose and high-sensitive C-reactive protein level were found to be independent predictors of an NLR value greater than 1.84.

Neutrophils to Lymphocytes Reference Ranges:

Neutrophils—2.0-7.0×10^{c}/l (40-80%)

Lymphocytes—1.0-3.0×10⁹/l (20-40%)

Although normal NLR reference ranges are not established, a normal value may be obtained by determining the ratio between normal values for neutrophils and lymphocytes.

In exemplary embodiments, a neutrophil-to-lymphocyte ratio (NLR) of 1.5 may be considered the upper limit for good health.

In various embodiments, NLR contributes to a subject's chronic disease temperature as follows:

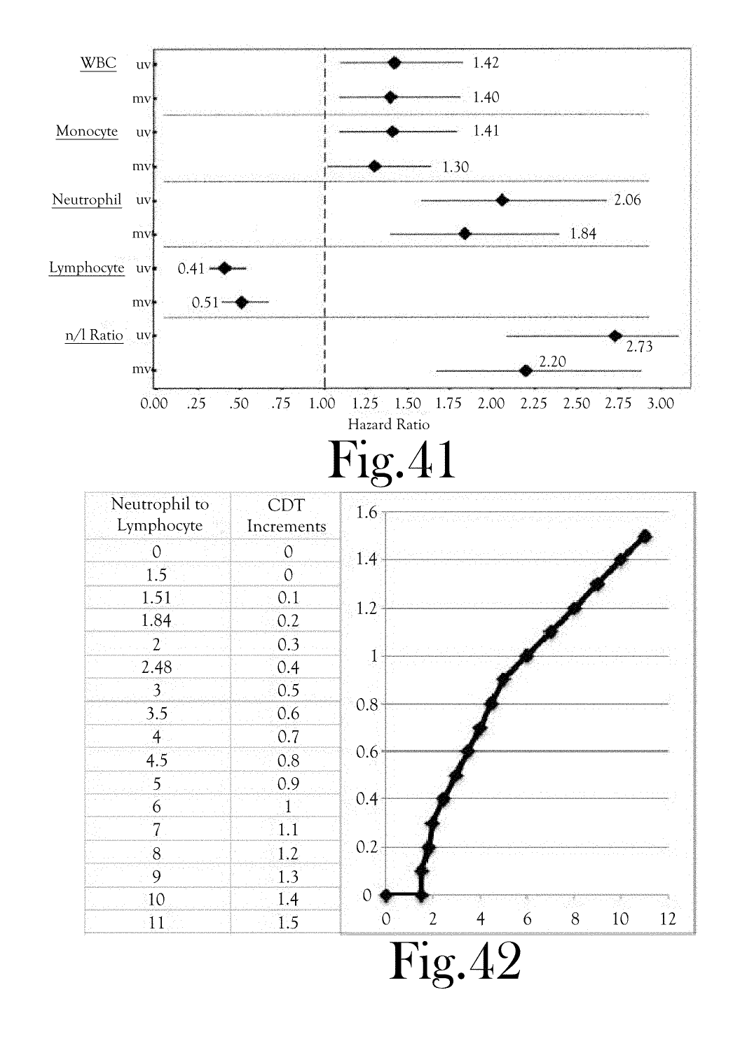

Total maximum contribution to the CDT calculation is 1.5° F. (0.84° C.). See FIG. 42.

Neutrophil Counts

Neutrophilic granulocytes (neutrophils), the most abundant but also very short-lived human white blood cells, act as first defenders against infections. Neutrophil turnover is rapid, ˜109 cells per kilogram of body weight leave the bone marrow per day in healthy humans (2, 3).

Neutrophils are the major leukocytes in the peripheral blood. The white blood cell (WBC) count normally drawn from a patient is made up of a number of different leukocytes which include neutrophils at 60-70%, lymphocytes at 28%, monocytes at 5%, eosinophils at 2-4%, and basophils at 0.5% of the total. When a WBC count is done on a patient, the lab value reflects the leukocytes distributed within the blood and not those in the bone marrow, tissue or attached to the endovascular lining of blood vessels. It is evident that the neutrophils make up the greatest amount of leukocytes in the total WBC count and thus can have the greatest impact on changes in the WBC count.

Neutrophils are also called polymorphonuclear leukocytes (PMN) because of the number of stages they go through in their appearance. They are initially released from the bone marrow as immature neutrophils that are characterized as having a nonsegmented, band like appearing nucleus. As such these immature neutrophils are called “bands”. An increase in the number of these immature neutrophils in circulation can be indicative of a bacterial infection for which they are being called to fight against. This is normally seen or called a “left shift” in a WBC differential. As the immature neutrophils become activated or exposed to bacterial pathogens, their nucleus will take on a segmented appearance. These and other neutrophils can be found in several compartments within the body, but the two compartments of importance are the marginal compartment (those neutrophils attached to the endothelium of the blood vessel) and the circulating compartment (those circulating in the blood vessels along with other cells).

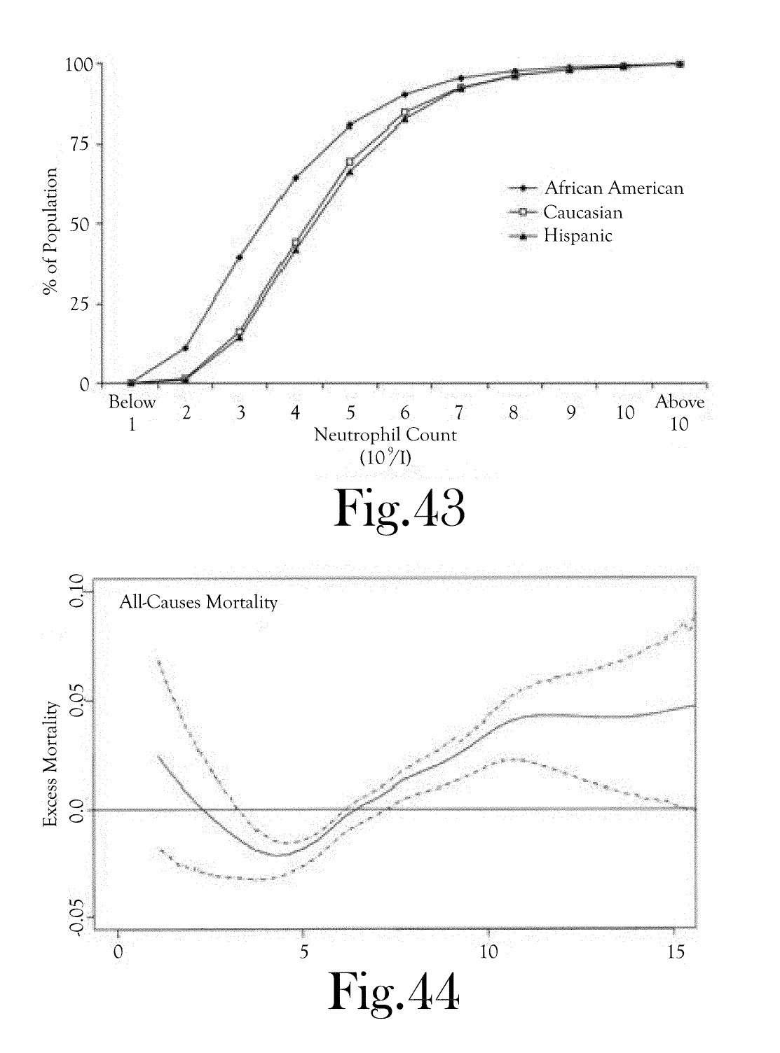

Baseline neutrophil counts are relatively stable in individuals but have a considerable normal range in healthy humans. A survey of more than 25,000 Americans found a mean neutrophil count of 4.3×10⁹/l in adult males and 4.5×10⁹/l in females for Caucasian participants. Environmental factors contribute to a global decrease of neutrophil counts in an US-American population from 1958 to 2002. In addition, the genetic or epigenetic background is important. Mean neutrophil counts are lower in African Americans: in one study, 3.5×10⁹/l in males and 3.8×10⁹/l in females. “Benign ethnic neutropenia” is a condition found in up to 5% of African Americans and is defined as a neutrophil count <1.5×10⁹/l without apparent overt cause or complication. [Ruggiero, Carmelinda, et al. “White blood cell count and mortality in the Baltimore Longitudinal Study of Aging.” Journal of the American College of Cardiology 49.18 (2007): 1841-1850.] FIG. 43 shows the normal levels for neutrophil counts in presumed healthy subjects.

Neutrophilia (neutrophil counts elevated above normal levels) is a classical indicator of acute inflammation of infectious or multiple other causes such as acute arteriosclerotic events or trauma, whereas idiopathic and acquired (e.g., drug-induced) forms of neutropenia predispose to infections. However, total white blood cell counts (WBCs), which are mainly determined by neutrophil counts in healthy humans, are also relevant in the absence of acute events. Increased WBCs have long been associated with increased all-cause mortality. A prospective study conducted over 44 years revealed a J-shaped association curve of neutrophil, but not lymphocyte, count and all-cause mortality. Increased WBCs have long been associated with increased all-cause mortality. [von Vietinghoff, Sibylle, and Klaus Ley. “Homeostatic regulation of blood neutrophil counts.” The Journal of Immunology 181.8 (2008): 5183-5188.] FIG. 44 shows a J-shaped association between neutrophil counts and mortality.

Neutrophils are the first defense against invading microorganisms. Increased susceptibility to common pathogens has usually been attributed to extremely low counts (<0.5×10⁹/l), and individuals with “low normal” counts or ethnic neutropenia have not been reported to be at increased risk as long as counts are not further decreased. However, the probability of contracting tuberculosis from patients with open pulmonary disease was inversely correlated with baseline neutrophil counts. In contrast, an increased total WBC and neutrophil count has been shown to be an independent risk factor for cardiovascular mortality in a number of studies and subsequent metaanalyses. Various clinical trials have reported an association between increased neutrophil count in peripheral blood and short-term post-MI adverse outcomes and worse angiographic findings. [von Vietinghoff, Sibylle, and Klaus Ley. “Homeostatic regulation of blood neutrophil counts.” The Journal of Immunology 181.8 (2008): 5183-5188.]

In a study of mortality and neutrophil counts, at a 7.8 year follow up of 3316 patients scheduled for coronary angiography, 745 died, of which 484 died from cardiovascular events. After entering conventional risk factors and removing patients with a current infection, neutrophil count (HR [95% CI]=1.90 [1.39, 2.60], P<0.001) and the neutrophil/lymphocyte ratio (HR [95% CI]=1.68 [1.24, 2.27], P=0.003) emerged as independent predictors of cardiovascular mortality. After mutual adjustment, neutrophil count (HR [95% CI]=1.87 [1.35, 2.50], P<0.001) out-performed C-reactive protein (HR [95% CI] 1.32 [0.99, 1.78], P=0.06) as a predictor of cardiovascular mortality. [Hartaigh, Briain, et al. “Which leukocyte subsets predict cardiovascular mortality? From the LUdwigshafen RIsk and Cardiovascular Health (LURIC) Study.” Atherosclerosis 224.1 (2012): 161-169.]

In a study of neutrophil count, cancer incidence and cancer mortality, a neutrophil count range of 1.0-5.7 revealed neutrophil count was associated with a significant but non-linear increase in cancer mortality in the highest tertile compared to the lowest. [Davidovics, Sarah A., et al. “Neutrophil count, cancer incidence and cancer mortality: disparate relationships by race.” Cancer Research 73.8 Supplement (2013): 2525-2525.]

In a study of myocardial infarction, non-surviving patients, mostly female, had significantly higher absolute neutrophil counts. Multivariate analysis revealed neutrophil count as an independent predictor of mortality [OR=2.94, CI (1.03-8.44), P=0.04]. Subgroups analysis of WBC by ROC-analysis was performed to determine the sensitivity and specificity of factors in predicting in-hospital mortality. The cutoff point of neutrophil >9.68-×1000 cells/mm³ had a sensitivity of 60% and specificity of 66.2% in predicting post-MI mortality. Increased neutrophil count was associated with higher in-hospital mortality, post-infarction pump failure and occurrence of serious ventricular arrhythmias within the first 24 hours. The presence of neutrophilia after ST elevation myocardial infarction (higher than the cutoff value of 9.68×1000 cells/mm³) was predictive of pump failure and significant increase in the frequency of ventricular arrhythmias within the first post MI day. [Ghaffari, Samad, et al. “The predictive value of total neutrophil count and neutrophil/lymphocyte ratio in predicting in-hospital mortality and complications after STEMI.” Journal of cardiovascular and thoracic research 6.1 (2014): 35.]

Reference Range

Differential blood count gives relative percentage of each type of white blood cell and also helps reveal abnormal white blood cell populations (eg, blasts, immature granulocytes, or circulating lymphoma cells in the peripheral blood).

Absolute neutrophil count (ANC) is the real number of white blood cells that are neutrophils. The absolute neutrophil count is commonly called the ANC. The ANC is not measured directly. It is derived by multiplying the WBC count times the percent of neutrophils in the differential WBC count. The percent of neutrophils consists of the segmented (fully mature) neutrophils)+the bands (almost mature neutrophils). The normal range for the ANC=1,500 to 8,000/mm³ with other published normal ranges being 2,000 to 7,000/mm³ and 3,000 to 7,500/mm³. The normal percentage of neutrophils as part of the total WBC is reported to be 54-75%; 50-60%; and 40-60%. High percentages of neutrophils of the total WBC, regardless of the WBC total level is indicative of underlying disease but is not considered here, as part of the chronic disease temperature assessment.

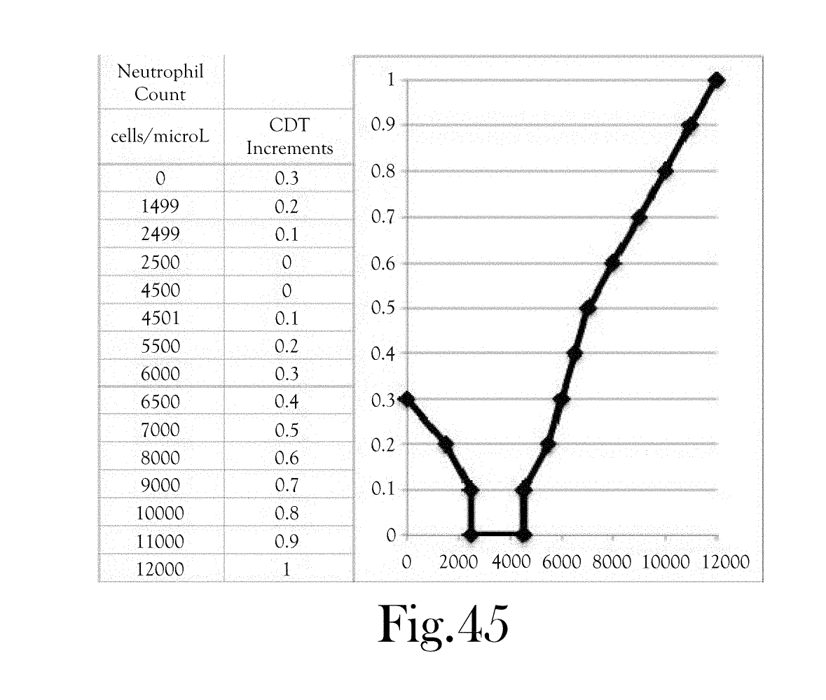

In various embodiments, neutrophil counts contribute to a subject's chronic disease temperature as follows: Total maximum contribution to the CDT calculation is 1.0° F. (0.56° C.). See FIG. 45.

Cataract

In various embodiments cataract is used as a biomarker. The transparency of the eye lens depends on maintaining the native tertiary structures and solubility of the lens crystallin proteins over a lifetime. Cataract, the leading cause of blindness worldwide, is caused by protein aggregation (misfolded or unfolded protein response) within the protected lens environment. With age, covalent protein damage accumulates through pathways thought to include UV radiation, oxidation, deamidation, inflammation, and truncations. Experiments suggest that the resulting protein destabilization leads to partially unfolded, aggregation-prone intermediates and the formation of insoluble, light-scattering protein aggregates. These aggregates either include or overwhelm the protein chaperone content of the lens.

Proteopathy refers to a class of diseases in which certain proteins become structurally abnormal, and thereby disrupt the function of cells, tissues and organs of the body. Often the proteins fail to fold into their normal configuration; in this misfolded state, the proteins can become toxic in some way (a gain of toxic function) or they can lose their normal function. The proteopathies (also known as proteinopathies, protein conformational disorders, or protein misfolding diseases) include such diseases as Creutzfeldt—Jakob disease, Alzheimer's disease, Parkinson's disease, prion disease, amyloidosis, and a wide range of other disorders including cataract. Thus signs of proteopathy is a sign of disease and disease progression.

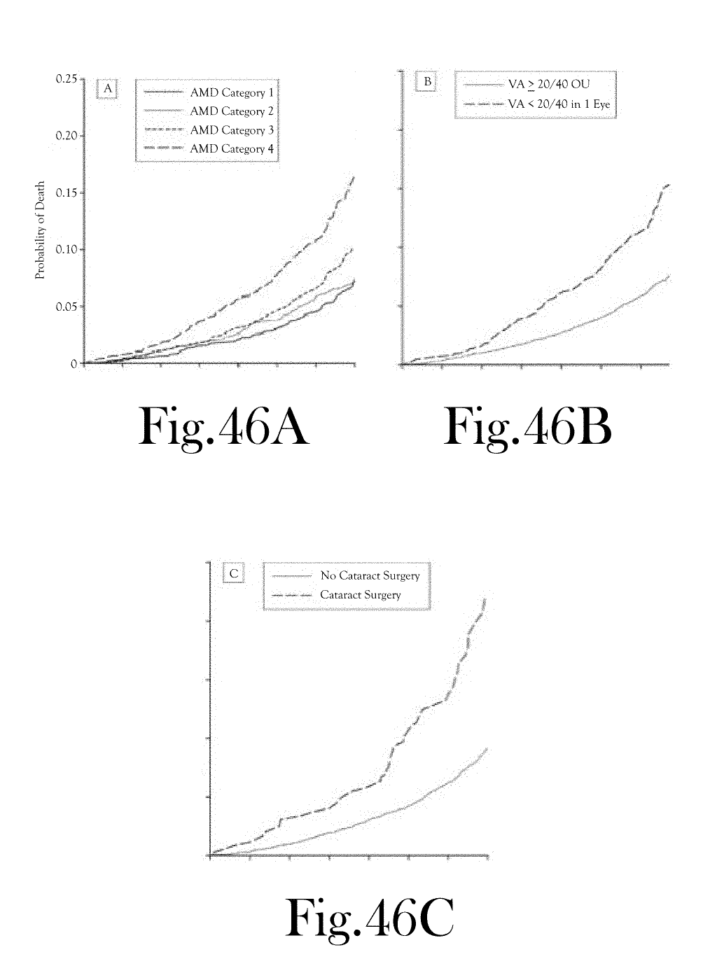

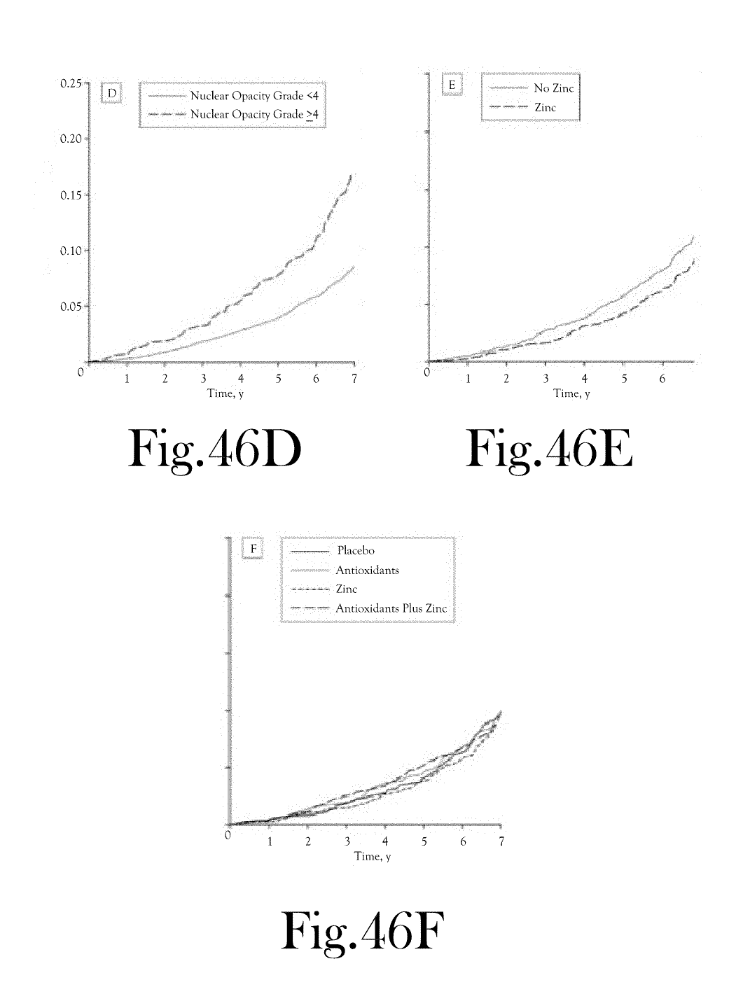

The NIH sponsored a formal trial on eye diseases in the 1990s. That trial was called the AREDS, short for the Age-Related Eye Disease Study. [Age-Related Eye Disease Study Research Group. “The Age-Related Eye Disease Study (AREDS) system for classifying cataracts from photographs: AREDS report no. 4.” American journal of ophthalmology 131.2 (2001): 167-175.] The goal of the Age-Related Eye Disease Study was to learn about macular degeneration and cataract, two leading causes of vision loss in older adults. The study looked at how these two diseases progress and what their causes may be. The AREDS study involved 11 medical centers with more than 4,700 people enrolled across the country. The study was supported by the National Eye Institute, part of the Federal government's National Institutes of Health. An unexpected result came out of AREDS. Certain eye diseases are predictors of premature or early death (mortality). In other words, what this study revealed is that a rapidly aging eye occurs in a rapidly (accelerated) aging body. Nuclear opacity and cataract surgery were associated with increased all-cause mortality and cancer deaths. The decreased survival of AREDS participants with AMD and cataract suggests these conditions may reflect systemic processes rather than only localized disease. [Grigorian, Adriana Paula. “Associations of Mortality With Ocular Disorders and An Intervention of High-Dose Antioxidants and Zinc in the Age-Related Eye Disease Study.” Evidence-Based Ophthalmology 5.4 (2004): 230-231.] FIG. 22 below shows the AREDS study data. FIGS. 46A-F show the Age-Related Eye Disease Study Illustrating the Probability of Death Associated with Eye Diseases.

Many studies show the cataract/mortality association.

The Priverno Eye Study. This was a population-based cohort study of incidence of blindness, low vision, and survival. Lens opacities are associated with a higher risk of death. The purpose of this study was to further investigate the relationships between different types of lens opacity and patient survival. The analysis of the Priverno data confirms an association between lower survival and cataracts, particularly those confined to the lens nucleus and those that had already prompted surgery.

The Barbados Eye Study. The purpose of this study was to determine incidence and risk factors for each main cause of visual loss in an African-Caribbean population. Incidence of visual impairment was high and significantly affected quality of life. Age-related cataract and open angle glaucoma caused ˜75% of blindness, indicating the need for early detection and treatment. The connection between metabolic and cardiovascular disease and ocular indications and diseases is strong in this study.

The Blue Mountain Eye Study. This was the first large population-based assessment of visual impairment and common eye diseases of a representative older Australian community sample. The findings demonstrate the connection between eye and systemic diseases. In particular, cardiovascular risk factors were prominent for eye diseases including: Cataract, macular degeneration, Glaucoma, and retinopathy.

The Beijing Eye Study. This study was a population-based study that included 4439 subjects who were initially examined in 2001 through blood tests and ocular assessment. The data suggest that glaucoma, particularly angle-closure glaucoma, may be associated with an increased rate of mortality in adult Chinese in Greater Beijing.

The Rotterdam Eye study. This study started in 1990 in a suburb of Rotterdam, among 10,994, men and women aged 55 and over. Major risk factors that were found for macular degeneration included atherosclerosis (cardiovascular disease). Retinal venular (microvessel) diameters play a role in predicting cardiovascular disorders. Dilated retinal venules at baseline were predictive for stroke, cerebral infarction, dementia, white brain matter lesions, impaired glucose tolerance, diabetes mellitus and mortality. Inflammation is part of these diseases. The Rotterdam Study concluded that both ARM and cataract are predictors of shorter survival because they have risk factors that also affect mortality.

Numerous lines of evidence suggest common factors linking AD-associated pathology in the brain and lens. Comparing aged controls with AD patients, researchers observed amyloid-β (Aβ) deposits exclusively in AD lenses in the cytoplasm of deep cortical lens fiber cells. [Goldstein L E, Muffat J A, Cherny R A, Moir R D, Ericsson M H, et al. (2003) Cytosolic beta-amyloid deposition and supranuclear cataracts in lenses from people with Alzheimer's disease. Lancet 361: 1258-1265. ]A subsequent study demonstrated increased deposition of Aβ in lens and distinctive deep cortical localization in persons with Down Syndrome, a common chromosomal disorder that is invariantly associated with early-onset age-dependent AD neuropathology resulting from APP gene triplication and Aβ overexpression. Supranuclear and deep cortical cataract has been documented in transgenic mice expressing human Aβ and fiber cell membrane defects similar to those described in human cataracts have been observed in transgenic mice carrying a complete copy of human APP from the Down Syndrome critical region of chromosome 21. In addition, AD-linked Aβ accumulation and light-scattering cytosolic Aβ microaggregate formation co-localize with amyloid pathology and subequatorial supranuclear and deep cortical fibers of human subjects with late-onset AD and Down syndrome associated AD. [Jun, Gyungah, et al. “delta-Catenin is genetically and biologically associated with cortical cataract and future Alzheimer-related structural and functional brain changes.” PLoS One 7.9 (2012): e43728.]

The Salisbury Eye Evaluation Project consisted of a random sample of 2520 residents of Salisbury, Md, aged 65 to 84 years. At baseline, lens photographs were taken to document nuclear, cortical, posterior subcapsular cataract, and mixed opacities. Data on education, smoking, alcohol use, hypertension, diabetes and other comorbid conditions, handgrip strength, and body mass index were also collected. Two-year follow-up was conducted for mortality and cause of death. Nuclear opacity, particularly severe nuclear opacity, and mixed opacities with nuclear were significant predictors of mortality independent of body mass index, comorbid conditions, smoking, age, race, and sex (mixed nuclear: odds ratio, 2.23; 95% confidence interval, 1.26-3.95). Lens opacity status is an independent predictor of 2-year mortality, an association that could not be explained by potential confounders, Table 20.

TABLE 20
Association for Cataract Opacity Types and 2-Year Mortality

| | Odds Ratio (95% |
|---|---|
| Lens Opacity Type | Confidence Interval) |
| Severe nuclear ≥3 only | 1.27 (0.76-2.15) |
| Mixed opacity (with nuclear) | 2.23 (1.26-3.95) |
| Mixed opacity (without nuclear) | 0.86 (0.35-2.09) |
| Posterior subcapsular cataract only | 0.77 (0.10-5.89) |

Causes of death were broadly grouped into cardiovascular, cancer, and miscellaneous for cause specific analyses. Most deaths were from cardiovascular disease (41%), with cancer causing 33% of deaths. In models predicting cause-specific mortality, mixed nuclear opacities were significantly associated with cancer deaths, Table 21.

TABLE 21
Association of Mixed Nuclear Opacity
and Cause-Specific Mortality

| | % of Deaths | Odds Ratio (95% |
|---|---|---|
| Cause of Death | Overall | Confidence Interval) |
| Cancer | 33 | 2.85 (1.14-7.01) |
| Cardiovascular | 41 | 1.78 (0.76-4.14) |
| Other | 26 | 2.39 (0.78-7.38) |

Results of analyses that focused on cause-specific mortality suggest a more than 2-fold risk associated with mixed nuclear opacity for cancer and, similarly for cardiovascular and other causes of death. [West, Sheila K., et al. “Mixed Lens Opacities and Subsequent Mortality.” Arch Ophthalmol 118 (2000): 393-397.]

Cataract Grading: A cataract is any opacity of the lens, whether it is a small local opacity or a diffuse general loss of transparency. To be clinically significant the cataract must cause a significant reduction in visual acuity or a functional impairment. The three common types of cataract are nuclear, cortical, and posterior subcapsular. A cataract-free lens is one in which the nucleus, cortex, and subcapsular areas are free of opacities; the subcapsular and cortical zones are free of dots, flecks, vacuoles, and water clefts; and the nucleus is transparent, although the embryonal nucleus may be visible.

Cataracts may be graded by visual inspection and assignment of numerical values to indicate severity. Alternative grading systems advocated for use in epidemiological studies of cataract are the Oxford Clinical Cataract Classification and Grading System, 17 the Johns Hopkins system, [West S K, Rosenthal F, Newland H S, Taylor H R. Use of photographic techniques to grade nuclear cataracts. Invest Ophthalmol Vis Sci 1988; 29:73.] and the Lens Opacity Classification System (LOCS, LOCS II, and LOCS III). [Chylack L T. Instructions for applying the lens opacity classification systems (LOCS) in grading human cataractous changes at the slit lamp. Center for Clinical Cataract Research. Boston: 1987:1-7.] Photographs of slit lamp cross-sections of the lens are used as references for grading nuclear opalescence and nuclear color, and photographs of the lens seen by retroillumination are used as references for grading cortical and posterior subcapsular cataract.

In most clinical settings, reference photographs are not available. Therefore, a less-sensitive four-point grading system modified from LOCS II21 is commonly used. Despite its limitations, this simple 1, 2, 3, 4 grading scale can be used to record the extent of nuclear, cortical, and posterior subcapsular lenticular opacity changes and a guide for this clinical form of cataract grading is shown in Table 22. [Care of the Patient with Cataract: Reference Guide for Clinicians. American Optometric Association, 1995.]

TABLE 22
Cataract Grading Scale

| Cataract | | | | |
|---|---|---|---|---|
| Type | Grade 1 | Grade 2 | Grade 3 | Grade 4 |
| Nuclear | Mild | Moderate | Pronounced | Severe |
| Yellowing and | | | | |
| sclerosis of the | | | | |
| lens nucleus | | | | |
| Cortical | Obscures | Obscures | Obscures | Obscures |
| Measured as | 10% of intra- | 10%-50% of | 50%-90% of | more than |
| aggregate | pupillary | intra- | intra- | 90% of intra- |
| percentage of | space | pupillary | pupillary | pupillary |
| the | | space | space | space |
| intrapupillary | | | | |
| space occupied | | | | |
| by the opacity | | | | |
| Posterior | Obscures 3% | Obscures | Obscures | Obscures |
| subcapsular | of the area of | 30% of the | 50% of the | more than |
| Measured as | the posterior | area of the | area of the | 50% of the |
| aggregate | capsule | posterior | posterior | area of the |
| percentage of | | capsule | capsule | posterior |
| the posterior | | | | capsule |
| capsular area | | | | |
| occupied by | | | | |
| the opacity | | | | |

Nuclear sclerosis (NS) may be graded by evaluating the average color and opalescence of the nucleus as a continuum from grade 1 (mild or early) to grade 4+ (severe advanced milky or brunescent NS). Cortical cataract (CC) and subcapsular opacities should be visualized as “aggregate” and quantified on the basis of the percentage of intrapupillary space obscured. Posterior subcapsular cataract (PSC) is graded on the basis of percentage of the area of the posterior capsule obscured. A PSC in the line of sight may be much more debilitating and the description of grading should reflect this (e.g., grade 2+ PSC in line of sight).

In various embodiments, cataract(s) contributes to a subject's chronic disease temperature as follows:

| | Contribution to chronic |
|---|---|
| Nuclear Cataract | disease temperature (F.) |
| None | 0.00 |
| Grade 1 (Mild) - per eye | 0.05 |
| Grade 2 (Moderate) - per eye | 0.10 |
| Grade 3 (Pronounced) - per eye | 0.20 |
| Grade 4 (Severe) - per eye | 0.30 |

| | Contribution to chronic |
|---|---|
| Cortical Cataract | disease temperature (F.) |
| None | 0.00 |
| Grade 1 (Mild) - per eye | 0.025 |
| Grade 2 (Moderate) - per eye | 0.05 |
| Grade 3 (Pronounced) - per eye | 0.10 |
| Grade 4 (Severe) - per eye | 0.15 |

| | Contribution to chronic |
|---|---|
| Posterior Subcapsular Cataract | disease temperature (F.) |
| None | 0.00 |
| Grade 1 (Mild) - per eye | 0.025 |
| Grade 2 (Moderate) - per eye | 0.05 |
| Grade 3 (Pronounced) - per eye | 0.10 |
| Grade 4 (Severe) - per eye | 0.15 |

Total maximum contribution to the CDT calculation is 1.0° F. (0.56° C.).

Macular Degeneration

In various embodiments, macular degeneration is used as a biomarker. Age-related macular degeneration (AMD) is a progressive, chronic disease of the central retina, and is a leading cause of blindness and low vision among older adults. AMD has both early and late stages. Early AMD is usually not associated with loss of vision. Vision loss in late AMD is caused either by neovascular disease, with growth of new blood vessels that leak and scar underneath the central retina, or by geographic atrophy in which an area of the retina in the macula atrophies. Neovascular or wet AMD is responsible for most AMD-related severe visual loss. The most important risk factor for any stage of AMD is old age. Pooled data from seven population-based studies showed that the prevalence of geographic atrophy in the United States was 0.3% in 60-64 year olds, 0.5% in 65-69 year olds, 0.9% in 70-74 year olds, 1.8% in 75-79 year olds, and 6.9% in those 80 or older. The respective rates for neovascular disease were 0.4%, 0.6%, 1.2%, 2.2%, and 8.2%.

Several studies have attempted to establish whether persons with AMD are at increased risk of death, particularly resulting from vascular causes, but results have been equivocal. This inconsistency in findings is speculated to be due to AMD being associated with other systemic conditions that are risk factors for mortality, so that controlling for these risk factors nullified the association between AMD and mortality in some but not all studies. [Gopinath, Bamini, et al. “Age-related macular degeneration and risk of total and cause-specific mortality over 15 years.” Maturitas (2015).] Differences in study design, age-sex population distribution, and follow-up duration could also explain these differences. The Study of Osteoporotic Fractures has looked at the relationship between AMD and mortality risk over 15 years. This study showed that women aged 80+ years with any AMD had increased risk of death from any cause or cardiovascular disease (CVD). [Coleman, Anne L., et al. “Impact of age-related macular degeneration on vision-specific quality of life: Follow-up from the 10-year and 15-year visits of the Study of Osteoporotic Fractures.” American journal of ophthalmology 150.5 (2010): 683-691.]

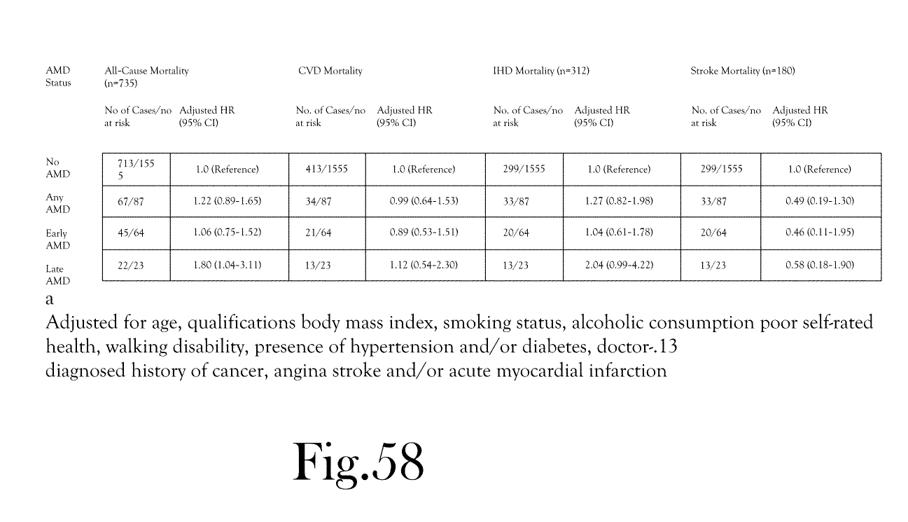

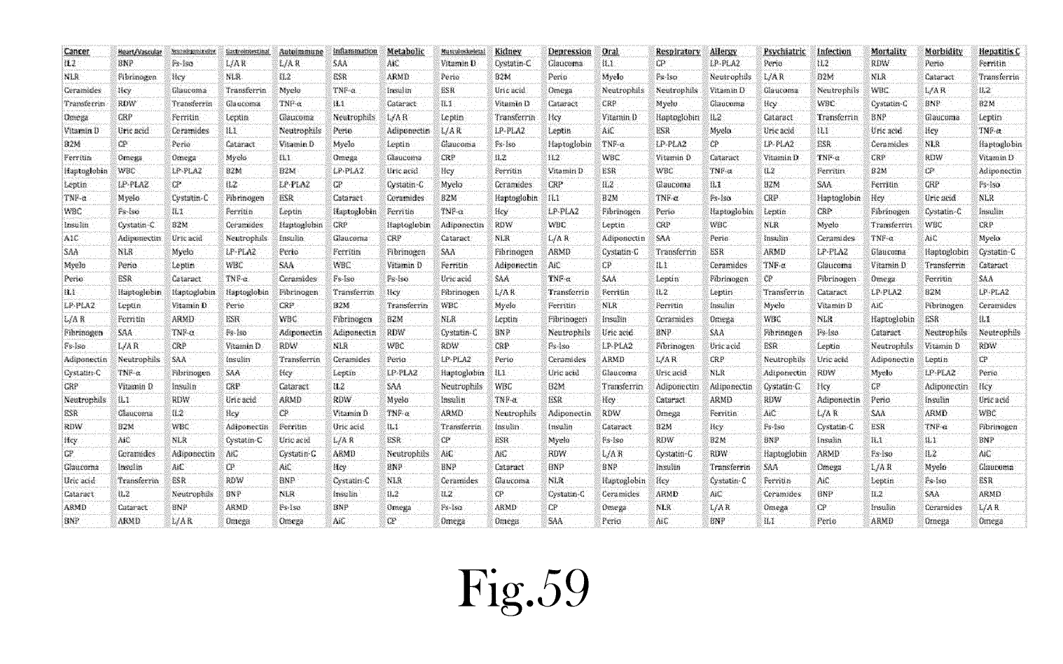

AMD (any, early or late) was assessed for an association with all-cause and cause-specific mortality (CVD; ischemic heart disease, IHD; and stroke mortality) 15 years later, independent of the effects of various potential confounders (e.g., age, sex, smoking, body mass index, diabetes, hypertension, cancer, angina, myocardial infarction, walking disability and self-rated health). This cohort study illustrates that late AMD is a significant and independent predictor of 15-year all-cause mortality in men, and stroke mortality in women. Men or women with early AMD were not at a higher risk of dying compared to persons without AMD. These epidemiological data add to the existing evidence-base that late AMD is a marker of biological aging and poorer survival in older adults, as shown in FIG. 58.

In the AREDS Study, during median follow-up of 6.5 years, 534 (11%) of 4753 AREDS participants died. In fully adjusted models, participants with advanced age-related macular degeneration (AMD) compared with participants with few, if any, drusen had increased mortality (relative risk [RR], 1.41; 95% confidence interval [CI], 1.08-1.86). Advanced AMD was associated with cardiovascular deaths.

Thirteen cohort studies (8 prospective and 5 retrospective studies) with a total of 1,593,390 participants with 155,500 CVD events (92,039 stroke and 62,737 CHD) were included in a meta-analysis. Among all studies, early AMD was associated with a 15% (95% CI, 1.08-1.22) increased risk of total CVD. The relative risk was similar but not significant for late AMD (RR, 1.17; 95% CI, 0.98-1.40). In analyses restricted to the subset of prospective studies, the risk associated with early AMD did not appreciably change; however, there was a marked 66% (95% CI, 1.31-2.10) increased risk of CVD among those with late AMD. [Wu, Juan, et al. “Age-related macular degeneration and the incidence of cardiovascular disease: a systematic review and meta-analysis.” PloS one 9.3 (2014): e89600.]

Macular degeneration is a potential biomarker for Alzheimer's disease. A conclusion from the Rotterdam Study suggests that the neuronal degeneration occurring in age-related maculopathy and Alzheimer's disease may, to some extent, have a common pathogenesis. [Klaver, Caroline C W, et al. “Is age-related maculopathy associated with Alzheimer's disease: The Rotterdam Study.” American journal of epidemiology 150.9 (1999): 963-968.] A supporting conclusion was reached in a study that review 197 separate publications. Specifically, Alzheimer's disease and macular degeneration have, for the most part, a common disease mechanism. Age-related macular degeneration (AMD) is a late-onset, neurodegenerative retinal disease that shares several clinical and pathological features with Alzheimer's disease (AD), including stress stimuli such as oxidative stress and inflammation. In both diseases, the detrimental intra- and extracellular deposits have many similarities. Aging, hypertension, obesity, arteriosclerosis, and smoking are risk factors to develop AMD and AD. Cellular aging processes have similar organelle and signaling association in the retina and brain tissues. [Kaarniranta, Kai, et al. “Age-related macular degeneration (AMD): Alzheimer's disease in the eye.” Journal of Alzheimer's Disease 24.4 (2011): 615-631.]

AMD Grading: Rates of progression from early to advanced AMD is assigned based on the presence or absence in each eye of 2 easily identified retinal abnormalities, drusen and pigment abnormalities. Large drusen and any pigment changes were particularly predictive of developing advanced AMD. The scoring system assigns to each eye 1 risk factor for the presence of 1 or more large (≥125 μm, width of a large vein at disc margin) drusen and 1 risk factor for the presence of any pigment abnormality. Risk factors are summed across both eyes, yielding a 5-step scale (0-4) on which the approximate 5-year risk of developing advanced AMD in at least one eye increases in this easily remembered sequence: 0 factors, 0.5%; 1 factor, 3%; 2 factors, 12%; 3 factors, 25%; and 4 factors, 50%. For persons with no large drusen, presence of intermediate drusen in both eyes is counted as 1 risk factor.

In various embodiments, macular degeneration contributes to a subject's chronic disease temperature as follows:

Total maximum contribution to the CDT calculation is 1.0° F. (0.56° C.).

| | Contribution to chronic |
|---|---|
| Macular degeneration | disease temperature (F.) |
| 0 risk factor | 0.00 |
| 1 risk factor | 0.10 |
| 2 risk factors | 0.20 |
| 3 risk factors | 0.30 |
| 4 risk factors | 0.40 |
| Wet (bleeding) AMD - 1 eye | 0.50 |
| Wet (bleeding) AMD - 1 eye and 1 | 0.60 |
| risk factor in the “dry” eye | |
| Wet (bleeding) AMD - 1 eye and 2 | 0.70 |
| risk factors in the “dry” eye | |
| Wet (bleeding) AMD - both eyes | 1.00 |

Glaucoma

In various embodiment, glaucoma is used as a biomarker. Glaucoma is a common eye disease that can cause blindness if left undiagnosed and untreated. Glaucoma is a leading cause of blindness in the United States and other industrialized countries. In most cases, the symptoms of early-stage glaucoma are minimal or nonexistent. There are several different types of glaucoma, and they have been classically divided into the categories of primary or secondary open-angle or angle-closure glaucoma. Glaucoma, or glaucomatous optic neuropathy, is characterized by a chronic, slowly progressive loss of retinal ganglion cells and their neurons. The disease is associated with remodeling of the optic nerve head and the retina leading to the major clinical signs: characteristic optic nerve head cupping and visual field defects. Elevated intraocular pressure (IOP) is one of the major risk factors for developing glaucoma. By far the most common reason for an increased IOP is the reduced outflow capacity of aqueous humor, usually located at the anterior chamber angle and trabecular meshwork. When the chamber angle is normally developed and not blocked by the iris and there is no other apparent cause for an increased IOP, then the term primary open-angle glaucoma (POAG) is used. However, a number of conditions show that increased IOP does not necessarily lead to glaucoma and that glaucoma can develop even under normal IOP. Other risk factors may be involved as well. Some of these additional risk factors can be found in the eye, such as a thin cornea or disk hemorrhages, whereas other factors are systemic.

Despite intense research, the pathogenesis of primary open-angle glaucoma (POAG) is still not completely understood. There is ample evidence for a pathophysiological role of elevated intraocular pressure; however, several systemic factors may influence onset and progression of the disease. Systemic peculiarities found in POAG include alterations of the cardiovascular system, autonomic nervous system, immune system, as well as endocrinological, psychological, and sleep disturbances. An association between POAG and other neurodegenerative diseases, such as Alzheimer disease and Parkinson disease, has also been described.

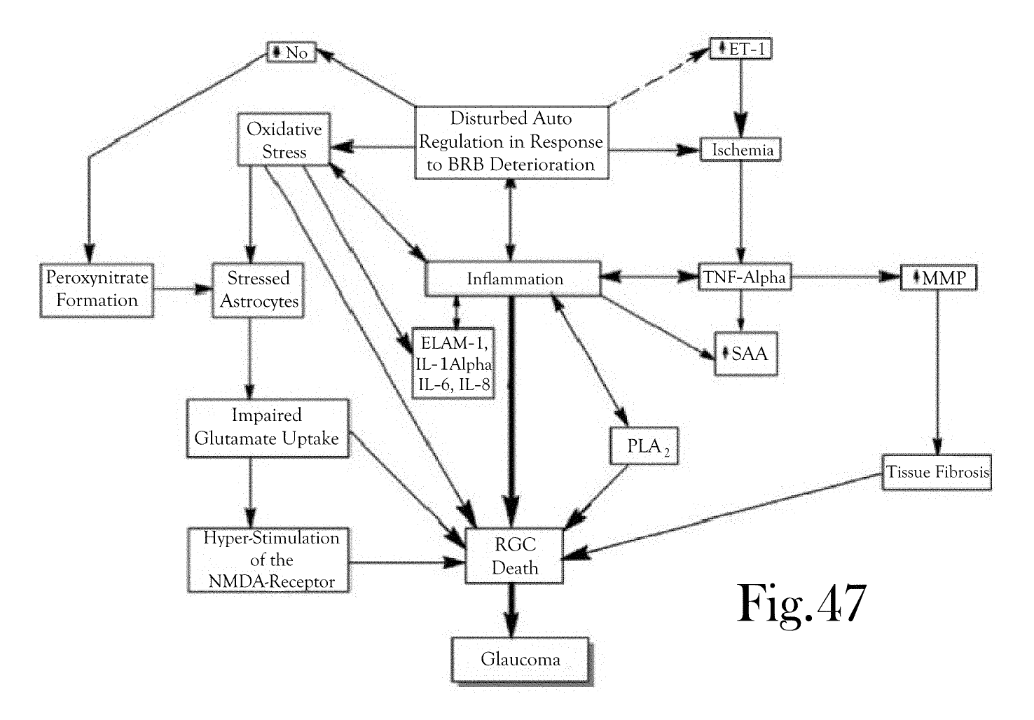

In patients with POAG, both systemic arteriosclerosis and sclerotic changes in the ocular vessels and in the internal carotid artery have been observed. Most of the studies undertaken thus far in this field find a certain relevance of altered systemic blood pressure in glaucoma. Mounting evidence suggests a true association between POAG and alterations of the immune system. Models of retinal ganglion cell death in POAG have revealed that inflammatory components may directly link increased IOP and ischemia with retinal ganglion cell loss. Generally, inflammation occurs in response to ischemic injury, with an acute and prolonged inflammatory process characterized by production of pro=inflammatory mediators and infiltration of various types of inflammatory cells into the ischemic tissue through the intercellular space between vascular endothelial cells. The blood=brain barrier around the optic nerve head has been shown to leak in glaucomatous eyes. The probably relationship of impaired blood brain barrier and the pathogenesis of glaucoma suggests that inflammatory responses may participate in the fate of the retinal ganglion cells by inducing pro-apoptotic cascade reactions in the retinal ganglion cells, FIG. 23. [Vohra, Rupali, James C. Tsai, and Miriam Kolko. “The role of inflammation in the pathogenesis of glaucoma.” Survey of ophthalmology 58.4 (2013): 311-320.] In FIG. 47 a flowchart is shown summarizing the role of Inflammation in the Pathogenesis of Glaucoma.

There is increasing evidence that glaucomatous damage extends from retinal ganglion cells to the lateral geniculate nucleus and to the visual cortex in the brain. Recent studies also indicate a possible relationship between Alzheimer disease and glaucoma. A study of all death certificates of the United States from 1978, found a high frequency of glaucoma in senile and presenile dementia. Axonal and retinal ganglion cell degeneration in the optic nerves was found in 8 of 10 patients with Alzheimer disease. In 10 patients with Alzheimer disease loss was predominant in the largest class of retinal ganglion cells (M cells), with a dropout of retinal ganglion cells ranging from 30% to 60%.286. In a retrospective analysis, pattern-electroretinography were recorded for 42 patients with glaucoma, 13 patients with Alzheimer disease, 58 patients with diabetes mellitus, and 92 control subjects. The pattern-electroretinography showed a similarity of the changes between the Alzheimer disease and glaucoma subjects. [Pache, Mona, and Josef Flammer. “A sick eye in a sick body? Systemic findings in patients with primary open-angle glaucoma.” Survey of ophthalmology 51.3 (2006): 179-212.]

Deaths including glaucoma, as either an underlying cause or a contributing cause of death, were selected from US multiple-cause-of-death data for the years 1990 to 2003 and combined with population data from the US Census Bureau to calculate mortality rates. Logistic regression was used to determine whether reporting of accidents and/or selected systemic disorders are associated with glaucoma on the death certificate. Fifteen thousand two hundred twenty-eight glaucoma-related deaths (0.05%) were identified during the years under study. Black males had the highest glaucoma-related mortality rate with 9.4 deaths per 1,000,000 persons annually, whereas Hispanic females had the lowest mortality rate at 1.8 deaths per 1,000,000. After adjusting for age, sex, and race/ethnicity, positive associations were found between glaucoma and hypertension [Odds ratio (OR): 4.89; 95% confidence interval (CI)=4.73-5.05], diabetes (OR: 2.60; 95% CI=2.50-2.71), asthma (OR: 3.14; 95% CI=2.72-3.62), and accidents of all types (OR: 1.45; 95% CI=1.35-1.55). Glaucoma is an important contributor to mortality for certain individuals. The disparities in mortality rates observed among race/ethnic strata may be attributed to differences in access to care as well as true differences in disease incidence and/or severity among racial groups. [Bennion, Jonathan R., et al. “Analysis of glaucoma-related mortality in the United States using death certificate data.” Journal of glaucoma 17.6 (2008): 474-479.]

Every available treatment to prevent progressive glaucomatous optic neuropathy has potential adverse effects and involves a certain amount of risk and financial expense. Conventional first-line treatment of glaucoma usually begins with the use of a topical selective or nonselective β-blocker or a topical prostaglandin analog. Second-line drugs of choice include α-agonists and topical carbonic anhydrase inhibitors. Parasympathomimetic agents, most commonly pilocarpine, are considered third-line treatment options. For patients who do not respond to antiglaucoma medications, laser trabeculoplasty and incisional surgery are further methods that can be used to lower intraocular pressure. The results of clinical trials have reaffirmed the utility of antiglaucoma medications in slowing the progression of the disease.

In various embodiments, glaucoma contributes to a subject's chronic disease temperature as follows:

| | Contribution to chronic |
|---|---|
| Glaucoma | disease temperature (F.) |
| No glaucoma | 0.00 |
| Preglaucoma, unspecified - 1 eye | 0.10 |
| Borderline glaucoma (glaucoma | 0.20 |
| suspect) - 1 eye | |
| Open-angle glaucoma - 1 eye | 0.35 |
| Sum the values for each eye to | |
| determine the total chronic disease | |
| temperature contribution from glaucoma. | |

Total maximum contribution to the CDT calculation is 0.7° F. (0.39° C.).

Biomarker Panels and Calculations of the Chronic/Specific Disease Temperature™

Any combination of the biomarkers described herein can be used to assemble a biomarker panel, which is detected or measured as described herein, to determine the chronic/specific disease temperature of a human. As is generally understood in the art, a combination may refer to an entire set or any subset or subcombination thereof. The term “biomarker panel,” “biomarker profile,” or “biomarker fingerprint” refers to a set of biomarkers. As used herein, these terms can also refer to any form of the biomarker that is measured. While individual biomarkers are useful as diagnostics, it has been found that a combination of biomarkers can provide greater value in determining a particular health or disease status than single biomarkers alone. Specifically, the detection of a plurality of biomarkers in a sample can increase the sensitivity and/or specificity of the test. Thus, in various embodiments, a biomarker panel may include 1, 2, 3, 4, 5, 6, 7, 8, 9, 10 or more types of biomarkers. In various exemplary embodiments, the biomarker panel consists of a minimum number of biomarkers to generate a maximum amount of information. Thus, in various embodiments, the biomarker panel consists of 1, 2, 3, 4, 5, 6, 7, 8, 9, 10 or more types of biomarkers.

The present invention provides a biomarker panel comprising or consisting of any combination of the biomarkers outlined herein. Any number of biomarkers may be used to determine a subject's chronic disease temperature. Assigned to each biomarker is a risk score expressed in temperature units (Fahrenheit). The base temperature indicating that the biomarker is in a normal healthy range for a subject is 0.00 degrees Fahrenheit. The upper temperature of the biomarker indicating that the subject has or is at risk for chronic disease varies based on the diagnostic power of the biomarker. Some biomarkers have upper temperature risk scores of less than 1.00 degree Fahrenheit while other biomarkers have upper temperature risk score of 1.00 degrees Fahrenheit or greater depending upon their predictive power for current or future chronic disease. The Fahrenheit temperature value associated with each value of a biomarker is added to the normal healthy temperature, assigned as 98.6 degrees Fahrenheit. For each biomarker, the actual determined (measured) value is converted to a chronic/specific disease temperature increment contribution value. Each biomarker chronic/specific disease temperature value is added to 98.6 to arrive at the subjects initial estimated chronic/specific disease temperature. A maximum value for the chronic/specific disease temperature is 98.6+9.00=107.6. The value 9.00F is derived from the upper temperature measurement of 107.6F minus the normal temperature measurement of 98.6F. The maximum chronic/specific disease temperature contribution for a given biomarker is found in each biomarker chronic disease temperature increment contribution table. Three scenarios lead to the calculation of a final estimated chronic disease temperature.

Scenario 1. The sum of all biomarkers maximum chronic disease temperature contributions <9.00F. When the sum of the maximum chronic disease temperature contribution values for all biomarkers used to determine a subjects actual chronic disease temperature is less than 9.00F, then the calculated chronic disease temperature is an underestimate of the subjects actual chronic disease temperature. Such chronic disease temperatures are expressed with a “≥” preceding the chronic disease temperature value. The value is still used as a risk determinant for the patient but those with elevated chronic disease temperatures are encouraged to undergo more testing.

Scenario 2. The sum of all biomarker maximum chronic disease temperature contributions >9° F. When the sum of the maximum chronic disease temperature contribution values for all biomarkers used to determine a subjects actual chronic disease temperature is greater than 9° F., then the actual chronic disease temperature is calculated using the following formula:

Chronic disease temperature™=[98.6 F+(sum of biomarker chronic disease temperature values)(9.00F/sum of maximum chronic disease temperature values)]. While 9° F. is used in this scenario, a different value, such as 7° F. could be used.

Scenario 3. The sum of all biomarker maximum chronic disease temperature contributions=9 F. The actual chronic disease temperature is 98.6 F plus the sum of the actual chronic disease temperature contribution by each biomarker tested.

While 9° F. is the selected value of degrees is used in these scenarios, a different selected value of degrees, such as 7° F. could be used.

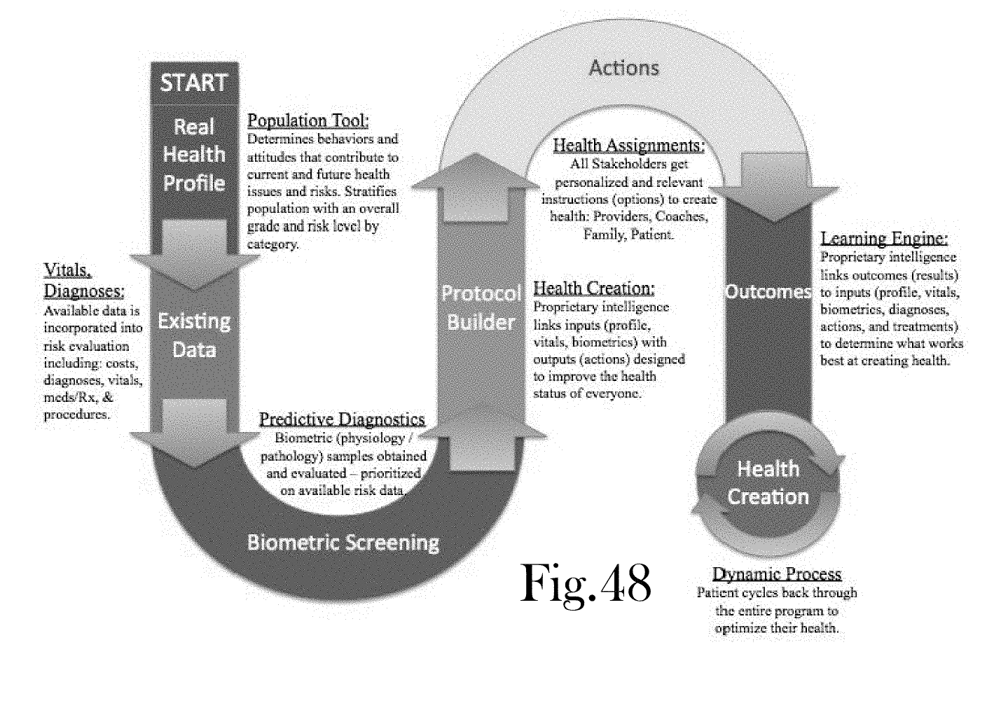

FIG. 48 shows a high level representation of one embodiment of the invention.

### EXAMPLE 1

Patient A obtains a blood test for white blood cell counts. Result: 9,600 cells/microliter. The contribution to the Patient A's chronic disease temperature by the WBC level of 9,600 is 0.60 degrees Fahrenheit. Since no other biomarker was obtained, and the maximum contribution from the WBC biomarker is 1.50 degrees Fahrenheit, which is less than 9.00F, the patient's chronic disease temperature is, at the time of the test, 98.6+0.60≥99.2 degrees Fahrenheit.

### EXAMPLE 2

Patient B obtains a blood test for WBC, homocysteine, C-reactive protein; vitamin D, fibrinogen, myeloperoxidase, and red blood cell distribution width. In addition, the patient undergoes an eye pathology evaluation for cataract and macular degeneration. The results with contribution to chronic disease temperature in parenthesis: WBC=9,200 cells/microliter (0.60); homocysteine=21 micromoles/liter (1.1); C-reactive protein=7.5 mg/liter (0.70); vitamin D=19 ng/ml (0.50); fibrinogen=425 mg/dl (0.60); myeloperoxidase=460 pmol/liter (0.40); red blood cell distribution width=14.4% (0.40); cataract=nuclear Grade 3, two eyes (0.40) and posterior subcapsular Grade 3, two eyes (0.10) (total contribution 0.50); macular degeneration=wet bleeding AMD−1 eye (0.50) and 2 risk factors, fellow eye (0.20) (total contribution 0.70). The total potential contribution from all biomarkers used to calculate the chronic disease temperature=6.00 degrees Fahrenheit which yields a maximum chronic disease temperature of 104.60 degrees Fahrenheit. The chronic disease temperature for patient B=98.6+0.6+1.1+0.7+0.5+0.6+0.4+0.4+0.4+0.1+0.5+0.2=104.1 degrees Fahrenheit. The total maximum temperature contribution for the biomarkers evaluated for Patient B=11.10 degrees Fahrenheit. The upper temperature range for the chronic disease temperature scale is 107.60 degrees Fahrenheit reflecting an 9.00 degree Fahrenheit range. The temperature for Patient B is an overestimate of their chronic disease temperature because of the number of biomarkers used in the determination. The actual estimated chronic disease temperature is 98.6+[(5.5)×(9.00/11.10)]=103.1 (rounded to the first tenth decimal place).

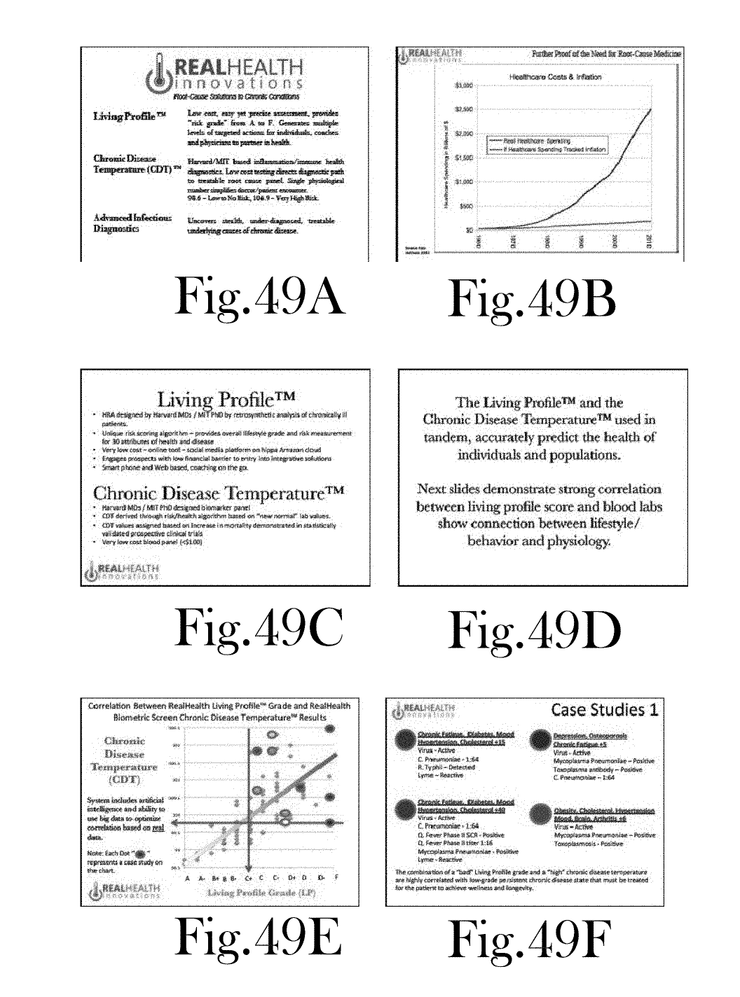

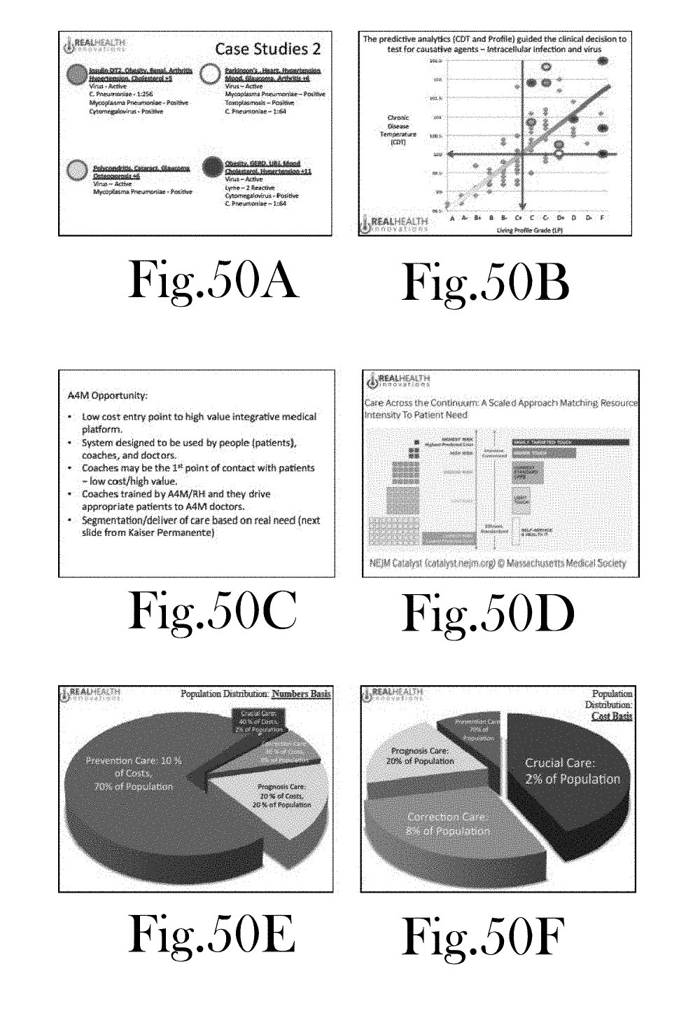

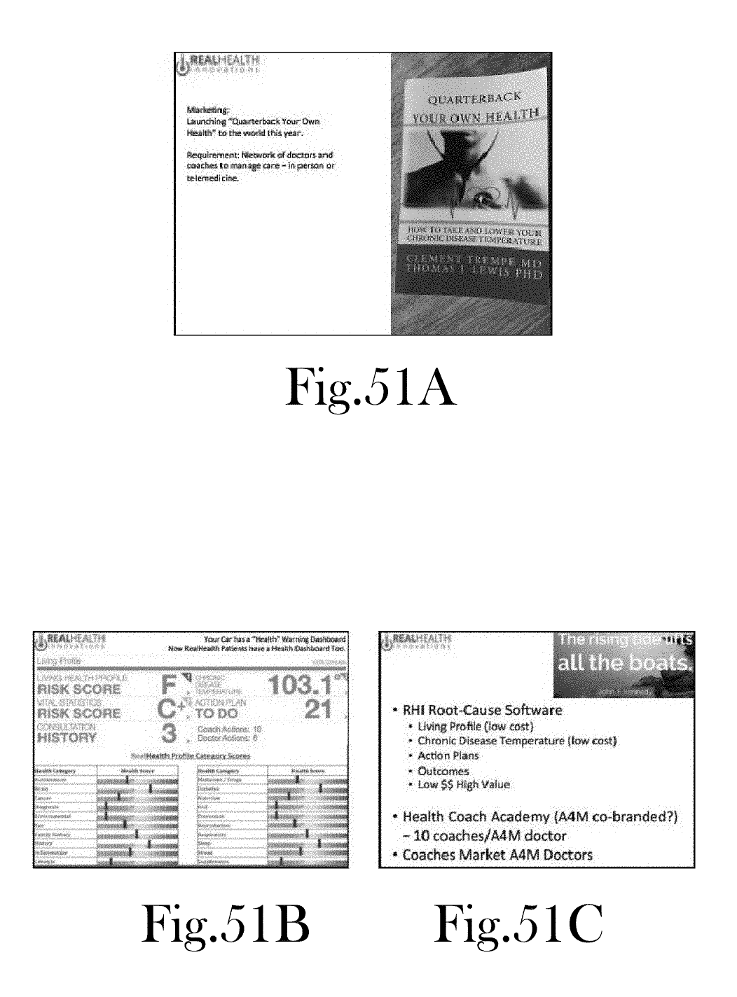

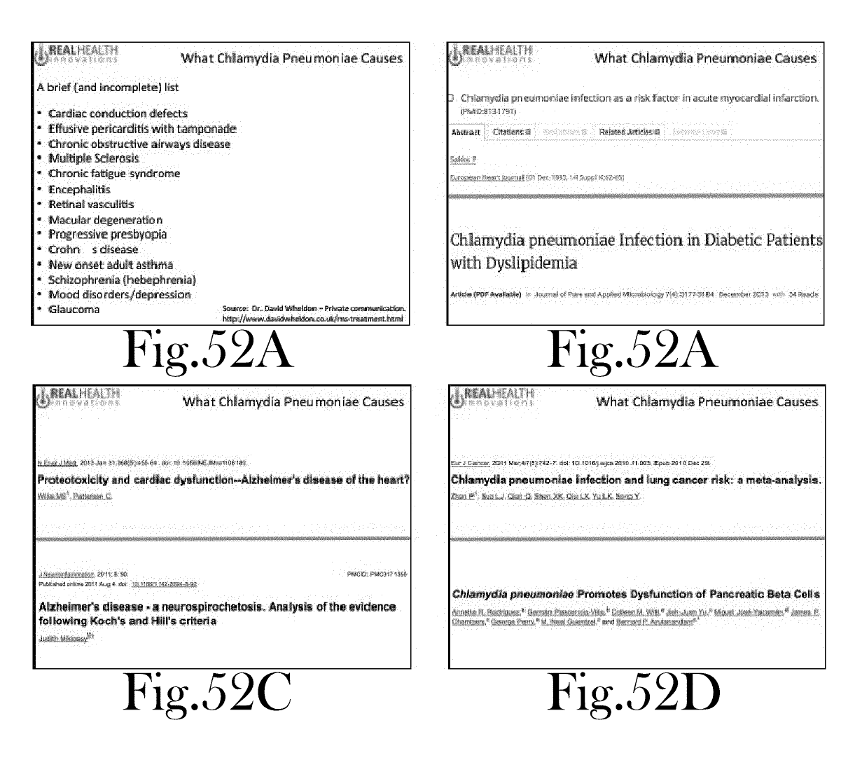

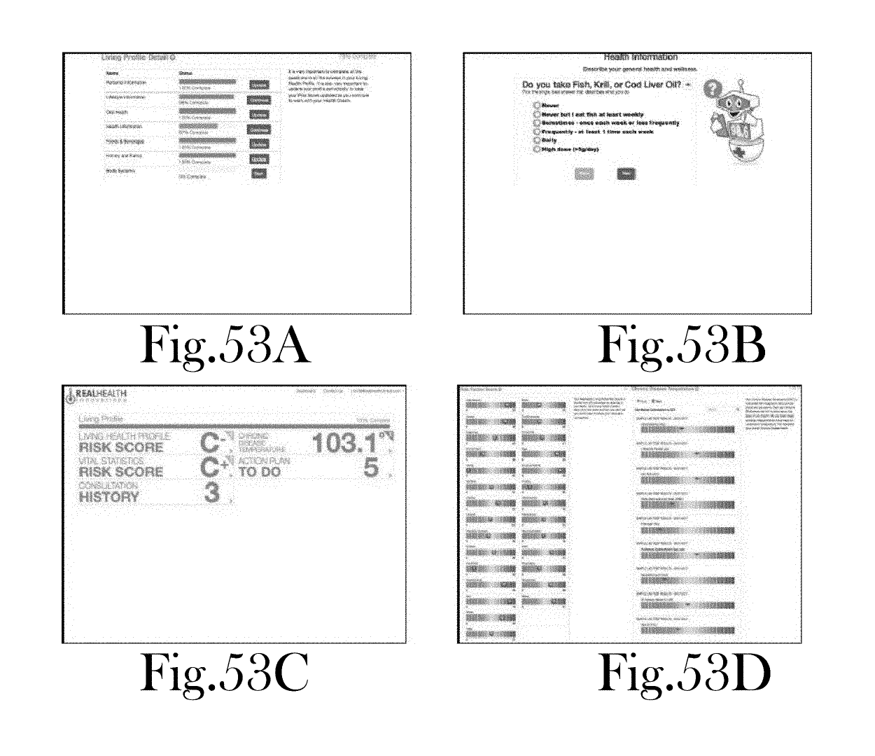

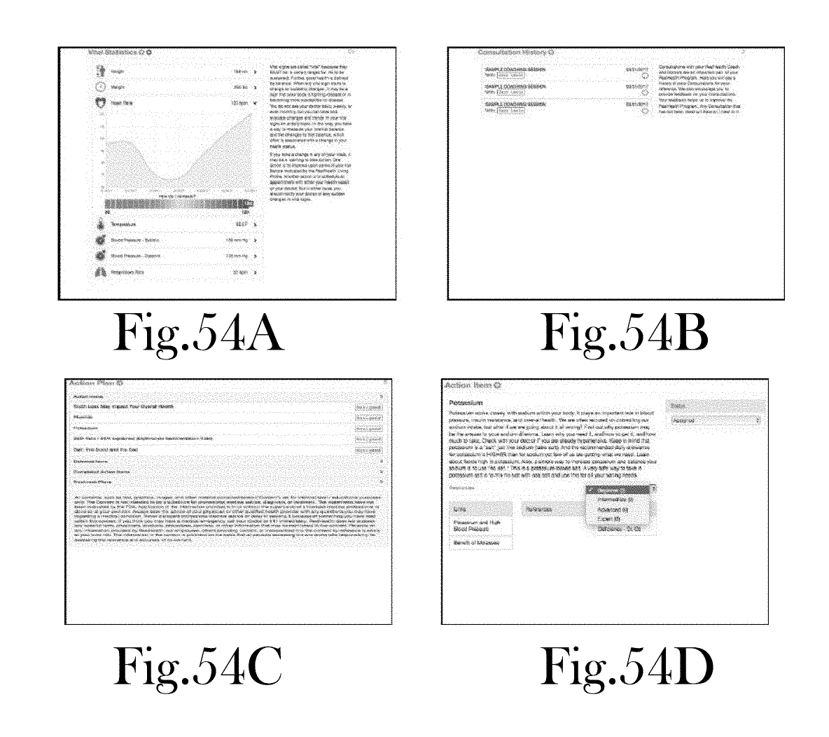

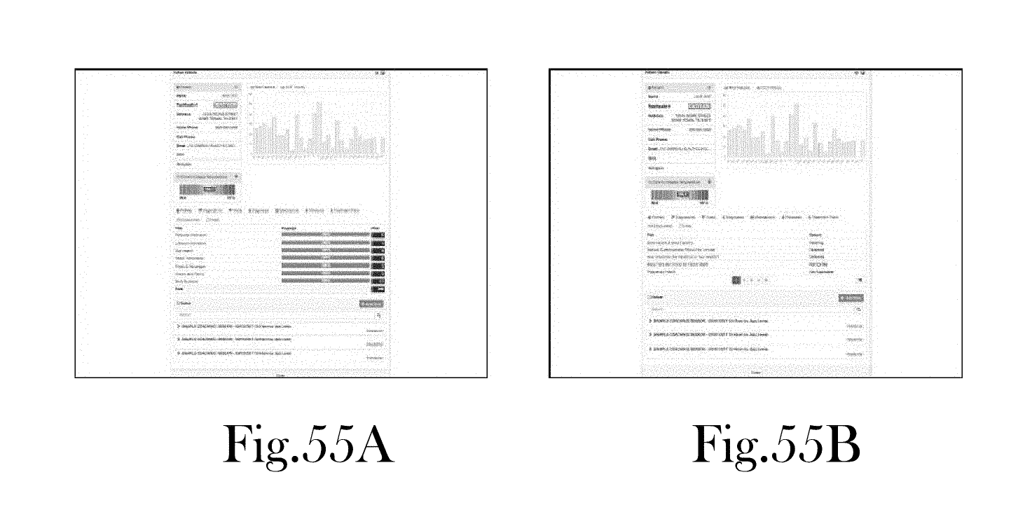

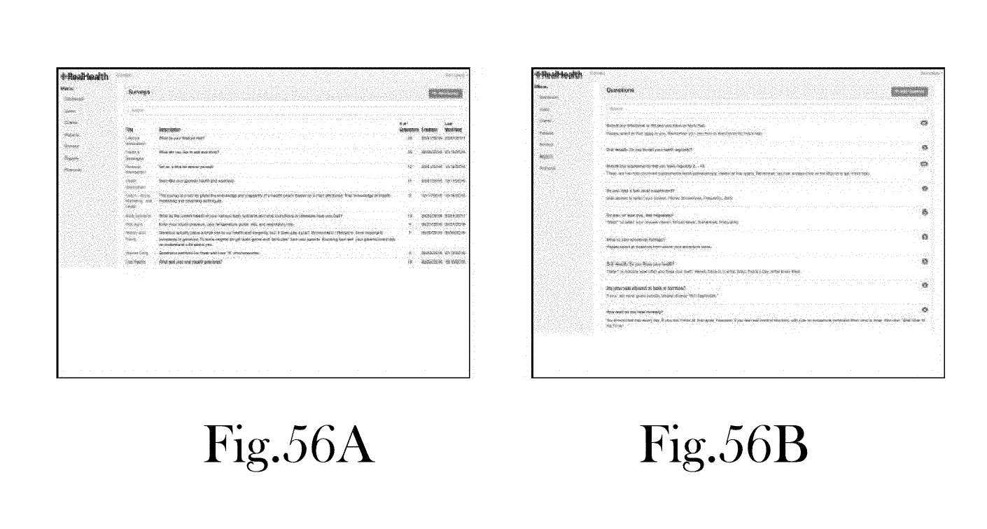

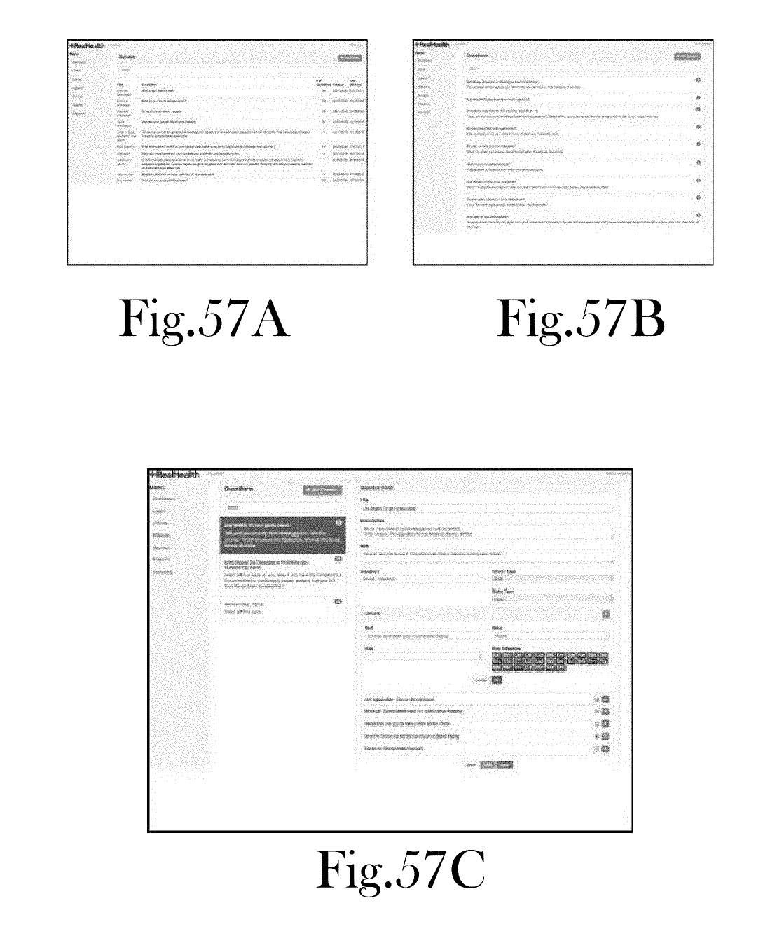

FIGS. 49-57 give examples of the Health Learning Engine Description and Predictive Use. Combination of poor Living Profile™ risk score and high (poor) Chronic Disease Temperature™ guides the provider to perform diagnostic tests for stealth ectopic intracellular pathogens. An example of one such pathogen is chlamydia pneumoniae. A short listing of indications causes or exacerbated by this pathogen is provided in the figures.

In various embodiments, disease specific chronic temperatures are calculated. The methods for calculating a subject's specific disease temperature is the same as for the chronic disease temperature™. A subject's specific chronic disease temperature is obtained by acquiring biomarker values for those markers that are most associated with the specific chronic disease of concern. Accordingly, FIG. 59 is used as a guide for determining which test to perform to determine a specific chronic disease temperature with the marker at the top of the list being the most predictive or most associated specifically to the disease indication based on the prevailing medical literature and the marker at the bottom of the list being the least predictive or least associated specifically to the disease indication based on the prevailing medical literature within that grouping.

Specific Chronic Disease Temperature biomarkers in order of their relevance to the conditions (top row) based on the association between the disease and the marker in the medical research literature. FIG. 59 Key: NRL=neutrophil-to-lymphocyte ratio; B2M=beta-2-microglobulin; Vitamin D=vitamin D (25-hydroxy-); Leptin=Leptin (determine the leptin-to-adiponectin ratio); TNF-α=Tumor necrosis factor alpha; Myelo=Myeloperoxidase; ESR=erythrocyte sedimentation rate; fib=Fibrinogen; neut=neutrophil counts; RDW=Red Blood Cell Distribution Width; plac=LP-PLA2; Hcy=Homocysteine; crp=c-Reactive Protein; uric=Uric Acid; CP=Chlamydophilia Pneumoniae; wbc=White Blood Cell count; fs-Iso=F2-Isoprostanes; L/AR=Leptin-to-Adiponectin Ratio; adipo=Adiponectin (determine the leptin-to-adiponectin ratio); A1C=HbA1C. Heart=all cardiovascular type chronic diseases; Neurodegenerative=all neurodegenerative type chronic diseases, Gastrointestinal=all gastrointestinal type chronic diseases.; Autoimmune=Autoimmune diseases; Inflammation=Chronic diseases of inflammation; Metabolic=Chronic metabolic diseases; Musculoskeletal=Chronic musculoskeletal diseases; Kidney=Chronic kidney disorders; Psychiatric=Chronic mood/neuropsychiatric disorders/diseases; Oral=Chronic oral diseases; Respiratory=Chronic respiratory diseases; Allergy=conditions with an allergic response.

In various exemplary embodiments, the biomarker panel comprises additional biomarkers. Such additional biomarkers may, for example, increase the specificity and/or sensitivity the test. For example, additional biomarkers may be those that are currently evaluated in the clinical laboratory and used in traditional global risk assessment algorithms, such as those from the San Antonio Heart Study, the Framingham Heart Study, the Reynolds Risk Score, the Intermountain Risk Score, and the National Cholesterol Education Program Expert Panel on Detection, Evaluation, and Treatment of High Blood Cholesterol in Adults (Adult Treatment Panel III), also known as NCEP/ATP III. Additional biomarkers suitable for biomarker panels include, without limitation and if not already selected, any combination of biomarkers selected from adiponectin, angiotensin II, complement factor 3, leptin, mRNAx, NFKB, IL-6, MMP-9, eNOS, PPARγ, MCP-1, PAI-I, ICAM/VCAM, E-selectin, P-selectin, von Willebrand factor, sCD40L, proinsulin, glucose, lipids such as free fatty acids, total cholesterol, triglycerides, VLDL, LDL, small dense LDL, oxidized LDL, resistin, HDL, NO, IKB-α, IκB-β, p105, ReIA, MIF, inflammatory cytokines, molecules involved in signaling pathways, and traditional laboratory risk factors. Glucose as used herein includes, without limitation, fasting glucose as well as glucose concentrations taken during and after the oral glucose tolerance test, such as 120 minute Glucose. Insulin as used herein includes, without limitation, fasting insulin and insulin concentrations taken during and after the oral glucose tolerance test, such as 120 minute Insulin.

A biomarker can also be a clinical parameter, although in some embodiments, the biomarker is not included in the definition of “biomarker”. The term “clinical parameter” refers to all non-sample, non-tissue, or non-analyte biomarkers of subject health status or other characteristics, such as, without limitation, age, ethnicity, gender, diastolic blood pressure and systolic blood pressure, family history, height, weight, waist and hip circumference, body-mass index, as well resting heart rate, heart rate variability, microcirculation measurement, homeostatic model assessment (HOMA), HOMA insulin resistance (HOMA-IR), intravenous glucose tolerance (SI(IVGT)), β-cell, macrovascular function, microvascular function, atherogenic index, blood pressure, low-density lipoprotein/high-density lipoprotein ratio, intima-media thickness, and UKPDS risk score.

As described herein, example embodiments of the present general inventive concept can be achieved by a root-cause health creation and optimization methods for subjects with asymptomatic and symptomatic diseases and conditions. The systems and methods can be configured such that the health creation and optimization method repeats as necessary to make the subject optimally healthy.

The systems and methods can be configured to evaluate the risk of accelerated and early health deterioration in a subject using current and past health, phenotype, lifestyle, environmental factors, behavior, family and additional phenotype data inputs pertinent to the subject. For example, data can be gathered in part through a written or electronic health risk assessment (HRA). The HRA provides information on which a subjective health assessment may be made. The HRA can include logic operations that scale and rate the risk of each answer to each question in each survey with a numeric value. A completed HRA (called the Living Profile™) can express the total numeric risk score as a letter grade and provides a letter grade for each part of the Living Profile™.

Example embodiments of the present general inventive concept can be configured to measure the earliest onset of accelerated and early health deterioration in a subject using a panel of biomarkers. Example biomarkers can include physiological, pathophysiological, and pathological markers measureable in serum, exhalant, stool, urine, and tissue.

Although various statistical and/or estimated methods may be used to define a given range of values for carrying out the example methods of the present general inventive concept, and for configuring the structures of the systems to perform the functions described herein, it is possible to define the normal range for a given biomarker as that value or range of values for the biomarker where there is no statistical increase in mortality for a subject with a biomarker of that value or in that range. For example, the normal range for a given biomarker is defined as that value or range of values for the biomarker where there is no statistical increase in morbidity for a subject with a biomarker of that value or in that range. Health risk values can be assigned to a given biomarker for a value or range of values based on available or calculated mortality risk ratios or other available and valid risk assessment measures. A given health risk value for a given biomarker may be referred to as a “temperature increment,” expressed in either Fahrenheit or Celsius units.

Subject aggregate health risk can be assessed by mathematically summing the temperature increments attributable to each biomarker to yield the “total health risk” score. For example, the total sum can be added to 98.6 (for Fahrenheit) or 37 (for Celsius) to yield a subject's Chronic Disease Temperature™ (CDT), according to the formulas and operations of the present general inventive concept (e.g., Biomarker Panels and Calculations of the Chronic/Specific Disease Temperature™).

Statistics and artificial intelligence can be iteratively used to improve the predictive relationship between the HRA risk numerical value (grade) and the biomarker values as expressed by the CDT.

Example embodiments of the present general inventive concept can be configured to interpret the combined risks identified in the Living Profile and the CDT to determine advanced tests to be performed to better identify and treat root-causes of accelerated and early health deterioration. For example, a software system can be configured to gather, store, collate, calculate and interpret health information, and to provide direction for a subject regarding a path of improved health by measuring risks and providing “actions” that help the subject lessen or eliminate a particular risk. The system can be configured to direct a person, either medical or non-medical, to help a subject achieve and identify a path of improved health.

Example embodiments of the present general inventive concept can also be achieved by providing a health software system that enables the subject to rate their experience with the software, the health providers and rate the effectiveness of both the software and providers at creating or improving health and wellbeing.

Example embodiments of the present general inventive concept can also be achieved by providing a health software system that both risk and health stratifies subjects, sub-populations, and entire populations for the purpose of assigned appropriate resources and skill levels to improve and optimize health of the identified population.

Example embodiments of the present general inventive concept can also be achieved by providing a health software system that tracts healthcare spending and savings for a subject, sub-population, and population. The health software system can be configured to display graphical representations of the attributes as illustrated and described herein for sub-populations and populations in addition to information related to a single subject.

The present general inventive concept can be embodied as computer-readable codes on a computer-readable medium. The computer-readable medium can include a computer-readable recording medium and/or a computer-readable transmission medium. The computer-readable recording medium can be any known or later developed data storage device that can store data as a program which can be thereafter read by a computer system. Examples of the computer-readable recording medium include, but are not limited to, read-only memory (ROM), random-access memory (RAM), CD-ROMs, DVDs, jump drives, magnetic tapes, floppy disks, and optical data storage devices. The computer-readable recording medium can be distributed over network coupled computer systems so that the computer-readable code is stored and executed in a distributed fashion. The computer-readable transmission medium can transmit data via wired or wireless data transmission protocols (e.g. applications downloaded or uploaded via the Internet). Also, functional programs, codes, and code segments to accomplish the methods and configurations of the present general inventive concept can be construed and implemented by programmers skilled in the art to which the present general inventive concept pertains without undue experimentation.

It is noted that the simplified diagrams and drawings do not illustrate all the various connections and assemblies of the various components, however, those skilled in the art will understand how to implement such connections and assemblies, based on the illustrated components, figures, and descriptions provided herein. Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Accordingly, while the present general inventive concept has been illustrated by description of several example embodiments, it is not the intention of the applicant to restrict or in any way limit the scope of the inventive concept to such descriptions and illustrations. Instead, the descriptions and drawings herein are to be regarded as illustrative in nature, and not as restrictive, and additional embodiments will readily appear to those skilled in the art upon reading the above description and drawings, and as set forth in the following claims.

What is claimed:

1. A method for determining the chronic or specific disease risk level of a patient, comprising:
acquiring a set of patient blood or related testing and patient health information;
assigning risk values to an acquired set of patient blood or related testing and patient health information based on statistical analysis of morbidity and/or mortality data associated with the acquired set of patient blood or related testing and patient health information;
correlating risk values to a predetermined incremental scale to determine incremental risk value scores for at least one category of health risk;
determining at least one biomarker test to perform and performing the at least one biomarker test on the patient to generate at least one biomarker test results;
determining a raw value for each of the at least one biomarker test results;
comparing the raw value for the at least one biomarker test results to known threshold values related to the biomarker;
determining whether the raw value of the at least one biomarker test results falls within an acceptable range to calculate at least one chronic disease temperature increment for each of the at least one biomarker test results; and
calculating an overall chronic disease temperature value by summing a base chronic disease temperature score with the at least one chronic disease temperature increments.

2. The method according to claim 1, wherein a health risk assessment (HRA) scales and rates the risk value scores and provides a letter grade based on a conventional A-F scale representing a total risk value score.

3. The method according to claim 2, wherein each question from the patient health information is assigned to no more than 100 health categories of risk.

4. The method according to claim 3, wherein each vital sign measurement is assigned to at least one disease or health category known to be associated with that vital sign and each risk value is assigned to the at least one disease or health category known to be associated with the risk value.

5. The method according to claim 4, wherein the at least one biomarker tests include blood borne biomarkers as well as tissue biomarkers.

6. The method according to claim 5, wherein the base chronic disease temperature score is 98.6 degrees F. or 37 degrees C.

7. The method according to claim 6, wherein if the sum of the chronic disease temperature increments is greater than a selected value of degrees, then its value is converted to a value which equals the sum of the at least one chronic disease temperature increments multiplied by the selected value of degrees divided by a maximum chronic disease temperature increment value assigned to each biomarker test.

8. The method according to claim 6, wherein if the sum of the chronic disease temperature increments is less than a selected value of degrees, then the sum of the chronic disease temperature increments may be considered an underestimate of the disease risk level of the patient.

9. The method according to claim 6, wherein the blood borne biomarkers are selected from the group consisting of homocysteine, c-reactive protein, uric acid, myeloperoxidase, beta-w-microglobulin, total white blood cell count, fibrinogen, erythrocyte sedimentation rate, neutrophil count, neutrophil-to-leukocyte ratio, neutrophil-to-lymphocyte ratio, leptin, adiponectin, leptin-to-adiponectin ratio, lp-lpa2, e-GFR, UACR, UAER, microalbuminuria, cystatin C, red blood cell distribution width, 25-hydroxy vitamin D, 1,25-dihydroxyvitamin D, insulin, HbA1C, f2-isoprostanes, TNF-alpha, chlamydophila pneumoniae, other spirochetes, other intracellular infectious species, molds, fungi, species considered benign in certain tissue but pathogenic in others, prions, archaea, obligate species, omega-6 to omega-3 ratio, total cholesterol, N-Terminal pro Brain Natriuretic Peptide, autoantibodies, IgG, IgA, IgM, lipid profiles, triglycerides, Ceruloplasmin, Albumin, Rheumatoid factor (RF), Anti-cyclic citrullinated peptide antibody (CCP), Anti-nuclear antibody (ANA), Complement, NfKBeta, Cryoglobulins, IL-1, IL-6, OxLDL, ADMA/SDMA, Apolipoprotein A-1, Apolipoprotein B, Lipoprotein (a), NMR LipoProfile, sd-LDL, C-Peptide, Fructosamine, TMAO (Trimethylamine N-oxide), Galectin-3, Coenzyme Q10, PSA, Creatine Kinase, toxoplasmosis, other parasites, worms, h-pylori, infectious species associated with lyme disease, nanobacteria, and other infectious species.

10. The method according to claim 9, wherein the tissue biomarkers are selected from the group consisting of nuclear cataract, cortical cataract, subcapsular cataract, glaucoma, macular degeneration, dry eye, amyloidosis, and retinal nerve fiber layer volume and thickness.

11. The method according to claim 1, wherein the acceptable range are those biomarker test results where there is no increase in mortality or morbidity.

12. The method according to claim 1, wherein the acceptable range are those biomarker test results where there is no statistically validated increase in early mortality or morbidity.

13. The method according to claim 1 further comprising:
selecting a disease mitigation treatment plan for the patient based on the results provided from the overall chronic disease temperature value; and
iteratively repeating the method of claim 1 until the overall chronic disease temperature value falls within a predetermined acceptable threshold.

14. The method according to claim 1, further comprising a health learning engine that alters the risk values assigned to the patient health information in response to the calculated chronic disease temperature and individual biomarker values of the chronic disease temperature.

15. The method according to claim 14, where the alteration of the risk values assigned to the patient health information is iteratively altered based on the calculated chronic disease temperature and the individual biomarker values of the chronic disease temperature.

16. The method according to claim 1, further comprising a health learning engine that alters the risk values assigned to the patient blood or related testing in response to the statistical analysis of the morbidity and/or the mortality data.

17. A system for determining the chronic or specific disease risk level of a patient, comprising:
an interface including a display configured to provide a questionnaire related to the patient's health, phenotype, lifestyle, environmental factors, and risk for disease and to gather answers to the questionnaire;
an analyzer that classifies the patient into risk categories and degrees of risk based on the answers to the questionnaire relating to the patient health information and patient blood or related testing to generate overall risk scores for each category of disease, and that matches the risk scores with a set of at least one biomarker tests;
a processor which calculates letter grades for the risk scores and which receives as input raw data related to the set of at least one biomarker tests and generates a set of chronic disease temperature increments as output, and then applies the chronic disease temperature increments to a base chronic disease temperature score to generate an overall chronic disease temperature score;
memory for saving the answers to the questionnaire, the overall risk scores, the results of the biomarker tests, the chronic disease temperature increments and the overall chronic disease temperature score; and
wherein, the system is configured to repeat the steps above after the patient has implemented a disease mitigation program provided by a physician, until the overall chronic disease temperature score falls below a predetermined threshold value.

18. The system according to claim 17, wherein a comparator compares the raw data from the biomarker tests to threshold values for the biomarkers based on known scientific or experimental data.

19. The system according to claim 17, wherein the display includes a graphical representation of the risk value scores which includes a depiction of the assigned letter grade and chronic disease temperature.
